(19) United States
(12) Patent Application Publication
(10) Pub. No.: US 2026/0028586 A1
(43) Pub. Date: Jan. 29, 2026

(54) METHODS FOR PRODUCING CELL POPULATIONS WITH INCREASED NUCLEIC ACID UPTAKE

(71) Applicant: ZEON CORPORATION, Tokyo (JP)

(72) Inventors: Anthony WARD, Richmond, VA (US); Laura HEALEY, Richmond, VA (US); Laurissa OUAGUIA-PALUDAN, Richmond, VA (US); Roberto CAMPOS GONZALEZ, Richmond, VA (US)

(73) Assignee: ZEON CORPORATION, Tokyo (JP)

(21) Appl. No.: 19/351,228

(22) Filed: Oct. 6, 2025

Related U.S. Application Data
(63) Continuation of application No. 17/732,227, filed on Apr. 28, 2022.
Application No. 17/732,227 is a continuation of application No. PCT/US2021/059220, filed on Nov. 12, 2021.
(60) Provisional application No. 63/113,471, filed on Nov. 13, 2020.
(60) Provisional application No. 63/163,585, filed on Mar. 19, 2021.

(51) Int. Cl. C12N 5/0783; A61K 40/11; A61K 40/31; A61K 40/32; A61K 40/42; C12M 1/00

(52) U.S. Cl. CPC C12N 5/0636; A61K 40/11; A61K 40/31; A61K 40/32; A61K 40/4211; A61K 40/4272; C12M 47/04; C12N 2501/2307; C12N 2501/2315; C12N 2510/00

(57) ABSTRACT

A method for obtaining a genetically engineered leukocyte composition is provided. The method includes: (a) enriching a population of large cells from a biological sample obtained from a subject, wherein the biological sample comprises leukocytes without performing density gradient centrifugation; (b) contacting the population of large cells with an activating agent; and (c) transducing the population of large cells with a viral vector comprising a polynucleotide. The enriching comprises an array-based separation comprising a microfluidic device configured for deterministic lateral displacement comprising an array of obstacles, and the obstacles are elongated so that their length perpendicular to bulk fluid flow (P1) is longer than their width parallel to bulk fluid flow (P2) by at least 10%.

### CROSS-REFERENCE

This application is a continuation of U.S. patent application Ser. No. 17/732,227 filed Apr. 28, 2022, which is a continuation of International Application No. PCT/US2021/059220 filed Nov. 12, 2021, which claims priority to U.S. Provisional Patent Application No. 63/113,471 filed Nov. 13, 2020; and U.S. Provisional Patent Application No. 63/163,585 filed Mar. 19, 2021; each of which are incorporated herein by reference in their entirety.

### BACKGROUND

Cell therapies are an important and growing therapeutic option for many patents afflicted with cancer, autoimmune disease, and genetic diseases. These therapies, however, require the ability to produce large amounts of cells transgenic with for nucleic acids that confer a therapeutic benefit.

### SUMMARY

Currently, there exists a need for methods of enriching primary cells from patients and/or individuals such that they possess both high viability and a high ability to be rendered transgenic with exogenous therapeutic nucleic acids. Many cell-based therapies such as chimeric antigen rector T cells (CART-cells) or T cells expressing recombinant T cell receptors are transduced by viruses expressing these recombinant molecules.

Described herein are methods and compositions of cells that have an improved ability to be genetically engineered with exogenous nucleic acids, including nucleic acids with therapeutic potential. The nucleic acids can be delivered by viruses, such as, lentiviruses, adenoviruses, or adeno-associated viruses. Such methods and cell compositions allow for the improved production of therapeutically useful cells allowing for the generation of greater amounts of genetically engineered cells, shorter time frames for the incubation of genetically engineered cells, or both. The methods described herein allow for, in certain embodiments, enrichment based upon size, without the use of toxic density gradient media, and the resulting cell populations show a greater ability to be transfected or transduced with therapeutically relevant genes, that express therapeutically relevant polypeptides.

In one aspect described herein is a method for obtaining a genetically engineered cell composition comprising: (a) providing a biological sample comprising one or more target cells; (b) removing cellular components of a predetermined diameter from the biological sample comprising one or more target cells, wherein the predetermined diameter is 7 micrometers or less, to obtain an enriched target cell population; and (c) contacting the enriched target cell population to an exogenous nucleic acid, thereby providing a genetically engineered target cell population. In some embodiments, the predetermined diameter is 4 micrometers or less. In certain embodiments, the predetermined diameter is about 5 micrometers or less. In certain embodiments, the predetermined diameter is about 4 micrometers or less. In certain embodiments, the biological sample is a fluid comprising one or more cells. In certain embodiments, the one or more cells are human cells. In certain embodiments, the biological sample is selected from the list consisting of a blood related sample, a bone marrow sample, and an adipose sample, and combinations thereof. In certain embodiments, the biological sample is a human biological sample. In certain embodiments, the biological sample is a blood related sample. In certain embodiments, the blood related sample comprises a hematocrit of greater than about 2%. In certain embodiments, the blood related sample comprises a hematocrit of greater than about 4%. In certain embodiments, the blood related sample comprises a hematocrit of less than about 30%. In certain embodiments, the blood related sample is a leukapheresis product. In certain embodiments, the exogenous nucleic acid is a component of a virus. In certain embodiments, the virus is selected from the list consisting of a lentivirus, an adenovirus, or an adeno-associated virus, and combinations thereof. In certain embodiments, the virus is a lentivirus. In certain embodiments, the virus is adenovirus. In certain embodiments, the virus is adeno-associated virus. In certain embodiments, the adeno-associated virus is AAV1, AAV2, AAV3, AAV4, AAV5, AAV6, AAV7, AAV8, or AAV9. In certain embodiments, the virus is a pseudotyped virus. In certain embodiments, the virus comprises a non-viral nucleic acid. In certain embodiments, the non-viral nucleic acid comprises a guide RNA for gene editing. In certain embodiments, the non-viral nucleic acid comprises a sequence encoding a polypeptide component of a gene editing system. Such systems known in the art include TALENs, CRISPR-Cas9, CRISPR-Cas12 and the like. In certain embodiments the non-viral nucleic acid is a CRISPR construct comprising a target strand and a guide strand. In certain embodiments, the non-viral nucleic acid encodes a polypeptide. In certain embodiments, the polypeptide comprises an immunoglobulin, a chimeric antigen receptor, a T cell receptor, a cytokine, or a chemokine. In certain embodiments, the non-viral nucleic acid comprises a chimeric antigen receptor. In certain embodiments, contacting the virus to the enriched target cell population is at a multiplicity of infection 10:1 or greater. In certain embodiments, contacting the virus to the enriched target cell population is at a multiplicity of infection 25:1 or greater. In certain embodiments, contacting the virus to the enriched target cell population is at a multiplicity of infection 50:1 or greater. In certain embodiments, contacting the enriched target cell population to an exogenous nucleic acid occurs by electroporation. In certain embodiments, contacting the enriched target cell population to an exogenous nucleic acid occurs by cell compression. In certain embodiments, the enriched target cell population comprises hematopoietic stem cells. In certain embodiments, the enriched target cell population comprises immune cells. In certain embodiments, the immune cells comprise CD45+ immune cells. In certain embodiments, the immune cells comprise B lymphocytes or T lymphocytes. In certain embodiments, the enriched target cell population comprises CD3+ T lymphocytes. In certain embodiments, the enriched target cell population comprises CD3+, CD4+ T lymphocytes. In certain embodiments, the enriched target cell population comprises CD3+, CD8+ T lymphocytes. In certain embodiments, the enriched target cell population comprises CD3+, CD8+ T lymphocytes. In certain embodiments, the enriched target cell population comprises CD3+, CD8+, CD4+ T lymphocytes. In certain embodiments, the enriched target cell population comprises CD3+, CD8−, CD4− T lymphocytes. In certain embodiments, the enriched target cell population comprises a population of cells comprising at least about 35% CD3+ T lymphocytes. In certain embodiments, the enriched target cell population comprises a population of cells comprising at least about 40% CD3+ T lymphocytes. In certain embodiments, the enriched target cell population comprises natural killer cells. In certain embodiments, the enriched target cell population comprises adipose derived stem cells. In certain embodiments, the enriched target cell population comprises bone marrow derived stem cells. In certain embodiments, the enriched target cell population comprise mesenchymal stem cells. In certain embodiments, the enriched target cell population comprise platelets at a ratio of platelets to target cells of about 500:1 or less. In certain embodiments, the enriched target cell population comprises platelets at a ratio of platelets to target cells of about 100:1 or less. In certain embodiments, the enriched target cell population comprises platelets at a ratio of platelets to target cells of about 10:1 or less. In certain embodiments, the enriched target cell population comprises platelets at a ratio of platelets to target cells of about 5:1 or less. In certain embodiments, the enriched target cell population comprises red blood cells at a ratio of red blood cells to target cells of about 100:1 or more. In certain embodiments, the enriched target cell population comprises red blood cells at a ratio of red blood cells to target cells of about 250:1 or more. In certain embodiments, the enriched target cell population comprises red blood cells at a ratio of red blood cells to target cells of about 500:1 or more. In certain embodiments, the enriched target cell population comprises red blood cells at a ratio of red blood cells to target cells of about 1000:1 or less. In certain embodiments, the method further comprises contacting the enriched target cell population to an activating agent. In certain embodiments, the method comprises contacting the enriched target cell population to an activating agent occurs after removing cellular components of a predetermined diameter or predetermined density from the biological sample comprising one or more target cells, but before contacting the one or more target cells to the exogenous nucleic acid. In certain embodiments, the activating agent comprises one or more of an anti-CD3 antibody, an anti-CD28 antibody, an anti-CD137 antibody, an anti-CD2 antibody, an anti-CD35 antibody, interleukin-2, interleukin-7, or interleukin-15, interleukin-21, interleukin-6, TGFbeta, CD40 ligand, PMA/Ionomycin, Concanavalin A, Pokeweed mitogen, or phytohemagglutinin. In certain embodiments, the activating agent comprises one or more of an anti-CD3 antibody, an anti-CD28 antibody, interleukin-2, interleukin-7, or interleukin-15. In certain embodiments, the genetically engineered target cell exhibits a transduction efficiency greater than density gradient separation methods three-days after contacting the one or more target cells to the exogenous nucleic acid. In certain embodiments, the genetically engineered enriched target cell population exhibits a transduction efficiency of at least about 50% three-days after contacting the one or more target cells to the exogenous nucleic acid. In certain embodiments, the genetically engineered enriched target cell population exhibits a transduction efficiency of at least about 60% three-days after contacting the one or more target cells to the exogenous nucleic acid. In certain embodiments, the genetically engineered target cell exhibits a transduction efficiency greater than density gradient separation methods six-days after contacting the one or more target cells to the exogenous nucleic acid. In certain embodiments, the genetically engineered enriched target cell population exhibits a transduction efficiency of at least about 60% six-days after contacting the one or more target cells to the exogenous nucleic acid. In certain embodiments, the genetically engineered enriched target cell population exhibits a transduction efficiency of at least about 70% six-days after contacting the one or more target cells to the exogenous nucleic acid. In certain embodiments, the method further comprises harvesting the genetically engineered enriched target cell population by day 4 or earlier after contacting the enriched target cell population to an exogenous nucleic acid. In certain embodiments, the method further comprises harvesting the genetically engineered enriched target cell population by day 5 or earlier after contacting the enriched target cell population to an exogenous nucleic acid. In certain embodiments, the method further comprises harvesting the genetically engineered enriched target cell population by day 6 or earlier after contacting the enriched target cell population to an exogenous nucleic acid. In certain embodiments, the method further comprises harvesting the genetically engineered enriched target cell population by day 7 or earlier after contacting the enriched target cell population to an exogenous nucleic acid. In certain embodiments, the method further comprises harvesting the genetically engineered enriched target cell population by day 8 or earlier after contacting the enriched target cell population to an exogenous nucleic acid. In certain embodiments, the cells are harvested from day 3 to day 8 after contacting the enriched target cell population to an exogenous nucleic acid. In certain embodiments, the cells are harvested from day 3 to day 7 after contacting the enriched target cell population to an exogenous nucleic acid. In certain embodiments, the cells are harvested from day 3 to day 6 after contacting the enriched target cell population to an exogenous nucleic acid. In certain embodiments, the cells are harvested from day 3 to day 5 after contacting the enriched target cell population to an exogenous nucleic acid. In certain embodiments, at least 1×10⁸ genetically engineered target cells are harvested. In certain embodiments, at least 1×10⁷ genetically engineered target cells are harvested. In certain embodiments, removing the cellular components of a predetermined diameter from the biological sample does not employ use of a density gradient medium. In certain embodiments, removing the cellular components of a predetermined diameter from the biological sample employs deterministic lateral displacement.

In another aspect described herein is a method for obtaining a genetically engineered cell composition comprising: (a) providing a biological sample comprising one or more target cells; (b) removing cellular components of a predetermined diameter from the biological sample comprising one or more target cells, wherein the predetermined density is 1.1 grams per milliliter or less; to obtain an enriched target cell population; and (c) contacting the enriched target cell population to an exogenous nucleic acid, thereby providing genetically engineered enriched target cells. In certain embodiments, the predetermined density is about 1.09 grams per milliliter or less. In certain embodiments, the predetermined density is about 1.08 grams per milliliter or less. In certain embodiments, the biological sample is a fluid comprising one or more cells. In certain embodiments, the one or more cells are human cells. In certain embodiments, the biological sample is selected from the list consisting of a blood related sample, a bone marrow sample, and an adipose sample, and combinations thereof. In certain embodiments, the biological sample is a human biological sample. In certain embodiments, the biological sample is a blood related sample. In certain embodiments, the blood related sample comprises a hematocrit of greater than about 2%. In certain embodiments, the blood related sample comprises a hematocrit of greater than about 4%. In certain embodiments, the blood related sample comprises a hematocrit of less than about 30%. In certain embodiments, the blood related sample is a leukapheresis product. In certain embodiments, the exogenous nucleic acid is a component of a virus. In certain embodiments, the virus is selected from the list consisting of a lentivirus, an adenovirus, or an adeno-associated virus, and combinations thereof. In certain embodiments, the virus is a lentivirus. In certain embodiments, the virus is adenovirus. In certain embodiments, the virus is adeno-associated virus. In certain embodiments, the virus comprises a non-viral nucleic acid. In certain embodiments, the non-viral nucleic acid is a CRISPR construct comprising a target strand and a guide strand. In certain embodiments, the non-viral nucleic acid encodes a polypeptide. In certain embodiments, the polypeptide comprises an immunoglobulin, a chimeric antigen receptor, a T cell receptor, a cytokine, or a chemokine. In certain embodiments, the non-viral nucleic acid comprises a chimeric antigen receptor. In certain embodiments, contacting the virus to the enriched target cell population is at a multiplicity of infection 10:1 or greater. In certain embodiments, contacting the virus to the enriched target cell population is at a multiplicity of infection 25:1 or greater. In certain embodiments, contacting the virus to the enriched target cell population is at a multiplicity of infection 50:1 or greater. In certain embodiments, contacting the enriched target cell population to an exogenous nucleic acid occurs by electroporation. In certain embodiments, contacting the enriched target cell population to an exogenous nucleic acid occurs by cell compression. In certain embodiments, the enriched target cell population comprises hematopoietic stem cells. In certain embodiments, the enriched target cell population comprises immune cells. In certain embodiments, the immune cells comprise CD45+ immune cells. In certain embodiments, the immune cells comprise B lymphocytes or T lymphocytes. In certain embodiments, the enriched target cell population comprises CD3+ T lymphocytes. In certain embodiments, the enriched target cell population comprises CD3+, CD4+ T lymphocytes. In certain embodiments, the enriched target cell population comprises CD3+, CD8+ T lymphocytes. In certain embodiments, the enriched target cell population comprises CD3+, CD8+ T lymphocytes. In certain embodiments, the enriched target cell population comprises CD3+, CD8+, CD4+ T lymphocytes. In certain embodiments, the enriched target cell population comprises CD3+, CD8−, CD4− T lymphocytes. In certain embodiments, the enriched target cell population comprises a population of cells comprising at least about 35% CD3+ T lymphocytes. In certain embodiments, the enriched target cell population comprises a population of cells comprising at least about 40% CD3+ T lymphocytes. In certain embodiments, the enriched target cell population comprises natural killer cells. In certain embodiments, the enriched target cell population comprises adipose derived stem cells. In certain embodiments, the enriched target cell population comprises bone marrow derived stem cells. In certain embodiments, the enriched target cell population comprise mesenchymal stem cells. In certain embodiments, the enriched target cell population comprise platelets at a ratio of platelets to target cells of about 500:1 or less. In certain embodiments, the enriched target cell population comprises platelets at a ratio of platelets to target cells of about 100:1 or less. In certain embodiments, the enriched target cell population comprises platelets at a ratio of platelets to target cells of about 10:1 or less. In certain embodiments, the enriched target cell population comprises platelets at a ratio of platelets to target cells of about 5:1 or less. In certain embodiments, the enriched target cell population comprises red blood cells at a ratio of red blood cells to target cells of about 100:1 or more. In certain embodiments, the enriched target cell population comprises red blood cells at a ratio of red blood cells to target cells of about 250:1 or more. In certain embodiments, the enriched target cell population comprises red blood cells at a ratio of red blood cells to target cells of about 500:1 or more. In certain embodiments, the enriched target cell population comprises red blood cells at a ratio of red blood cells to target cells of about 1000:1 or less. In certain embodiments, the method further comprises contacting the enriched target cell population to an activating agent. In certain embodiments, the method comprises contacting the enriched target cell population to an activating agent occurs after removing cellular components of a predetermined diameter or predetermined density from the biological sample comprising one or more target cells, but before contacting the one or more target cells to the exogenous nucleic acid. In certain embodiments, the activating agent comprises one or more of an anti-CD3 antibody, an anti-CD28 antibody, an anti-CD137 antibody, an anti-CD2 antibody, an anti-CD35 antibody, interleukin-2, interleukin-7, or interleukin-15, interleukin-21, interleukin-6, TGFbeta, CD40 ligand, PMA/Ionomycin, Concanavalin A, Pokeweed mitogen, or phytohemagglutinin. In certain embodiments, the activating agent comprises one or more of an anti-CD3 antibody, an anti-CD28 antibody, interleukin-2, interleukin-7, or interleukin-15. In certain embodiments, the genetically engineered target cell exhibits a transduction efficiency greater than density gradient separation methods three-days after contacting the one or more target cells to the exogenous nucleic acid. In certain embodiments, the genetically engineered enriched target cell population exhibits a transduction efficiency of at least about 50% three-days after contacting the one or more target cells to the exogenous nucleic acid. In certain embodiments, the genetically engineered enriched target cell population exhibits a transduction efficiency of at least about 60% three-days after contacting the one or more target cells to the exogenous nucleic acid. In certain embodiments, the genetically engineered target cell exhibits a transduction efficiency greater than density gradient separation methods six-days after contacting the one or more target cells to the exogenous nucleic acid. In certain embodiments, the genetically engineered enriched target cell population exhibits a transduction efficiency of at least about 60% six-days after contacting the one or more target cells to the exogenous nucleic acid. In certain embodiments, the genetically engineered enriched target cell population exhibits a transduction efficiency of at least about 70% six-days after contacting the one or more target cells to the exogenous nucleic acid. In certain embodiments, the method further comprises harvesting the genetically engineered enriched target cell population by day 4 or earlier after contacting the enriched target cell population to an exogenous nucleic acid. In certain embodiments, the method further comprises harvesting the genetically engineered enriched target cell population by day 5 or earlier after contacting the enriched target cell population to an exogenous nucleic acid. In certain embodiments, the method further comprises harvesting the genetically engineered enriched target cell population by day 6 or earlier after contacting the enriched target cell population to an exogenous nucleic acid. In certain embodiments, the method further comprises harvesting the genetically engineered enriched target cell population by day 7 or earlier after contacting the enriched target cell population to an exogenous nucleic acid. In certain embodiments, the method further comprises harvesting the genetically engineered enriched target cell population by day 8 or earlier after contacting the enriched target cell population to an exogenous nucleic acid. In certain embodiments, the cells are harvested from day 3 to day 8 after contacting the enriched target cell population to an exogenous nucleic acid. In certain embodiments, the cells are harvested from day 3 to day 7 after contacting the enriched target cell population to an exogenous nucleic acid. In certain embodiments, the cells are harvested from day 3 to day 6 after contacting the enriched target cell population to an exogenous nucleic acid. In certain embodiments, the cells are harvested from day 3 to day 5 after contacting the enriched target cell population to an exogenous nucleic acid. In certain embodiments, at least 1×10⁸ genetically engineered target cells are harvested. In certain embodiments, at least 1×10⁷ genetically engineered target cells are harvested. In certain embodiments, removing the cellular components of a predetermined diameter from the biological sample does not employ use of a density gradient medium. In certain embodiments, removing the cellular components of a predetermined diameter from the biological sample employs deterministic lateral displacement.

Also described herein is a cell population comprising one or more enriched target cells, platelet cells and red blood cells, wherein the ratio of platelets to enriched target cells is less than about 500:1 and the ratio of red blood cells to enriched target cells is greater than about 50:1, wherein greater than about 60% of the enriched target cells comprise an exogenous nucleic acid. In certain embodiments, the exogenous nucleic acid encodes a polypeptide. In certain embodiments, the cell population expresses the polypeptide. In certain embodiments, the exogenous polypeptide comprises an immunoglobulin, a chimeric antigen receptor, a T cell receptor, a cytokine, or a chemokine. In certain embodiments, the exogenous polypeptide comprises a chimeric antigen receptor. In certain embodiments, the enriched target cells comprise human cells. In certain embodiments, the enriched target cells, platelet cells, and red blood cells comprise human cells. In certain embodiments, the ratio of platelets to enriched target cells is less than about 100:1. In certain embodiments, the ratio of platelets to enriched target cells is less than about 10:1. In certain embodiments, the ratio of platelets to enriched target cells is less than about 5:1. In certain embodiments, the ratio of red blood cells to enriched target cells is greater than about 100:1. In certain embodiments, the ratio of red blood cells to enriched target cells is greater than about 250:1. In certain embodiments, the ratio of red blood cells to enriched target cells is greater than about 500:1. In certain embodiments, the ratio of red blood cells to enriched target cells is greater than about 1,000:1. In certain embodiments, the one or more enriched target cells comprise immune cells. In certain embodiments, the immune cells comprise CD45+ immune cells. In certain embodiments, the CD45+ immune cells at least about 35% CD3+ T lymphocytes. In certain embodiments, the CD45+ immune cells at least about 40% CD3+ T lymphocytes. In certain embodiments, the immune cells comprise B lymphocytes or T lymphocytes. In certain embodiments, the enriched target cell population comprises CD3+ T lymphocytes. In certain embodiments, the one or more target cells possess the capacity to divide at least 3 time before exhaustion. In certain embodiments, the cell population further comprises Interleukin 7. In certain embodiments, the interleukin-7 is present at a concentration of at least 25 ng per mL. In certain embodiments, the cell population further comprises Interleukin-15. In certain embodiments, the interleukin-15 is present at a concentration of at least 25 ng per mL.

In another aspect described herein, is a cell population isolated from a sample of a subject, the cell population comprising cells comprising a heterologous DNA, wherein compared to a buffy coat cell population isolated from the sample by density gradient centrifugation: (a) a number of white blood cells in the cell population is at least 2 times more than a number of white blood cells in the buffy coat cell population; (b) a number of T cells in the cell population is at least 2 times more than a number of T cells in the buffy coat cell population; (c) a ratio of red blood cells to T cells in the cell population is at least 5 times less than a ratio of red blood cells to T cells in the buffy coat cell population; (d) a ratio of platelets to T cells in the cell population is at least 5 times less than a ratio of platelets to T cells in the buffy coat cell population; (e) a percentage of senescent cells in the cell population is at least 10% less than a percentage of senescent cells in the buffy coat cell population; (f) a percentage of exhausted cells in the cell population is at least 10% less than a percentage of exhausted cells in the buffy coat cell population; (g) a percentage of T effector memory cells that express CD45Ra in the cell population is at least 10% less than a percentage of T effector memory cells that express CD45Ra in the buffy coat cell population; (h) a percentage of T central memory cells in the cell population is at least 10% higher than a percentage of T central memory cells in the buffy coat cell population; (i) a percentage of cells in the cell population that are T central memory cells or T effector memory cells is at least 10% higher than a percentage of cells in the buffy coat cell population that are T central memory cells or T effector memory cells; (j) a percentage of cells comprising the heterologous DNA in the cell population is at least 20% higher than a percentage of cells comprising the heterologous DNA in the buffy coat cell population; and the cell population and the buffy coat cell population are transduced with a viral vector comprising the heterologous DNA; (k) the cell population is capable of expanding to comprise at least 2×10e9 T cells comprising the heterologous DNA in at least 30% less time than the buffy coat cell population; and the cell population and the buffy coat cell population are transduced with a viral vector comprising the heterologous DNA; (1) the cell population expresses more interferon gamma than the buffy coat cell population; (m) the cell population expresses more GM-CSF than the buffy coat cell population; (n) the cell population secretes less IL-6 than the buffy coat cell population; (o) the cell population secretes less MCP-1 than the buffy coat cell population; (p) the cell population secretes less IL-1Ra than the buffy coat cell population; (q) the cell population comprises a higher mean absolute telomer length than the buffy coat cell population; or (r) the cell population comprises T cells comprising a higher mean absolute telomer length than T cells purified from the buffy coat cell population.

In certain embodiments, a number of white blood cells in the cell population is at least 2 times more than a number of white blood cells in the buffy coat cell population. In certain embodiments, a number of T cells in the cell population is at least 2 times more than a number of T cells in the buffy coat cell population. In certain embodiments, a ratio of red blood cells to T cells in the cell population is at least 5 times less than a ratio of red blood cells to T cells in the buffy coat cell population. In certain embodiments, a ratio of platelets to T cells in the cell population is at least 5 times less than a ratio of platelets to T cells in the buffy coat cell population. In certain embodiments, a percentage of senescent cells in the cell population is at least 10% less than a percentage of senescent cells in the buffy coat cell population. In certain embodiments, a percentage of exhausted cells in the cell population is at least 10% less than a percentage of exhausted cells in the buffy coat cell population. In certain embodiments, a percentage of T effector memory cells that express CD45Ra in the cell population is at least 10% less than a percentage of T effector memory cells that express CD45Ra in the buffy coat cell population. In certain embodiments, a percentage of T central memory cells in the cell population is at least 10% higher than a percentage of T central memory cells in the buffy coat cell population. In certain embodiments, a percentage of cells in the cell population that are T central memory cells or T effector memory cells is at least 10% higher than a percentage of cells in the buffy coat cell population that are T central memory cells or T effector memory cells.

In certain embodiments, a percentage of cells comprising the heterologous DNA in the cell population is at least 20% higher than a percentage of cells comprising the heterologous DNA in the buffy coat cell population; and the cell population and the buffy coat cell population are transduced with a viral vector comprising the heterologous DNA. In certain embodiments, the cell population is capable of expanding to comprise at least 2×10⁹ T cells comprising the heterologous DNA in at least 30% less time than the buffy coat cell population; and the cell population and the buffy coat cell population are transduced with a viral vector comprising the heterologous DNA. In certain embodiments, the cell population expresses more interferon gamma than the buffy coat cell population. In certain embodiments, the cell population expresses more GM-CSF than the buffy coat cell population. In certain embodiments, the cell population secretes less IL-6 than the buffy coat cell population. In certain embodiments, the cell population secretes less MCP-1 than the buffy coat cell population. In certain embodiments, the cell population secretes less IL-1Ra than the buffy coat cell population. In certain embodiments, the cell population comprises a higher mean absolute telomer length than the buffy coat cell population. In certain embodiments, the cell population comprises T cells comprising a higher mean absolute telomer length than T cells purified from the buffy coat cell population.

In certain embodiments, the heterologous DNA comprises an inverted terminal repeat sequence or a long terminal repeat sequence. In certain embodiments, the density gradient centrifugation comprises layering the sample over an aqueous solution comprising sodium diatrizoate, disodium calcium EDTA, and a neutral, highly branched, high-mass, hydrophilic polysaccharide having a density of about 1.078 g/ml [e.g. Ficoll]. In certain embodiments, the sample is a leukopak. In certain embodiments, the sample is residual leukocytes from a platelet donation. In certain embodiments, the sample is a blood sample. In certain embodiments, the hematocrit of the blood sample is >2%. In certain embodiments, the hematocrit of the blood sample is >4%. In certain embodiments, the hematocrit of the blood sample is <30%. In certain embodiments, the sample is a leukophoresis or apheresis sample. In certain embodiments, the sample is an adipose sample or a bone marrow sample.

In certain embodiments, the subject is a human. In certain embodiments, the subject is a healthy individual. In certain embodiments, the subject has a cancer. In certain embodiments, the cancer is a leukemia. In certain embodiments, the viral vector is a lentiviral vector. In certain embodiments, the viral vector is an adenovirus vector. In certain embodiments, the viral vector is an adeno-associated virus vector. In certain embodiments, the heterologous DNA encodes a CRISPR guide RNA. In certain embodiments, the heterologous DNA encodes an siRNA or a miRNA. In certain embodiments, the heterologous DNA encodes a polypeptide. In certain embodiments, the polypeptide is a chimeric antigen receptor. In certain embodiments, the chimeric antigen receptor is selected from the list consisting of tisagenlecleucel, axicabtagene ciloleucel, brexucabtagene autoleucel, lisocabtagene maraleucel, idecabtagene vicleucel, and combinations thereof. In certain embodiments, the polypeptide is an immunoglobulin, a T cell receptor, a cytokine, or a chemokine. In certain embodiments, at least 90% of the cells of the cell population are viable.

In another aspect described herein, is a method for obtaining a genetically engineered leukocyte composition comprising: (a) enriching a population of large cells from a biological sample comprising leukocytes without performing density gradient centrifugation; (b) contacting the population of large cells with an activating agent; and (c) transducing the population of large cells with a viral vector comprising a polynucleotide. In certain embodiments, the large cells have a diameter of at least 4 μm. In certain embodiments, the large cells have a diameter of at least 5 μm. In certain embodiments, the large cells have a diameter of at least 7 μm.

In another aspect described herein, is a method for obtaining a genetically engineered leukocyte composition comprising: (a) removing components below a predetermined size from a biological sample from a subject comprising leukocytes without performing density gradient centrifugation to generate a population of large cells; (b) contacting the population of large cells with an activating agent; and (c) transducing the population of large cells with a viral vector comprising a polynucleotide. In certain embodiments, the predetermined size is 4 μm. In certain embodiments, the predetermined size is 5 μm. In certain embodiments, the predetermined size is 7 μm. In certain embodiments, the biological sample comprises human cells. In certain embodiments, the biological sample is a leukopak. In certain embodiments, wherein the biological sample is residual leukocytes from a platelet donation. In certain embodiments, the biological sample is a blood sample. In certain embodiments, the blood sample has a hematocrit of >2%. In certain embodiments, the blood sample has a hematocrit of >4%. In certain embodiments, the blood sample has a hematocrit of <30%. In certain embodiments, wherein the biological sample is a leukophoresis or apheresis sample. In certain embodiments, the biological sample is an adipose sample or a bone marrow sample. In certain embodiments, the subject is a human. In certain embodiments, the subject is a healthy individual. In certain embodiments, the subject has a cancer. In certain embodiments, the cancer is a leukemia.

In certain embodiments, the viral vector is a lentiviral vector. In certain embodiments, the viral vector is a adenovirus vector. In certain embodiments, the viral vector is a adeno-associated virus vector. In certain embodiments, the polynucleotide is a heterologous DNA or a heterologous RNA. In certain embodiments, the polynucleotide encodes a CRISPR guide RNA. In certain embodiments, the polynucleotide encodes an siRNA or a miRNA. In certain embodiments, the polynucleotide encodes a polypeptide. In certain embodiments, the polypeptide is a chimeric antigen receptor. In certain embodiments, the chimeric antigen receptor is selected from the list consisting of tisagenlecleucel, axicabtagene ciloleucel, brexucabtagene autoleucel, lisocabtagene maraleucel, idecabtagene vicleucel, and combinations thereof. In certain embodiments, the polypeptide is an immunoglobulin, a T cell receptor, a cytokine, or a chemokine. In certain embodiments, at least 90% of the cells of the genetically engineered leukocyte composition are viable.

In certain embodiments, the enriching comprises an array-based separation, an acoustophoretic isolation, or an affinity separation. In certain embodiments, the array-based separation comprises a microfluidic device configured for deterministic lateral displacement. In certain embodiments, the microfluidic device comprises a plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle. In certain embodiments, the device comprises at least 50 arrays of obstacles. In certain embodiments, the device comprises at least 50 arrays of obstacles arranged in parallel. In certain embodiments, the plurality of obstacles comprises at least 50 rows of obstacles. In certain embodiments, the plurality of obstacles comprises at least 50 columns of obstacles.

In certain embodiments, the microfluidic device comprises an array of posts having a diameter of about 20 μm. In certain embodiments, a buffer flows continuously through the microfluidic device. In certain embodiments, the microfluidic device operates in oscillatory flow conditions. In certain embodiments, the flow rate through the microfluidic device is at least about 500 mL per hour. In certain embodiments, the flow rate through the microfluidic device is at least about 1000 mL per hour. In certain embodiments, the microfluidic device comprises an array of asymmetric hexagonal obstacles. In certain embodiments, the microfluidic device comprises a plurality of obstacles having a diamond shape. In certain embodiments, the microfluidic device comprises a plurality of obstacles having a circular or ellipsoid shape. In certain embodiments, each obstacle of the plurality of obstacles has a diamond, circular, ellipsoid, or hexagonal shape. In certain embodiments, each obstacle of plurality of obstacles has a horizontal P1 length approximately parallel to the direction of fluid flow that is longer than a P2 length approximately perpendicular to the direction of fluid flow. In certain embodiments, each obstacle of the plurality of obstacles has an elongated hexagonal shape.

In certain embodiments, P1 is about 10 μm to about 60 μm and P2 is about 10 μm to about 30 μm. In certain embodiments, P1 is about 40 μm and P2 is about 20 μm. In certain embodiments, P1 is 50% to 150% longer than P2. In certain embodiments, the obstacles in a column are separated by a G1 gap of about 22 μm and the obstacles in a row of obstacles are separated by a G2 gap of about 17 μm. In certain embodiments, the microfluidic device comprises a plurality of obstacles having vertices that extend into parallel gaps such that the gaps are flanked on either side by one or more vertices pointing toward one another but not directly opposite one another. In certain embodiments, the microfluidic device comprises a plurality of obstacles have vertices that extend into perpendicular gaps such that the gaps are flanked on either side by vertices pointing toward one another and that are directly opposite one another. In certain embodiments, the microfluidic device comprises a plurality of obstacles arranged such that the tilt angle is 1/100, indicating that the obstacles are perfectly aligned in every 100th row. In certain embodiments, the microfluidic device comprises a plurality of obstacles arranged into at least 50 columns. In certain embodiments, the microfluidic device comprises a plurality of obstacles arranged into at least about 50 rows. In certain embodiments, the microfluidic device comprises a first and/or second planar support which comprise at least 20 embedded channels.

In certain embodiments, the activating agent comprises one or more of an anti-CD3 antibody, an anti-CD28 antibody, interleukin-2, interleukin-7, or interleukin-15. In certain embodiments, the anti-CD3 antibody or the anti-CD28 antibody are conjugated to a solid support. In certain embodiments, the solid support is a magnetic bead. In certain embodiments, the contacting the population of large cells with the anti-CD3 antibody or the anti-CD28 antibody conjugated to a solid support further comprises affinity enrichment of leukocytes expressing CD3 or CD28. In certain embodiments, the transducing comprises contacting the population of large cells with the viral vector comprising a polynucleotide at a multiplicity of infection of at least 5. In certain embodiments, the method further comprises treating the biological sample with a nuclease prior to (a). In certain embodiments, the method further comprises freezing the population of large cells and thawing the population of large cells. In certain embodiments, the method further comprises: (a) culturing the population of large cells. In certain embodiments, the culturing is for at least 2, 3, 4, 5, 6, 7, 8, 9, or 10 days. In certain embodiments, the culturing is for no more than 15, 10, 9, 8, 7, 6, 5, 4 or 3 days.

In certain embodiments, at least 70% of the T-cells express the polynucleotide/polypeptide. In certain embodiments, the percentage of cells expressing the polypeptide is determined by flow cytometry. In certain embodiments, the genetically engineered leukocyte composition comprises at least 1×10⁹ T cells. In certain embodiments, at least 75% of the T cells of the genetically engineered leukocyte composition are T central memory cells or T effector memory cells after 6 days of culturing. In certain embodiments, at least 85% of the T cells of the genetically engineered leukocyte composition are T central memory cells or T effector memory cells after 9 days of culturing. In certain embodiments, the method further comprises freezing the genetically engineered leukocyte population and thawing the genetically engineered leukocyte population. In certain embodiments, the method further comprises administering the genetically engineered leukocyte population to an individual afflicted with a tumor or cancer.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A illustrates recovery of peripheral blood mononuclear cells by Deterministic Lateral Displacement (DLD) or density gradient centrifugation (Ficoll).

FIG. 1B illustrates % of CD3 cells amongst total CD45+ cells after enrichment by DLD or density gradient centrifugation (Ficoll).

FIG. 1C illustrates higher viral transduction after enrichment by DLD or density gradient centrifugation (Ficoll) at Day 3 and Day 6.

FIG. 2 shows fluorescence microscopy of GFP positive cells at day 3 after transduction by virus for cells enriched by DLD or density gradient centrifugation

FIG. 3 illustrates increased total numbers of virally transduced T cells 3 and 6 days post viral transduction for DLD compared to density gradient centrifugation.

FIG. 4 illustrates improved recovery of all white blood cells (WBC)/CD 45+ cells from leukopaks enriched according to the systems and methods herein compared to Ficoll.

FIG. 5 illustrates that systems and methods herein recover more of the CD4 less differentiated naïve and central memory subsets from leukopaks, at the start of the cell therapy manufacturing at day 0, versus Ficoll.

FIG. 6 illustrates improved recovery of all white blood cells (WBC)/CD 45+ cells and CD 3+ T cells from lower WBC count patient leukopaks enriched according to the systems and methods herein compared to Ficoll.

FIG. 7 illustrates that cells enriched according to the systems and methods herein from cancer patient leukopaks have fewer platelets and red blood cells compared to Ficoll.

FIG. 8 illustrates that cells enriched according to the systems and methods herein integrate lentivirus more readily compared to Ficoll preparations.

FIG. 9 illustrates that cells enriched according to the systems and methods herein integrate lentivirus more readily and express reporter genes at an earlier timepoint compared to Ficoll preparations.

FIG. 10 illustrates that cells enriched according to the systems and methods herein are more receptive to viral transduction compared to Ficoll preparations.

FIG. 11 illustrates that cells enriched according to the systems and methods herein, lentiviral transduced, and expanded, produce more dose equivalents of therapeutic leukocytes at an earlier time point compared to Ficoll preparations.

FIG. 12 illustrates that cell populations enriched according to the systems and methods herein, and treated with high integration lentivirus, have fewer terminally differentiated cells compared to Ficoll preparations.

FIG. 13 illustrates that systems and methods herein recover more of the less differentiated naïve and central memory subsets from normal donor leukopaks and result in fewer terminally differentiated cells compared to Ficoll preparations.

FIG. 14 illustrates that viable CD 3+ memory cell populations enriched according to the systems and methods herein, and treated with high integration lentivirus, retain relative populations of T memory cells compared to Ficoll preparations.

FIG. 15 illustrates that cell populations enriched according to the systems and methods herein show two-fold reduced senescence and exhaustion with significantly less PD1/Tim3 co-expression at day 13 of culture, and fewer cells entering TEMRA state (T effector memory cells that express CD45Ra) compared to Ficoll preparations.

FIG. 16 illustrates that cell populations enriched according to the systems and methods herein have normal or increased killing capacity compared to Ficoll preparations.

FIG. 17 illustrates that cell populations enriched according to the systems and methods herein have a more favorable cytokine expression during expansion and thus a more favorable safety profile compared to Ficoll preparations.

FIG. 18 illustrates that cell populations enriched according to the systems and methods herein have a more favorable cytokine expression during expansion and thus a more favorable safety profile compared to Ficoll preparations, as demonstrated with cytokine release with CD19 CAR-T constructs (+/− functional CD28 signaling domain).

FIG. 19 illustrates that cell populations enriched according to the systems and methods herein have a more favorable cytokine expression during expansion and thus a more favorable safety profile compared to Ficoll preparations, as demonstrated with cytokine release with TCR-T constructs and lentiviral-GFP controls.

FIG. 20 summarizes various advantages of cell populations enriched according to the systems and methods herein compared to Ficoll preparations.

FIG. 21A and FIG. 21B illustrate that cells enriched according to the systems and methods herein possess longer Telomere length compared to Ficoll, indicating greater expansion capability. FIG. 21C illustrates that T cells enriched according to the systems and methods herein possess longer Telomere length compared to Ficoll. Assays were conducted using qPCR analysis for absolute Telomere Length (aTL).

FIG. 22 illustrates an embodiment of a DLD separation device that may be used for enriching cell populations.

FIG. 23 illustrates symmetric and asymmetric obstacle placements for example obstacle shapes.

FIG. 24 illustrates enhanced viral transduction efficiency of frozen and thawed T-cells isolated by DLD compared with T-cells isolated by Ficoll.

### DETAILED DESCRIPTION

In one aspect described herein is a method for obtaining a genetically engineered cell composition comprising: (a) providing a biological sample comprising one or more target cells; (b) removing cellular components of a predetermined diameter from the biological sample comprising one or more target cells, wherein the predetermined diameter is 7 micrometers or less, to obtain an enriched target cell population; and (c) contacting the enriched target cell population to an exogenous nucleic acid, thereby providing a genetically engineered target cell population. In some embodiments, the predetermined size is 4 micrometers or less.

In another aspect described herein is a method for obtaining a genetically engineered cell composition comprising: (a) providing a biological sample comprising one or more target cells; (b) removing cellular components of a predetermined diameter from the biological sample comprising one or more target cells, wherein the predetermined density is 1.1 grams per milliliter or less; to obtain an enriched target cell population; and (c) contacting the enriched target cell population to an exogenous nucleic acid, thereby providing genetically engineered enriched target cells.

Also described herein is a cell population comprising one or more enriched target cells, platelet cells and red blood cells, wherein the ratio of platelets to enriched target cells is less than about 500:1 and the ratio of red blood cells to enriched target cells is greater than about 50:1, wherein greater than about 50%, 55%, 60%, 65%, 70%, or 75% of the enriched target cells comprise an exogenous nucleic acid.

### Certain Terms

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and advantageous characteristic(s) of the claimed invention. Compositions for treating or preventing a given disease can consist essentially of the recited active ingredient, exclude additional active ingredients, but include other non-active components such as excipients, carriers, or diluents. “Consisting of” shall mean excluding more than trace elements of other ingredients and substantial method steps. Embodiments defined by each of these transition terms are within the scope of this disclosure.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The term “target cells” refers to a type of cell, cell population, or composition of cells which are the desired cells to be collected, enriched, isolated, or separated by the present invention. Target cells represent cells that various procedures described herein require or are designed to purify, collect, engineer etc. What the specific cells are will depend on the context in which the term is used. For example, if the objective of a procedure is to isolate a particular kind of stem cell, that cell would be the target cell of the procedure. The terms “target cells” and “desired cells” are interchangeable and have the same meaning regarding the present invention. Target cells can exist in a genus-species relationship. For example, if target cells comprised leukocytes, the target cells would include T cells.

The term “antibody” or “immunoglobulin” herein is used in the broadest sense and includes polyclonal and monoclonal antibodies, including intact antibodies and functional (antigen-binding) antibody fragments thereof, including fragment antigen binding (Fab) fragments, F(ab′) 2 fragments, Fab′ fragments, Fv fragments, recombinant IgG (rlgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “blood-related sample” refers to blood samples including whole-blood samples as well as samples derived from whole blood by the addition or removal of one or more cell types or chemical or biological molecules.

The term “apheresis” refers to a procedure in which blood from a patient or donor is at least partially separated from some of its components. More specific terms are “plateletpheresis” (referring to the separation of platelets) and “leukapheresis” (referring to the separation of leukocytes). In this context, the term “separation” refers to the obtaining of a product that is enriched in a particular component compared to whole blood and does not mean that absolute purity has been attained.

The term “T cell” refers to a subset of lymphocytic cells that are present in PBMC and express a surface marker of “CD3” (T-cell receptor). Unless otherwise indicated T cells are intended to include CD4+ (i.e., T-helper cells) and CD8+ (i.e., cytotoxic killer cells).

As used herein “genetically engineered” and grammatical equivalents refer to the modification of a cell with one or more exogenous nucleic acids and confers modified, additional or different functionality to the cell. For example, a genetically engineered cell may express a polypeptide form an exogenous nucleic acid source useful for therapeutic or research purposes. Alternatively, a genetically engineered cell may comprise one or more modifications that alter the nuclear DNA of the cell such as can be achieved by gene editing systems (e.g., TALEN or CRISPR systems), such modifications encompass deletions of, insertions to, or alterations of an existing nuclear DNA sequence.

The term “exogenous” refers to a substance or molecule originating or produced outside of an organism or cell. “exogenous” can also refer to the presence (e.g. protein, mRNA, transgene, etc.) of a molecule in cell, wherein the cell generally does not comprise the presence of the molecule. The term “exogenous gene” or “exogenous nucleic acid molecule,” as used herein, refers to a nucleic acid that codes for the expression of an RNA and/or protein that has been introduced (e.g. transformed or transfected) into a cell. An exogenous gene can be from a different species (and so a “heterologous” gene) or from the same species (and so a “homologous” gene), relative to the cell being transformed. An “exogenous polypeptide,” or “exogenous protein,” refers to a polypeptide chain produced by an exogenous nucleic acid of a cell or the cell comprising the exogenous nucleic acid that is not normally expressed by the cell.

The polypeptides described herein can be encoded by an exogenous nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements. The polypeptides may also be encoded by an exogenous RNA molecule.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The terms “enrich,” “isolate” and “purify” unless otherwise indicated, are synonymous and refer to the enrichment of a desired product relative to unwanted material. The terms do not necessarily mean that the product is completely isolated or completely pure. For example, if a starting sample had a target cell that constituted 2% of the cells in a sample, and a procedure was performed that resulted in a composition in which the target cell was 60% of the cells present, the procedure would have succeeded in enriching, isolating or purifying the target cell.

The terms “obstacle array”, “DLD array”, and “array” are used synonymously herein and describe an ordered array of obstacles that are disposed in a flow channel through which a cell or particle-bearing fluid can be passed. An obstacle array comprises a plurality of obstacles arranged in a column (along the path of fluid flow). Gaps between the obstacles (along the path of the fluid flow) allow the passage of cells or other particles. Such obstacles or columns can be arranged into one or more repeating rows (perpendicular to the path of fluid flow).

As described herein a “channel” or “lane” refers to a discreet separation unit with a plurality of obstacles that may be bounded on either side by walls such that discreet lanes are separated. Channels may run in parallel from one or more common inputs to one or more common outputs. Channels may be fluidly connected in series.

As described herein, the terms “fluid flow” and “bulk fluid flow” as used herein in connection with DLD refer to the macroscopic movement of fluid in a general direction across an obstacle array. These terms do not take into account the temporary displacements of fluid streams for fluid to move around an obstacle in order for the fluid to continue to move in the general direction.

As described herein, the term “tilt angle” or “E”: is the angle between the direction of bulk fluid flow and the direction defined by alignment of rows of sequential obstacles in an obstacle array.

As described herein, the term “array direction” is a direction defined by the alignment of rows of sequential obstacles in an obstacle array. A particle is “deflected” or “bumped” in an obstacle array if, upon passing through a gap and encountering a downstream obstacle, the particle's overall trajectory follows the direction of the columns of the obstacle array (i.e., travels at the tilt angle c relative to bulk fluid flow). A particle is not bumped if its overall trajectory follows the direction of bulk fluid flow under those circumstances.

The term “Deterministic Lateral Displacement” or “DLD” refers to a process in which particles are deflected on a path through an array, deterministically, based on their size in relation to some of the array parameters. This process can be used to separate cells, which is generally the context in which it is discussed herein. However, it is important to recognize that DLD can also be used to concentrate cells and for buffer exchange. Processes are generally described herein in terms of continuous flow (DC conditions; i.e., bulk fluid flow in only a single direction). However, DLD can also work under oscillatory flow (AC conditions; i.e., bulk fluid flow alternating between two directions).

The “critical size” or “predetermined size,” “critical diameter” or “predetermined diameter” of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however, when the profile is asymmetrical, the critical sizes of the two sides of the gap can differ.

The term “density gradient” with respect to a method of enrichment refers to the process of applying a force to cells that are suspended in a fluid or medium of a predetermined density such that cells travel though the fluid or medium based upon their density. The force is often applied by centrifugation, but can also be applied by pressure or other means that results in directional force being applied to the cells. Density gradient methods are often applied in circumstances where a heterogenous cell population exists and the cells differ based on their density such that at least two populations of cells can be separated. Cells can differ in density due to different activation states, viability (e.g., live/dead, necrotic, apoptotic), or different lineages (e.g., red blood cells vs. lymphocytes, platelets vs. red blood cells, non-nucleated cells vs. nucleated cells, etc.). “Density gradient medium or media” and grammatical equivalents refer to a fluid media of a predetermined density, including without limitation Percoll®, Histopaque®, or Ficoll. Density gradient media applied to cells is generally iso-osmotic and possess a density greater than water (1.0 grams per milliliter). Density gradient media may comprise a density of 1.05, 1.1, 1.2, 1.3, 1.4, or 1.5 grams per milliliter or greater. Density gradient media does not comprise water or water-based buffers or growth media that are substantially the same density as water. The density of a density gradient media may be the same throughout, separated into layers of distinct densities, or comprise a true gradient, where the density increases or decreases uniformly in a direction away from an applied force.

In certain embodiments, described herein, is a master cell bank comprising: (a) a population of enriched target cells comprising an exogenous nucleic acid described herein integrated at a genomic location or maintained episomally; and (b) a cryoprotectant. In certain embodiments, the cryoprotectant comprises glycerol or DMSO. In certain embodiments, the master cell bank is contained in a suitable vial or container able to withstand freezing by liquid nitrogen.

### Biological Samples

The methods and compositions described herein begin with the enrichment or particular target cells from a biological sample. Such biological samples can be from a mammalian source. In certain embodiments the biological samples are from a human source. The source can be from a single individual or a pooled from several individuals. In certain embodiments, the source for the biological sample is a single human individual. In certain embodiments, the source for the biological sample is a healthy individual. In certain embodiments, the source for the biological sample is an individual who has a cancer. In certain embodiments, the source for the biological sample is an individual who has leukemia.

In certain embodiments, the biological sample is a blood related product. Such blood related products can comprise whole blood or whole blood with one or more cells or serum components enriched or removed. In certain embodiments, the biological sample is a blood sample. In certain embodiments, the biological sample is an apheresis product. In certain embodiments, the biological sample is a leukapheresis product. A leukapheresis product is a product that has been enriched for leukocytes of lymphocyte and/or myeloid origin. Additionally, leukapheresis products may have reduced numbers of red blood cells and/or platelets. In certain embodiments, the biological sample is a leukopak. In certain embodiments, the biological sample is residual leukocytes from a platelet donation.

The sample may comprise a volume of at least about 50 mL, 100 mL, 200 mL, 300 mL, 400 ml, or 500 mL. A leukapheresis sample may comprise a volume of at least about 50 mL, 100 mL, 200 mL, 300 mL, 400 ml, or 500 mL.

In some embodiments, the methods begin with a biological sample comprising a certain hematocrit. Hematocrit is the percentage by volume of red blood cells (RBCs) in a sample, such as a blood sample comprising target cells and red blood cells (RBCs). A hematocrit can range from about 0.5% to about 50%. In some embodiments, the methods begin with a biological sample with a hematocrit percentage of red blood cells (RBCs) in a sample comprising target cells and red blood cells (RBCs) of about 0.5% to about 1%, about 0.5% to about 5%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.5% to about 45%, about 0.5% to about 50%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the methods yield a hematocrit percentage of red blood cells (RBCs) in a sample consisting of target cells and red blood cells (RBCs) of about 0.5%, about 1%, about 5%, about 10%, about 15%, about 20%, about 25%, about 30%, about 35%, about 40%, about 45%, or about 50%. In some embodiments, the methods yield a hematocrit percentage of red blood cells (RBCs) in a sample comprising target cells and red blood cells (RBCs) of at least about 0.5%, about 1%, about 2%, about 3%, about 4%, about 5%, about 10%, about 15%, about 20%, about 25%, about 30%, about 35%, about 40%, or about 45%. In some embodiments, maintaining an effective hematocrit can be achieved by adding red blood cells to a composition of target cells to achieved or produce the effective ratio.

Biological samples for the methods and composition described herein may also comprise bone marrow or adipose tissue obtained from a mammalian source. In certain embodiments, the mammalian source is a human source.

For example, some therapeutic applications such as CAR cell therapy or adoptive T cell therapies the sample may be an autologous sample for an individual to be treated. Also contemplated are blood related samples from an individual ultimately to be treated with a stem cell transplant or therapeutic cell. Also contemplated are samples from a family member, monozygotic twin, or otherwise HLA matched donor, providing cells for the therapeutic treatment of another individual (e.g., heterologous samples).

A sample for processing may have been subjected to one or more steps to prepare the sample for processing or to facilitate collection of the sample or make it suitable for separation, including the addition of anti-coagulants or the depletion of one or more non-target cells. Suitable anticoagulants include citric acid, sodium citrate, dextrose, heparin, and chelating agents such as EDTA or EGTA. In certain embodiments, the sample may be treated with anti-coagulant citrate dextrose solution (ACD-A, citric acid monohydrate, dextrose monohydrate, and trisodium citrate dihydrate). Individuals from which the sample is collected may be administered a blood thinner, anti-coagulant, or anti-inflammatory drug before collection.

### Size Based Enrichment

The methods described herein involve enriching cell populations based on a predetermined size resulting in an enriched cell population, wherein the enriched target cell population comprises cells above a certain size. When processing a blood-related sample cellular components that are smaller than red blood cells (e.g., less than about 7 micrometers or less than about 4 micrometers) cells can be specifically removed from a sample along with serum biomolecules. When processing a blood-related sample cellular components that are smaller than resting or activated leukocyte cells can be specifically removed from a sample along with serum biomolecules. In certain embodiments, cells less than about 11 micrometers are removed from the sample. In certain embodiments, cells less than about 10 micrometers are removed from the sample. In certain embodiments, cells less than about 9 micrometers are removed from the sample. In certain embodiments, cells less than about 8 micrometers are removed from the sample. In certain embodiments, cells less than about 7 micrometers are removed from the sample. In certain embodiments, cells less than about 6 micrometers are removed from the sample. In certain embodiments, cells less than about 5 micrometers are removed from the sample. In certain embodiments, cells less than about 4 micrometers are removed from the sample. In certain embodiments, cells less than about 3 micrometers are removed from the sample.

In certain embodiments, cells less than about 11 micrometers are removed while cells greater than about 11 micrometers are preserved. In certain embodiments, cells less than about 10 micrometers are removed while cells greater than about 10 micrometers are preserved. In certain embodiments, cells less than about 9 micrometers are removed while cells greater than 9 micrometers are preserved. In certain embodiments, cells less than about 8 micrometers are removed while cells greater than 8 micrometers are preserved. In certain embodiments, cells less than about 7 micrometers are removed while cells greater than 7 micrometers are preserved. In certain embodiments, cells less than about 6 micrometers are removed while cells greater than 6 micrometers are preserved. In certain embodiments, cells less than about 5 micrometers are removed while cells greater than 5 micrometers are preserved. In certain embodiments, cells less than about 4 micrometers are removed while cells greater than 4 micrometers are preserved. In certain embodiments, cells less than about 3 micrometers are removed while cells greater than 3 micrometers are preserved.

### Density Based Enrichment

The methods described herein involve enriching cell populations based on a density cutoff resulting in an enriched target cell population, wherein the enriched target cell population comprises cells above a certain density. When processing a blood-related sample cellular components that are smaller than red blood cells (e.g., less than about 1.11 grams per milliliter) cells can be specifically removed from a sample along with serum biomolecules. In certain embodiments, cells less than about 1.11 g/mL are removed from the sample. In certain embodiments, cells less than about 1.10 g/mL micrometers are removed from the sample. In certain embodiments, cells less than about 1.09 g/mL micrometers are removed from the sample. In certain embodiments, cells less than about 1.08 g/mL micrometers are removed from the sample. In certain embodiments, cells less than about 1.07 g/mL micrometers are removed from the sample.

In certain embodiments, cells less than about 1.11 g/mL are removed while cells greater than 1.11 g/mL are preserved. In certain embodiments, cells less than 1.10 g/mL are removed while cells greater than 1.10 g/mL are preserved. In certain embodiments, cells less than 1.09 g/mL are removed while cells greater than 1.09 g/mL are preserved. In certain embodiments, cells less than 1.08 g/mL are removed while cells greater than 1.08 g/mL are preserved. In certain embodiments, cells less than 1.07 g/mL are removed while cells greater than 1.07 g/mL are preserved.

### Enriched Cell Populations

The methods described herein produce compositions of enriched target cells. In certain embodiments, an enriched target cell population comprises a population of target cells and red blood cells, wherein the target cells and blood cells comprise greater than about 75%, 80%, 85%, 90%, 95%, 97%, 98%, or 99% of the enriched target cell population. In certain embodiments, an enriched target cell population comprises a population of lymphocyte cells and red blood cells, wherein the lymphocyte cells and blood cells comprise greater than about 75%, 80%, 85%, 90%, 95%, 97%, 98%, or 99% of the enriched target cell population. In certain embodiments, an enriched target cell population comprises a population of T cells and red blood cells, wherein the T cells and blood cells comprise greater than about 75%, 80%, 85%, 90%, 95%, 97%, 98%, or 99% of the enriched target cell population. In certain embodiments, the enriched target cell population is substantially free of platelets. In certain embodiments, the enriched target cell population comprises less than about 10%, 5%, 4%, 3%, 2%, or 1% platelets. In certain embodiments, the enriched target cell population is substantially free of red blood cells. In certain embodiments, the enriched target cell population comprises less than about 10%, 5%, 4%, 3%, 2%, or 1% red blood cells. In certain embodiments, the enriched target cell population comprises activated T cells. In certain embodiments, the enriched target cell population comprises naive T cells. In certain embodiments, the enriched target cell population comprises resting or unactivated T cells. In certain embodiments, the enriched target cell population comprises central memory (CD62L+) T cells. In certain embodiments, the enriched target cell population comprises or consists of human cells.

In certain embodiments, an enriched target cell population comprises a population of target cells and red blood cells, wherein the target cells comprise greater than about 50%, 55%, 60%, 65%, 70%, 75%, 80%, 85%, 90%, 95%, 97%, 98%, or 99% of the enriched target cell population. In certain embodiments, an enriched target cell population comprises a population of lymphocyte cells and red blood cells, wherein the lymphocyte cells comprise greater than about 50%, 55%, 60%, 65%, 70%, 75%, 80%, 85%, 90%, 95%, 97%, 98%, or 99% of the enriched target cell population. In certain embodiments, an enriched target cell population comprises a population of T cells and red blood cells, wherein the T cells comprise greater than about 50%, 55%, 60%, 65%, 70%, 75%, 80%, 85%, 90%, 95%, 97%, 98%, or 99% of the enriched target cell population. In certain embodiments, the enriched target cell population is substantially free of platelets. In certain embodiments, the enriched target cell population comprises less than about 10%, 5%, 4%, 3%, 2%, or 1% platelets. In certain embodiments, the enriched target cell population comprises activated T cells. In certain embodiments, the enriched target cell population comprises naive T cells. In certain embodiments, the enriched target cell population comprises central memory (CD62L+) T cells. In certain embodiments, the enriched target cell population comprises or consists of human cells.

The enriched target cell populations can comprise an exogenous nucleic acid. The exogenous nucleic acid can comprise a coding region for a polypeptide, optionally a non-viral polypeptide. In certain embodiments, greater than about 50%, 55%, 60%, 65%, 70%, 75%, 80%, 85%, 90%, or 95% of the of the enriched target cells comprise an exogenous nucleic acid. In certain embodiments, greater than about 50%, 55%, 60%, 65%, 70%, 75%, 80%, 85%, 90%, or 95% of the of the lymphocyte cells comprise an exogenous nucleic acid. In certain embodiments, greater than about 50%, 55%, 60%, 65%, 70%, 75%, 80%, 85%, 90%, or 95% of the of the T cells comprise an exogenous nucleic acid. In certain embodiments, the polypeptide encoded by the exogenous nucleic acid comprises an immunoglobulin, a chimeric antigen receptor, a T cell receptor, a cytokine, or a chemokine. In certain embodiments, the polypeptide encoded by the exogenous nucleic acid comprises a chimeric antigen receptor.

The enriched target cell populations may further comprise cytokines, chemokine or growth factors that support cell growth and division. In certain embodiments, the cell populations comprise any one or more of IL-15, IL-7, anti-CD28 antibody, or anti-CD3 antibody. In certain embodiments, the cell population comprises at least about 5 ng/ml, 10 ng/ml, 20 ng/ml, 30 ng/ml, 40 ng/mL, 50 ng/ml of IL-15. In certain embodiments, the cell population comprises at least about 5 ng/ml, 10 ng/ml, 20 ng/mL, 30 ng/ml, 40 ng/mL, 50 ng/ml of IL-17. The IL-15 or IL-7 may be recombinant human IL-15 or IL-7.

### Enrichment Methods

### Density Gradient Separation

Methods comprising centrifugal apheresis separates the plasma from cellular components based on density can be useful for obtaining one or more target cells from a blood related sample. Density gradient separation apheresis devices are designed to separate plasma or blood components from whole blood, for the purposes of depletion or exchange of these components or plasma. Density gradient separation comprises drawing whole blood from a patient and separating the blood into its components, utilizing centrifugal force as the basis of operation. Centrifugal flow devices most commonly deliver continuous flow from the patient to the centrifuge. An anticoagulant, usually citrate, is added before centrifugation, which is then followed by return of the rest of the blood components with the appropriate replacement fluid (typically albumin or plasma) so that a continuous flow extracorporeal circuit is formed.

Accordingly, density gradient separation can be used for generating a population of enriched target cells from a sample. In certain embodiments, the population of enriched target cells comprises a ratio of platelets to target cells of less than about 500:1. In certain embodiments, the population of enriched target cells comprises a ratio of platelets to target cells of less than about 100:1. In certain embodiments, the population of enriched target cells comprises a ratio of platelets to target cells of less than about 10:1. In certain embodiments, the population of enriched target cells comprises a ratio of platelets to target cells of less than about 5:1. In certain embodiments, the population of enriched target cells comprises a ratio of red blood cells to target cells of greater than about 100:1. In certain embodiments, the population of enriched target cells comprises a ratio of red blood cells to target cells of greater than about 250:1. In certain embodiments, the population of enriched target cells comprises a ratio of red blood cells to target cells of greater than about 500:1. In certain embodiments, the population of enriched target cells comprises a ratio of red blood cells to target cells of no greater than about 1,000:1. In some embodiments, density gradient separation is used for the isolation of lymphocytes. In some embodiments, density gradient separation is used for the isolation of hematopoietic stem cells. In some embodiments, density gradient separation is used for the isolation of mesenchymal stem cells. In certain embodiments, the isolation of peripheral blood mononuclear cells (PBMCs) is used for the isolation of T cells for the generation of chimeric antigen receptor T cells (CAR-T cells).

### Array-Based Separation

Methods utilizing arrays comprising microstructures (e.g. microposts or columns) that construct pores that separate cells based on critical sizes. For example, such methods generally utilize size exclusion to prevent or restrict entrance or passage by physical blockage. Embodiments of size exclusion comprise the use of small pores to prevent large non-deformable particles from entering the pores. The pore size can be engineered to allow for the separation of particles of different sizes (critical sizes). Such methods can also utilize laminar flow, tangential flow, or cross flow dynamics to facilitate sample processing. Accordingly, density gradient separation can be used for generating the target cell compositions disclosed herein.

For example, methods comprising Deterministic Lateral Displacement (DLD) for separating different cell types can be useful for obtaining one or more target cells from a blood related sample without performing density gradient centrifugation. See Campos-Gonzalez et al. (2018). Deterministic Lateral Displacement: The Next-Generation CAR T-Cell Processing?, SLAS Technology, 23 (4), 338-351. DOI: 10.1177/2472630317751214, which is hereby incorporated by reference in its entirety. DLD is a process in which particles are deflected on a path through an array in a microfluidic device, deterministically, based on their size in relation to some of the array parameters. The microfluidic device used in DLD comprises channels with arrays of posts having a diameter of about 20 micrometers. The device may and contain at least 10, 15, 20, 25, or 50 channels. DLD can also be used to concentrate cells and for buffer exchange. Processes are generally described herein in terms of continuous flow (DC conditions; i.e., bulk fluid flow in only a single direction). However, DLD can also work under oscillatory flow (AC conditions; i.e., bulk fluid flow alternating between two directions). DLD generally functions to separate cells or components thereof base on the critical size or predetermined size of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however, when the profile is asymmetrical, the critical sizes of the two sides of the gap can differ.

As described herein a critical size applied to a method of separating cells for transfection can comprise about 2 micrometers, about 3 micrometers, about 3.4 micrometers, about 3.5 micrometers, about 3.6 micrometers, about 3.7 micrometers, about 3.8 micrometers, about 4 micrometers, about 5 micrometers about 6 micrometers, about 7 micrometers, about 8 micrometers or greater. The critical size may differ from the actual size of the cells that are being separated as flow through a microfluidic device may make cells appear larger or smaller depending upon variables such as tonicity of the separation media, flow rate, and other factors that may affect the apparent or hydrodynamic size of the cells being separated.

Basic principles of size based microfluidic separations and the design of obstacle arrays for separating cells have been provided elsewhere (see, US 2014/0342375; US 2016/0139012; U.S. Pat. Nos. 7,318,902 and 7,150,812, which are hereby incorporated herein in their entirety) and are also summarized in the sections below.

Procedures for making and using microfluidic devices that are capable of separating cells on the basis of size have also been described in the art. Such devices include those described in U.S. Pat. Nos. 5,837,115; 7,150,812; 6,685,841; 7,318,902; 7,472,794; and 7,735,652; all of which are hereby incorporated by reference in their entirety. Other references that provide guidance that may be helpful in the making and use of devices for the present invention include: U.S. Pat. Nos. 5,427,663; 7,276,170; 6,913,697; 7,988,840; 8,021,614; 8,282,799; 8,304,230; 8,579,117; US 2006/0134599; US 2007/0160503; US20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 all of which are also incorporated by reference herein in their entirety.

Described herein are examples of a microfluidic device for separating target particles or target cells of a predetermined size from other constituents of a sample. The device may have a planar support that will typically be rectangular and can be made of any material compatible with a separation method, including silicon, glasses, hybrid materials or (preferably) polymers. The support may have a top surface and a bottom surface, one or both of which have at least one embedded channel extending from one or more sample inlets and one or more distinct fluid inlets, to one or more product outlets and one or more distinct waste outlets. Fluid inlets (as opposed to sample inlets) may sometimes be referred to as “buffer” or “wash” inlets and, depending on the objectives of a separation may be used to transport a variety of fluids into channels. Unless otherwise indicated by usage or context, it will be understood that a “fluid” may be a buffer, contain reagents, constitute growth medium for cells or generally be any liquid, and contain any components, compatible with operation of a device and the objectives of the user.

When fluid is applied to a device through a sample or fluid inlet, it flows through the channel toward the outlets, thereby defining a direction of bulk fluid flow. In order to separate cells or particles of different sizes, the channel includes an array of obstacles organized into columns that extend longitudinally along the channel (from inlet to outlet), and rows that extend laterally across the channel. Each subsequent row of obstacles is shifted laterally with respect to the previous row, thereby defining an array direction that deviates from the direction of bulk fluid flow by a tilt angle (c). The obstacles are positioned so as to define a critical size such that when a sample is applied to an inlet of the device and flows to an outlet, particles or cells in the sample larger than the critical size follow in the array direction and particles smaller than the critical size flow the direction of bulk fluid flow, thereby resulting in a separation.

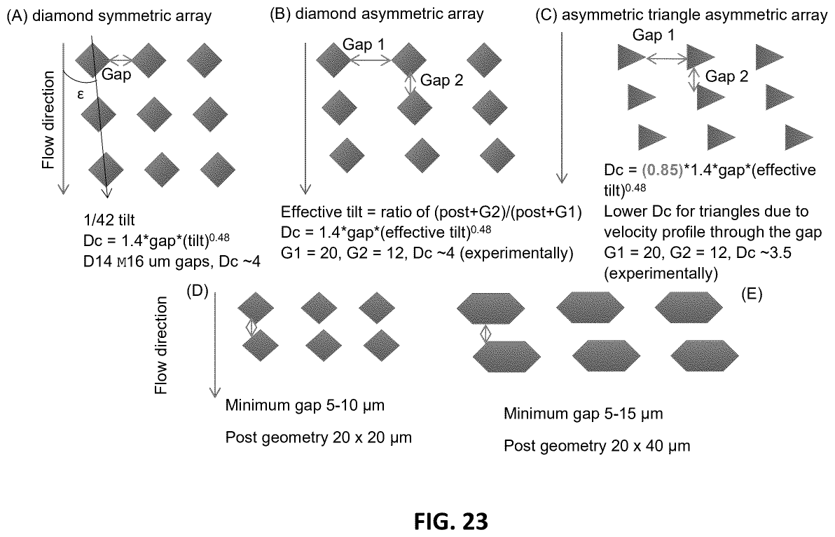

Adjacent obstacles in a row of the array are separated by a gap, G1, that is perpendicular to the direction of bulk fluid flow and adjacent obstacles in a column are separated by a gap, G2, which is parallel to the direction of bulk fluid flow (see FIGS. 23A and 23B). One characteristic of the present devices is that the ratio of the size of gap G2 to the size of gap G1 does not equal 1, with G1 typically being wider than G2 (e.g., by 10-100%). The obstacles in an array each have at least two vertices and are positioned so that each gap is flanked on either side by at least one vertex. In preferred embodiments, the vertices extend into parallel gaps so that the gaps are flanked on either side by one or more vertices pointing toward one another but not directly opposite one another and/or obstacles have vertices that extend into perpendicular gaps such that the gaps are flanked on either side by vertices pointing toward, and directly opposite to, one another (see FIGS. 23A and 23B).

In some embodiments, G1 and G2 may each independently be about 9 μm to about 30 μm. In some embodiments, G1 and G2 may each independently be about 9 μm to about 11 μm, about 9 μm to about 13 μm, about 9 μm to about 15 μm, about 9 μm to about 17 μm, about 9 μm to about 19 μm, about 9 μm to about 21 μm, about 9 μm to about 22 μm, about 9 μm to about 24 μm, about 9 μm to about 26 μm, about 9 μm to about 28 μm, about 9 μm to about 30 μm, about 11 μm to about 13 μm, about 11 μm to about 15 μm, about 11 μm to about 17 μm, about 11 μm to about 19 μm, about 11 μm to about 21 μm, about 11 μm to about 22 μm, about 11 μm to about 24 μm, about 11 μm to about 26 μm, about 11 μm to about 28 μm, about 11 μm to about 30 μm, about 13 μm to about 15 μm, about 13 μm to about 17 μm, about 13 μm to about 19 μm, about 13 μm to about 21 μm, about 13 μm to about 22 μm, about 13 μm to about 24 μm, about 13 μm to about 26 μm, about 13 μm to about 28 μm, about 13 μm to about 30 μm, about 15 μm to about 17 μm, about 15 μm to about 19 μm, about 15 μm to about 21 μm, about 15 μm to about 22 μm, about 15 μm to about 24 μm, about 15 μm to about 26 μm, about 15 μm to about 28 μm, about 15 μm to about 30 μm, about 17 μm to about 19 μm, about 17 μm to about 21 μm, about 17 μm to about 22 μm, about 17 μm to about 24 μm, about 17 μm to about 26 μm, about 17 μm to about 28 μm, about 17 μm to about 30 μm, about 19 μm to about 21 μm, about 19 μm to about 22 μm, about 19 μm to about 24 μm, about 19 μm to about 26 μm, about 19 μm to about 28 μm, about 19 μm to about 30 μm, about 21 μm to about 22 μm, about 21 μm to about 24 μm, about 21 μm to about 26 μm, about 21 μm to about 28 μm, about 21 μm to about 30 μm, about 22 μm to about 24 μm, about 22 μm to about 26 μm, about 22 μm to about 28 μm, about 22 μm to about 30 μm, about 24 μm to about 26 μm, about 24 μm to about 28 μm, about 24 μm to about 30 μm, about 26 μm to about 28 μm, about 26 μm to about 30 μm, or about 28 μm to about 30 μm. In some embodiments, G1 and G2 may each independently be about 9 μm, about 11 μm, about 13 μm, about 15 μm, about 17 μm, about 19 μm, about 21 μm, about 22 μm, about 24 μm, about 26 μm, about 28 μm, or about 30 μm. In some embodiments, G1 and G2 may each independently be at least about 9 μm, about 11 μm, about 13 μm, about 15 μm, about 17 μm, about 19 μm, about 21 μm, about 22 μm, about 24 μm, about 26 μm, or about 28 μm. In some embodiments, G1 and G2 may each independently be at most about 11 μm, about 13 μm, about 15 μm, about 17 μm, about 19 μm, about 21 μm, about 22 μm, about 24 μm, about 26 μm, about 28 μm, or about 30 μm.

The microfluidic devices will also typically have an obstacle bonding layer that is bonded to a surface of the planar support and bonded to the obstacles in channels to prevent fluid or sample from flowing over obstacles during operation of the device. This obstacle bonding layer may comprise one or more passages fluidically connected to the inlets of the channel and to the outlets of the channel which permit the flow of fluid.

In general, the microfluidic devices will be used to separate target particles or target cells having a size larger than the critical size of the device from contaminants, fluids, non-target particles, or non-target cells with sizes smaller than the critical size. When a sample containing the target cells or particles is applied to a device through a sample inlet and fluidically passed through the channel, the target cells or target particles will flow to one or more product outlets where a product enriched in target cells or target particles is obtained. The term “enriched” as used in this context means that the ratio of target cells or particles to contaminants is higher in the product than in the sample. Contaminants, fluids, non-target particles, and non-target cells with a size smaller than the critical size will flow predominantly to one more waste outlets where they may be either collected or discarded.

Although the objective of a separation will generally be to separate target cells or particles from smaller contaminants, there may be times when a user wants to separate target cells or particles from larger contaminants. In these instances, a microfluidic device may be used with a critical size larger than the target cells or particles but smaller than the contaminants. Combinations of two or more obstacle arrays with different critical sizes, either on a single device or on multiple devices, may also be used in separations. For example, a device may have channels with a first array of obstacles that has a critical size larger than T cells but smaller than granulocytes and monocytes and a second array with a critical size smaller than T cells but larger than platelets and red blood cells. Processing of a blood sample on such a device allows for the collection of a product in which T cells have been separated from granulocytes, monocytes, platelets and red blood cells. The order of the obstacle arrays should not be of major importance to the result, i.e., an array with a smaller critical size could come before or after an array with a larger critical size. Also arrays with different critical sizes can be on separate devices that cells pass through.

Wide arrays and multiple outlets may be used for the collection multiple products, e.g., monocytes may be obtained at one outlet and T cells at a different outlet. Thus, using multiple arrays and multiple outlets may permit the concurrent collection of several products that are more purified than if a single array had been used. As further discussed below, high throughputs may be maintained by using many DLD arrays in parallel.

Preferably, the obstacles used in the microfluidic devices have a polygonal shape, with diamond or hexagonally shaped obstacles being preferred. The obstacles will also generally be elongated so that their length perpendicular to bulk fluid flow (P1) is different (generally longer) than their width parallel to bulk fluid flow (P2) by, for example, 10-100% (see FIG. 23B). Typically, P1 will be longer than P2 by at least 15%, 30%, 50%, 100% or 150%. Expressed as a range, P1 may be 10-150% (15-100%; or 20-70%) longer than P2.

Microfluidic devices may also include a separator wall that extends from the sample inlet of a device, where it separates the sample inlet from fluid inlets and prevents mixing, into the array of obstacles in the channel. The separator wall is oriented parallel to the direction of bulk fluid flow and extends toward the sample and fluid outlets. The wall terminates before reaching the end of the channel, allowing sample and fluid streams to contact one another thereafter. It should generally extend at for a distance of at least 10% of the length of the array of obstacles but may extend for at least 20%, 40%, 60%, or 70% of the array. Expressed as a range the wall will typically extend for 10-70% of the length of the array of obstacles. More than one separator wall may also be present in a device and, depending on the objectives of a separation, may be positioned in different ways.

In order to increase the rate at which volume can be processed, a stacked separation assembly can be made by overlaying a first obstacle array with one or more stacked obstacle arrays, wherein the bottom surface of each stacked array is in contact with either the top surface, or an obstacle bonding layer on the top surface, of the first obstacle array or with the top surface, or the obstacle bonding layer on the top surface, of another array. Sample is provided to the sample inlets of all devices though a first common manifold and fluid is supplied to the fluid inlets through a second manifold that may or may not be the same as the first manifold. Product is removed from the product outlets through one or more product conduits and waste is removed from the waste outlets through one or more waste conduits that are different from the product conduits. In general, a stacked separation assembly will have 2 to 9 stacked arrays together with the first microfluidic obstacle array. However, a larger number of devices may also be used. In addition, the top surface of supports, and/or the bottom surface, may have multiple (e.g., 2-40 or 2-30) embedded channels and be used in purifying target particles or target cells.

Stacked separation assemblies may have a reservoir bonding layer which is attached to the bottom surface of the first microfluidic device and/or to the top surface of a stacked microfluidic device. The reservoir bonding layer should include a first end with one or more passages permitting the flow of fluid to inlets on the channels and optionally, one or more passages that permit the flow of fluid to, or from, the product and waste outlets of channels at a second end, opposite to first end and separated by material impermeable to fluid.

Stacked assemblies of devices may be supported in a cassette characterized by the presence of an outside casing with ports allowing for the transport of sample and fluids into the cassette and products and waste out of the cassette. The figure shows a cassette with two inlet ports and two outlet ports. However, multiple ports into and out of a cassette may be used and several products may be collected essentially simultaneously. It will also be recognized that cassettes can be part of a system in which there are components that are well known and commonly used in the art. Such common components include, pumps, valves and processors for controlling fluid flow; sensors for monitoring system parameters such a flow rate and pressure; sensors for monitoring fluid characteristics such a pH or salinity; sensors for determining the concentration of cells or particles; and analyzers for determining the types of cells or particles present in the cassette or in material collected from the cassette. More generally, any equipment known in the art and compatible with the cassettes, the material being processed, and the processing objectives may be used.

In another aspect, the invention is directed to a method for purifying target particles or target cells of a predetermined size from contaminants by obtaining a sample comprising the target particles or target cells and contaminants and carrying out a purification using any of the microfluidic devices or stacked separation assembles discussed herein. Purification is accomplished by applying the sample to one or more sample inlets on any of the microfluidic devices discussed above or to sample inlets on the first microfluidic device or a stacked device in an assembly of devices. A manifold may be used to apply sample to inlets, particularly when using stacked devices. Samples are then flowed through the channel to the outlets of devices. Generally, the target particles or target cells will have a size larger than the critical size of the array of obstacles on devices and at least some contaminants will have sizes smaller than the critical size. As a result, the target cells or target particles will flow to one or more product outlets where a product enriched in target cells or target particles is obtained and contaminants with a size smaller than the critical size will flow to one more waste outlets. As noted previously however, there may be instances where the target cells or target particles are smaller than contaminants and devices are chosen with a critical size larger than the target cells or particles and smaller than the contaminants. In these cases, the general operation of devices will be essentially the same but contaminants will flow in the array direction and target cells or particles will proceed in the direction of bulk fluid flow.

### Designing Microfluidic Cartridges

The present disclosure provides microfluidic cartridges (i.e. devices, chips, cassettes, plates, microfluidic devices, cartridges, DLD devices, etc.) for purifying particles or cells. A microfluidic cartridge of the present disclosure may operate using a DLD method. A microfluidic cartridge of the present disclosure may be formed from a polymeric materials (e.g. thermoplastic), and may include one or more of a first planar support having a top surface and a bottom surface, and a second planar support having a top surface and a bottom surface, wherein the top surface of the first and second planar support comprises at least one embedded channel extending from one or more inlets to one or more outlets; the at least one embedded channel comprising an array of obstacles, wherein the bottom surface of the first and second planar support comprises a void space configured to be deformed when a the bottom of the first planar support is pressed to the bottom of the second planar support. A microfluidic cartridge of the present disclosure may be a single-use or disposable device. As an alternative, the microfluidic cartridge may be multi-use device. The use of polymers (e.g., thermoplastics) to form the microfluidic structure may allow for the use of an inexpensive and highly scalable soft embossing process while the void space may provide an improved ability to be manufactured quickly and avoid damage to the obstacles (i.e. posts, DLD arrays, etc.) during the manufacturing process.

The cartridges described herein may operate via deterministic lateral displacement, or DLD. DLD may include three different operating modes. The operating modes include: i) Separation, ii) Buffer Exchange and iii) Concentration. In each mode, particles above a critical diameter are deflected in the direction of the array from the point of entry, resulting in size selection, buffer exchange or concentration as a function of the geometry of the array. In all cases, particles below the critical diameter pass directly through the device under laminar flow conditions and subsequently off the device. The full length of the separation zone of the microfluidic cartridge may be about 75 mm and the width may be about 40 mm, with each individual channel being about 1.8 mm across.

The cartridges described herein may be arranged in a variety of orientations to accomplish different DLD modes or product outcomes. For example, four channels with side walls and an array of obstacles may be utilized. Samples containing blood, cells or particles may enter the channel through a sample inlet at the top and buffer, reagent or media may enter the channel at a separate fluid inlet. As they flow toward the bottom of the channels, cells or particles with sizes larger than the critical diameter of the array (>DC) flow at angle that is determined by the array direction of the obstacles and are separated from cells and particles with sizes smaller than the critical diameter of the array (<DC).

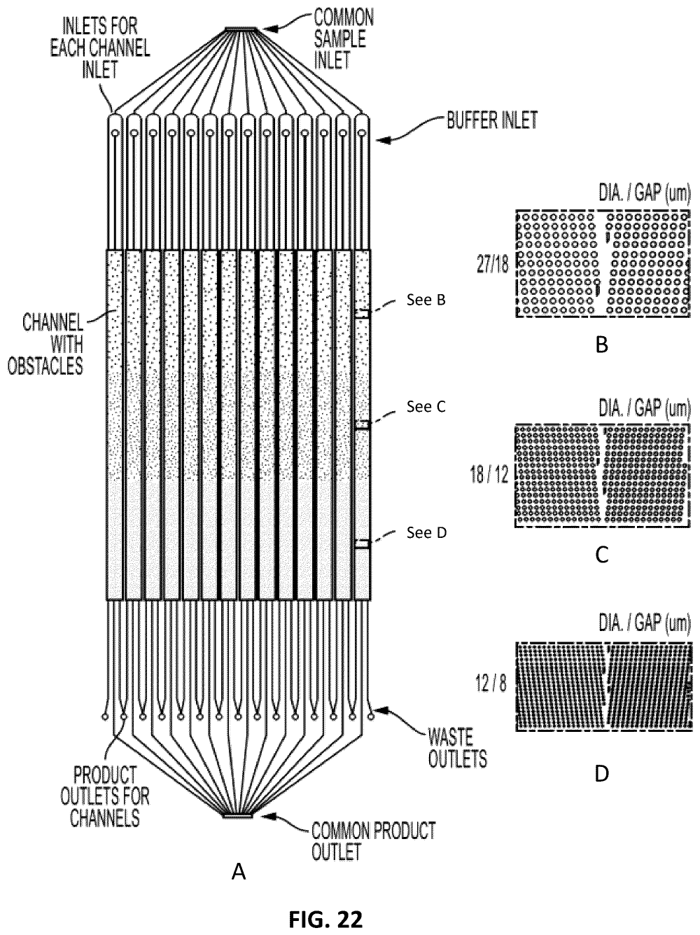

Referring to FIGS. 22A-22D, an embodiment of a cartridge may comprise an arrangement of 14 parallel channels that could be used in a microfluidic device or cartridge. FIGS. 22B-22D illustrate expanded views of sections of the cartridge. In this illustration, the channels have three zone (sections) with progressively smaller gaps. The cartridge has a common sample inlet, e.g., for blood, which feeds the sample to inlets on each channel. There are separate inlets into channels for buffer, but which could, depending processing objectives, be used to introduce fluids with reagents, growth medium or other fluids into channels. At the bottom of each channel there is a product outlet which would typically be used for recovering target cells or particles that have sizes larger than the critical diameter of the obstacle arrays in the channels. The outlets from the individual channels feed into a common product outlet from which the target cells or particles can be recovered. Also shown are waste outlets in which cells and particles with sizes below the critical diameter of the obstacle arrays in the channels exit.

An embodiment of a cartridge may comprise 2 channels. The channels may have three sections designed to have progressively smaller diameter obstacles and gaps. Some cartridges may have a “bump array” having equilateral triangularly shaped obstacles disposed in a microfluidic channel. Equilateral triangular posts may be disposed in a parallelogram lattice arrangement that is tilted with respect to the directions of fluid flow. Other lattice arrangements (e.g., square, rectangular, trapezoidal, hexagonal, etc. lattices) can also be used. The tilt angle (epsilon) is chosen so the device is periodic. In some embodiments, a tilt angle of 18.4 degrees (⅓ radian) makes the device periodic after three rows. The tilt angle E also represents the angle by which the array direction is offset from the fluid flow direction. The gap between posts is denoted G with equilateral triangle side length S. Streamlines extend between the posts, dividing the fluid flow between the posts into three regions (“stream tubes”) of equal volumetric flow. A relatively large particle (having a size greater than the critical size for the array) follows the array tilt angle when fluid flow is in the direction shown. A relatively small particle (having a size smaller than the critical size for the array) follows the direction of fluid flow.

The cartridges provided herein may comprise arrays of diamond shaped posts as illustrated in FIGS. 23A-23B. FIG. 23A shows a symmetric array of obstacles in which gaps perpendicular to the direction of fluid flow, e.g., Gap 1 (G1), and gaps parallel to the direction of fluid flow, e.g., Gap 2 (G2) are all about the same length. Diamond shaped obstacles may have two diameters, one perpendicular to the direction of fluid flow (P1) and the other parallel to the direction of fluid flow (P2). The right side of the figure shows an asymmetric array in which parallel gaps are shorter than perpendicular gaps. Although, G1 in the asymmetric array has been widened compared to the symmetric array, the reduction in gap G2 results in a critical diameter for the array that is the same as for the symmetrical array. As a result, the two arrays should be about equally effective at separating particles or cells of a given diameter in a sample. However, the widening of G1 allows for a higher sample throughput and reduces channel clogging. FIG. 23B shows, on the left side, an array of diamond obstacles that have been elongated so that their vertical diameter is longer than their horizontal diameter. The middle section of FIG. 23 shows diamond posts that have been elongated so that their horizontal diameter is longer than their vertical diameter and the far-right section of the figure shows hexagonally shaped obstacles that have been horizontally elongated.

Cartridges describe herein may comprise a stacked separation assembly in which two microfluidic devices or cartridges are combined into a single unit. The topmost device may comprise a planar support that may be made using a variety of materials, but which is most preferably polymeric and which has a top surface and a bottom surface. The top surface of the support may contain reservoirs that provide sample inlets and inlets for buffer or other fluid at one end of the support and product outlets and waste outlets at the other end. Each reservoir may be fluidically connected through the support using small vias that connect the top surface to the channels on the bottom surface. The bottom surface of the support may have numerous embedded microfluidic channels each of which may have an array of obstacles (see FIGS. 22B-22D, 23B, and 23B) connected by the channels. The embedded microfluidic layers may be bonded to an obstacle bonding layer that seals the first device and prevents fluid from flowing over the obstacles during operation. A second microfluidic device in the stack may contain embedded microfluidic channels on the topmost surface, and may be sealed by the same obstacle bonding layer as the topmost device. A reservoir bonding layer may have oblong openings allowing for the passage of liquid to channel inlets and the passage of liquid from channel outlets. The reservoir bonding layer may be similar to the obstacle bonding layer except that it attaches to a surface of a device and not obstacles and may be connected to one or more reservoirs feeding the stack of devices or to a manifold. Holes may be used for aligning the stacked devices. As described above, the two embedded microfluidic surfaces may face the same obstacle bonding layer. An alternate configuration would be to have the embedded channels on the top surface of both devices, with an intermediate layer between the devices that functions as both an obstacle bonding layer to the embedded channels below and a distribution layer to the reservoirs above. Multiple microfluidic devices may be stacked together to form a single assembly unit. At the top of this stack (and optionally both at the top and bottom) may be a manifold with feeds for a manifold inlet distributor and conduits leading from the manifold product outlet. Feeds leading to fluid inlets and conduits for removing fluid from waste outlets may also be present.

In certain examples, a device may have two channels where each channel has an array of asymmetrically spaced diamond obstacles, in which G1 is larger than G2. The diamonds may be offset so each successive row is shifted laterally relative to the previous row.

The present disclosure provides herein stacked assemblies of microfluidic devices inside a casing which together may be referred to as “cassettes” or a “cassette”. A port may serve as a feed for sample being fed through the casing and to a manifold. The port may be connected to manifold feeds which distribute sample through a manifold sample inlet to channel sample inlets. Once applied, sample flows through channels containing obstacle arrays (see FIGS. 22, 23) and product having particles or cells larger than the critical size exit the stack of devices at a manifold product outlet. The product then flows from the manifold outlet through product conduits and is conveyed out of the cassette through product outlet port. Fluid flows into the cassette and to the manifold through port, which is connected to manifold fluid feeds. It may be distributed by a manifold fluid inlet to channel fluid inlets. The fluid flows through the channel and particles or cells smaller than the critical size exit the stack of devices predominantly through manifold waste outlet. These particles or cells then flow through waste conduits that convey waste out of the cassette through outlet port.

An embodiment of the cartridges or devices provided herein may comprise a channel bounded by two walls with a sample inlet and a fluid inlet. There may be a separator wall that prevents the sample flow stream from mixing with the fluid flow stream. The separator wall may extend into the obstacle array and end about halfway down. Initially after entering the obstacle array, the target cells may be diverted away from the direction of fluid flow until they reach the separator wall. They may then travel along the wall until it ends. Thereafter, they may resume being diverted until they exit the channel at the product outlet. Particles with sizes smaller than the critical size of the obstacle array are not diverted and exit the channel at the waste outlet. A channel may be bounded by walls with an inlet for sample, an inlet for a reagent and an inlet for buffer or other fluid. Sample may enter at the inlet and flow onto the obstacle array. There, particles or cells larger than the critical diameter of the array are diverted into the reagent stream where they undergo a reaction. A separator wall may run from the reagent inlet part way down the array of obstacles and may separate the reagent stream from the stream of buffer or other fluid. This wall maintains the cells or particles in the reagent stream for a longer period of time, thereby providing more time for reaction. At the end of the separator wall, the particles or cells resume being diverted to a product outlet where they may be collected. During this process the cells or particles are separated from unreacted reagent. A second separator wall may run from the end of the first separator wall to a waste outlet where buffer or other fluid, reagent and small particles or cells exit the device and may be collected or discarded. A second waste outlet may be used to remove reagent, fluid in which particles or cells in the sample were suspended and particles or cells smaller than the critical diameter of the obstacle array. These materials may be recovered or discarded.

G_{T} refers to the gap length between triangular posts, and G_{C} refers to the gap length between round posts. As the array tilt increases, the difference in gap lengths required for a particular critical size of the array (D_{C}), between triangular and circular posts, decreases.

The obstacle edge roundness (expressed as r/S) may have an effect on the critical size exhibited on the side of a gap bounded by the edge. Increasing roundness of a post increases the critical size value of that post for a given gap length.

In addition to critical size, posts of different shapes may also affect particle velocity given constant applied pressure. Given an applied pressure, arrays with triangular posts will result in a larger particle velocity than those with circular posts. Furthermore, the rate of particle velocity increase upon increasing pressure is also greater in triangular post arrays than circular post arrays.

Cartridges described herein may comprise a Seal/Lid on the top and/or bottom and a separation layer that comprises a plurality of obstacles that promote separation, a fluidic layer, and a void space or crumple zone that allows fabrication of the cartridge without deforming the plurality of obstacles. The plurality of obstacles may be arrayed in rows and columns, such that gaps configured to allow the passage of fluid and cells are formed. The obstacles may be arrayed such that they are stacked with no or minimal offset between repeating rows. Two or more cartridges may be stacked or connected in series or parallel to achieve greater separation or higher throughput.

As similar devices or microfluidic cartridges operate on a sub-millimeter scale and handles micro-liters, nano-liters, or smaller quantities of fluids, a major obstacle in manufacturing is avoiding damage or deformation of obstacles during embossing or assembly. For example, handling of the chip may result in pressure to the planar support, especially when planar supports are pressed together, which may then result in deformation or destruction of the planar support(s), obstacles (i.e. an array of obstacles), and the various separation lanes. Such deformation or destruction may result in a significant loss of performance in purifying particles or cells or may completely compromise the function of the microfluidic cartridge. In order to avoid potential deformations and defects during manufacturing and assembly, other microfluidic systems require slower manufacturing runs or accept diminished performance.

In an aspect, the present disclosure provides a microfluidic cartridge for purifying cells or particles. The microfluidic cartridge may include a first planar support. The first planar support may comprise a top surface and a bottom surface. The device may include a second planar support. The second planar support may comprise a top surface and a bottom surface. A top surface may comprise at least one embedded channel extending from one or more inlets to one or more outlets. The at least one embedded channel may comprise an array of obstacles. The bottom surface of the first and second planar support may comprise a void space. The void space may be configured to be deformed when the bottom of the first planar support is pressed to the bottom of the second planar support. Separation according to this description occurs along a channel embedded in a planar support, the channel comprising a plurality of obstacles. For cartridges of this description a first and a second planar surface may be utilized. The first and second planar surfaces may be stacked (e.g., bottom to bottom or top to bottom with a spacer doubling the throughput and separation capacity while maintaining a small footprint. A top surface of a first and/or second planar surface may comprise at least 1 embedded channel to about 500 embedded channels. A top surface may comprise at least 1 embedded channel to about 2 embedded channels, 1 embedded channel to about 5 embedded channels, 1 embedded channel to about 20 embedded channels, 1 embedded channel to about 50 embedded channels, 1 embedded channel to about 100 embedded channels, 1 embedded channel to about 500 embedded channels, about 2 embedded channels to about 5 embedded channels, about 2 embedded channels to about 20 embedded channels, about 2 embedded channels to about 50 embedded channels, about 2 embedded channels to about 100 embedded channels, about 2 embedded channels to about 500 embedded channels, about 5 embedded channels to about 20 embedded channels, about 5 embedded channels to about 50 embedded channels, about 5 embedded channels to about 100 embedded channels, about 5 embedded channels to about 500 embedded channels, about 20 embedded channels to about 50 embedded channels, about 20 embedded channels to about 100 embedded channels, about 20 embedded channels to about 500 embedded channels, about 50 embedded channels to about 100 embedded channels, about 50 embedded channels to about 500 embedded channels, or about 100 embedded channels to about 500 embedded channels. A top surface may comprise at least 1 embedded channel, about 2 embedded channels, about 5 embedded channels, about 20 embedded channels, about 50 embedded channels, about 100 embedded channels, or about 500 embedded channels. A top surface may comprise at least 1 embedded channel, about 2 embedded channels, about 5 embedded channels, about 20 embedded channels, about 50 embedded channels, or about 100 embedded channels. A top surface may comprise at least at most about 2 embedded channels, about 5 embedded channels, about 20 embedded channels, about 50 embedded channels, about 100 embedded channels, or about 500 embedded channels. A top surface or a first or second planar surface may comprise about 28 channels (56 when stacked). An additional third, fourth, fifth, or sixth planar surface may also comprise a similar amount of embedded channels as the first or second planar surface.

The microfluidic cartridge may comprise at least 1 inlet to about 50 inlets. The microfluidic cartridge may comprise at least 1 inlet to about 2 inlets, 1 inlet to about 5 inlets, 1 inlet to about 10 inlets, 1 inlet to about 20 inlets, 1 inlet to about 50 inlets, about 2 inlets to about 5 inlets, about 2 inlets to about 10 inlets, about 2 inlets to about 20 inlets, about 2 inlets to about 50 inlets, about 5 inlets to about 10 inlets, about 5 inlets to about 20 inlets, about 5 inlets to about 50 inlets, about 10 inlets to about 20 inlets, about 10 inlets to about 50 inlets, or about 20 inlets to about 50 inlets. The microfluidic cartridge may comprise at least 1 inlet, about 2 inlets, about 5 inlets, about 10 inlets, about 20 inlets, or about 50 inlets. The microfluidic cartridge may comprise at least 1 inlet, about 2 inlets, about 5 inlets, about 10 inlets, or about 20 inlets. The microfluidic cartridge may comprise at least at most about 2 inlets, about 5 inlets, about 10 inlets, about 20 inlets, or about 50 inlets. The inlets may be fed by a common fluidic system or a dual fluidic system (one for buffer/diluent and one for sample).

The microfluidic cartridge may comprise at least 1 outlet to about 50 outlets. The microfluidic cartridge may comprise at least 1 outlet to about 2 outlets, 1 outlet to about 5 outlets, 1 outlet to about 10 outlets, 1 outlet to about 20 outlets, 1 outlet to about 50 outlets, about 2 outlets to about 5 outlets, about 2 outlets to about 10 outlets, about 2 outlets to about 20 outlets, about 2 outlets to about 50 outlets, about 5 outlets to about 10 outlets, about 5 outlets to about 20 outlets, about 5 outlets to about 50 outlets, about 10 outlets to about 20 outlets, about 10 outlets to about 50 outlets, or about 20 outlets to about 50 outlets. The microfluidic cartridge may comprise at least 1 outlet, about 2 outlets, about 5 outlets, about 10 outlets, about 20 outlets, or about 50 outlets. The microfluidic cartridge may comprise at least 1 outlet, about 2 outlets, about 5 outlets, about 10 outlets, or about 20 outlets. The microfluidic cartridge may comprise at least at most about 2 outlets, about 5 outlets, about 10 outlets, about 20 outlets, or about 50 outlets. The outlets may feed a common fluidic system or a dual fluidic system (one for waste and one for enriched target cells or particles).

The cartridge comprising two or more planar surfaces may comprise a void space to protect the array of obstacles in the lanes as their small size leads their susceptibility to deformation, leading to malfunction.

The void space of the microfluidic cartridge may be configured to deform, bend, swell, collapse, or crumple. The void space may be configured to protect the obstacles, channels, inlets, outlets, planar surfaces, or any combination thereof, from damage, displacement, deformation, or malfunction. The void space may comprise a crumple zone that is configured to protect the obstacles, channels, inlets, outlets, planar surfaces, or any combination thereof, from damage, displacement, deformation, or malfunction. The void space may have a volume of about 1 cubic μm to about 10,000 cubic μm. The void space may have a volume of about 1 cubic μm to about 5 cubic μm, about 1 cubic μm to about 10 cubic μm, about 1 cubic μm to about 30 cubic μm, about 1 cubic μm to about 50 cubic μm, about 1 cubic μm to about 100 cubic μm, about 1 cubic μm to about 300 cubic μm, about 1 cubic μm to about 1,000 cubic μm, about 1 cubic μm to about 3,000 cubic μm, about 1 cubic μm to about 10,000 cubic μm, about 5 cubic μm to about 10 cubic μm, about 5 cubic μm to about 30 cubic μm, about 5 cubic μm to about 50 cubic μm, about 5 cubic μm to about 100 cubic μm, about 5 cubic μm to about 300 cubic μm, about 5 cubic μm to about 1,000 cubic μm, about 5 cubic μm to about 3,000 cubic μm, about 5 cubic μm to about 10,000 cubic μm, about 10 cubic μm to about 30 cubic μm, about 10 cubic μm to about 50 cubic μm, about 10 cubic μm to about 100 cubic μm, about 10 cubic μm to about 300 cubic μm, about 10 cubic μm to about 1,000 cubic μm, about 10 cubic μm to about 3,000 cubic μm, about 10 cubic μm to about 10,000 cubic μm, about 30 cubic μm to about 50 cubic μm, about 30 cubic μm to about 100 cubic μm, about 30 cubic μm to about 300 cubic μm, about 30 cubic μm to about 1,000 cubic μm, about 30 cubic u m to about 3,000 cubic μm, about 30 cubic μm to about 10,000 cubic μm, about 50 cubic μm to about 100 cubic μm, about 50 cubic μm to about 300 cubic μm, about 50 cubic μm to about 1,000 cubic μm, about 50 cubic μm to about 3,000 cubic μm, about 50 cubic μm to about 10,000 cubic μm, about 100 cubic μm to about 300 cubic μm, about 100 cubic μm to about 1,000 cubic μm, about 100 cubic μm to about 3,000 cubic μm, about 100 cubic μm to about 10,000 cubic μm, about 300 cubic μm to about 1,000 cubic μm, about 300 cubic μm to about 3,000 cubic μm, about 300 cubic μm to about 10,000 cubic μm, about 1,000 cubic μm to about 3,000 cubic μm, about 1,000 cubic μm to about 10,000 cubic μm, or about 3,000 cubic μm to about 10,000 cubic μm. The void space may have a volume of about 1 cubic μm, about 5 cubic μm, about 10 cubic μm, about 30 cubic μm, about 50 cubic μm, about 100 cubic μm, about 300 cubic μm, about 1,000 cubic μm, about 3,000 cubic μm, or about 10,000 cubic μm. The void space may have a volume of at least about 1 cubic μm, about 5 cubic μm, about 10 cubic μm, about 30 cubic μm, about 50 cubic μm, about 100 cubic μm, about 300 cubic μm, about 1,000 cubic μm, or about 3,000 cubic μm. The void space may have a volume of at most about 5 cubic μm, about 10 cubic μm, about 30 cubic μm, about 50 cubic μm, about 100 cubic μm, about 300 cubic μm, about 1,000 cubic μm, about 3,000 cubic μm, or about 10,000 cubic μm.

The bottom surface of a cartridge may comprise a plurality of void spaces shown here arranged into strips that run parallel with the length of the planar support. The void spaces may run beneath the array or column of obstacles or the lanes formed by the columns of obstacles fabricated on the top surface of the planar support. The top surface of the planar support may comprise a plurality of individual obstacles formed into arrays or columns creating gaps to allow the flow of fluid, cells, and/or particles. Beneath the obstacles embedded in the bottom surface of the planar support may be a void space. The area of the void space (length×width) opposite the lane can be at least about 80% of the area (length×width) of the lane. In certain embodiments, the area of the void space (length×width) opposite the lane can be at least about 90%, 100%, 110%, or 120% up to and including about 150% of the area (length×width) of the lane.

In one configuration the void spaces of the two planar supports may be symmetrical or nearly symmetrical and pressed back to back. However alternative arrangements are also possible, such as stacked with the void space above or below the obstacle layer.

The void space may be separated into two or more void spaces. The void space may be separated into at least 3, 4, 5, 6, 7, 8, 9, 10, 11, 12, 13, 14, 15, 16, 17, 18, 19, 20, 21, 22, 23, 24, 25, 26, 27, 28, 29, or 30 void spaces. The void space may be separated into exactly two void spaces. There may be a 1:1 ratio between channels or lanes and void spaces for each planar support comprising obstacles.

The planar support may be fabricated from two layers of material bonded together. The layers may be bonded together by adhesive, polymer, or thermoplastic. The layers may be comprised of polymer or thermoplastic. The polymer or thermoplastic layers or bonding material may be comprised of high-density polyethylene (HDPE), polypropylene (PP), polyethylene terephthalate (PT), polycarbonate (PC), or cyclic olefin copolymer (COC).

The top layer of a cartridge may comprise an array of obstacles in at least one embedded channel, void space, at least one inlet, at least one outlet, or combination thereof. The bottom layer of a cartridge may comprise an array of obstacles in at least one embedded channel, void space, at least one inlet, at least one outlet, or combination thereof. The layers may be positioned to where the planar supports are bonded together on their side surfaces, bottom surfaces, or top surfaces. The void space may be inside the interface of the planar supports bonded together, or outside the interface.

The microfluidic cartridge may further comprise an obstacle bonding layer that is bonded to the surface of the planar support and a top surface of the array of obstacles in the embedded channels to prevent fluid or sample from flowing over the array of obstacles during operation of the cartridge. The obstacle bonding layer may be metallic, polymer, or thermoplastic. The obstacle bonding layer may be a cover or a film. The polymer or thermoplastic layers or bonding material may be comprised of high-density polyethylene (HDPE), polypropylene (PP), polyethylene terephthalate (PT), polycarbonate (PC), or cyclic olefin copolymer (COC). The microfluidic cartridge may comprise two obstacle bonding layers on the outside of the top planar support. The microfluidic cartridge may comprise a single obstacle bonding layer in the middle of the cartridge as the bonding agent for the planar supports. The obstacle bonding layer may comprise one or more passages fluidically connected to the one or more inlets of the embedded channels which permit the flow of sample into the channels and one or more passages fluidically connected to the one or more outlets of the channels that permit the flow of fluid out from the one or more outlets. Such an obstacle layer may comprise at least about 1, at least about 2, at least about 3, at least about 4, at least about 5, at least about 10, at least about 20, at least about 30, at least about 50, or at least about 100 passages fluidically connected to the one or more inlets or one or more outlets of the embedded channels.

The microfluidic cartridge may have the obstacles positioned so as to define a critical size of the cartridge such that when a sample is applied to an inlet of the cartridge and flows to an outlet, particles or cells in the sample larger than the critical size are separated from particles or cells in the sample smaller than the critical size. Each obstacle may have its own individual sub-critical size, the sum the individual obstacles defining the critical size of the cartridge. The one or more outlets of the cartridge may comprise at least one product outlet, wherein target particles or cells, having a size larger than the critical size of the cartridge, are directed to the at least one product outlet. The one or more outlets of the cartridge may comprise at least one product outlet, wherein target particles or cells, having a size larger than the critical size of the cartridge, are directed to the at least one product outlet. The cartridge may have at least about 1, at least about 2, at least about 3, at least about 5, at least about 10, or at least about 50 product outlets. The particles, or cells, having a size larger than the critical size, may flow to the at least one product outlet. The cartridge may have at least about 1, at least about 2, at least about 3, at least about 5, at least about 10, or at least about 50 waste outlets.

The obstacles used in the cartridge may take the shape of columns or be triangular, square, rectangular, diamond shaped, trapezoidal, hexagonal, teardrop shaped, circular shape, semicircular shape, triangular with top side horizontal shape, and triangular with bottom side horizontal shape. In addition, adjacent obstacles may have a geometry such that the portions of the obstacles defining the gap are either symmetrical or asymmetrical about the axis of the gap that extends in the direction of bulk fluid flow. The obstacles may have vertices that extend into parallel gaps such that the gaps are flanked on either side by one or more vertices pointing toward one another but not directly opposite one another. The obstacles may have vertices that extend into perpendicular gaps such that the gaps are flanked on either side by vertices pointing toward one another and that are directly opposite one another. Obstacle location and shape can vary in a single chip. Additional obstacles can be added to any location of the device for any specific requirement. Also, the shape of the obstacle can be different in a device. Any combinations of posts shape, size and location can be used for specific requirement. The cartridge may be comprised of only diamond or hexagonal shaped obstacles.

The obstacle shapes may be elongated perpendicularly to the direction of fluid flow such that they have a horizontal length (P1) that is different from their vertical length (P2). P1 may have a length of about 1 μm to about 160 μm. P1 may have a length of about 1 μm to about 10 μm, about 1 μm to about 15 μm, about 1 μm to about 30 μm, about 1 μm to about 40 μm, about 1 μm to about 80 μm, about 1 μm to about 160 μm, about 10 μm to about 15 μm, about 10 μm to about 30 μm, about 10 μm to about 40 μm, about 10 μm to about 80 μm, about 10 μm to about 160 μm, about 15 μm to about 30 μm, about 15 μm to about 40 μm, about 15 μm to about 80 μm, about 15 μm to about 160 μm, about 30 μm to about 40 μm, about 30 μm to about 80 μm, about 30 μm to about 160 μm, about 40 μm to about 80 μm, about 40 μm to about 160 μm, or about 80 μm to about 160 μm. P1 may have a length of about 1 μm, about 10 μm, about 15 μm, about 30 μm, about 40 μm, about 80 μm, or about 160 μm. P1 may have a length of at least about 1 μm, about 10 μm, about 15 μm, about 30 μm, about 40 μm, or about 80 μm. P1 may have a length of at most about 10 μm, about 15 μm, about 30 μm, about 40 μm, about 80 μm, or about 160 μm. P2 may have a length of about 1 μm to about 160 μm. P2 may have a length of about 1 μm to about 10 μm, about 1 μm to about 15 μm, about 1 μm to about 30 μm, about 1 μm to about 40 μm, about 1 μm to about 80 μm, about 1 μm to about 160 μm, about 10 μm to about 15 μm, about 10 μm to about 30 μm, about 10 μm to about 40 μm, about 10 μm to about 80 μm, about 10 μm to about 160 μm, about 15 μm to about 30 μm, about 15 μm to about 40 μm, about 15 μm to about 80 μm, about 15 μm to about 160 μm, about 30 μm to about 40 μm, about 30 μm to about 80 μm, about 30 μm to about 160 μm, about 40 μm to about 80 μm, about 40 μm to about 160 μm, or about 80 μm to about 160 μm. P2 may have a length of about 1 μm, about 10 μm, about 15 μm, about 30 μm, about 40 μm, about 80 μm, or about 160 μm. P2 may have a length of at least about 1 μm, about 10 μm, about 15 μm, about 30 μm, about 40 μm, or about 80 μm. P2 may have a length of at most about 10 μm, about 15 μm, about 30 μm, about 40 μm, about 80 μm, or about 160 μm. P1 may be longer than P2 by about 25% to about 200%. P1 may be longer than P2 by about 25% to about 50%, about 25% to about 75%, about 25% to about 100%, about 25% to about 150%, about 25% to about 200%, about 50% to about 75%, about 50% to about 100%, about 50% to about 150%, about 50% to about 200%, about 75% to about 100%, about 75% to about 150%, about 75% to about 200%, about 100% to about 150%, about 100% to about 200%, or about 150% to about 200%. P1 may be longer than P2 by about 25%, about 50%, about 75%, about 100%, about 150%, or about 200%. P1 may be longer than P2 by at least about 25%, about 50%, about 75%, about 100%, or about 150%. P1 may be longer than P2 by at most about 50%, about 75%, about 100%, about 150%, or about 200%.

The microfluidic cartridge may comprise obstacles as an array of obstacles. The obstacles may be arranged in in columns and in rows that form discreet arrays. The array of obstacles may compromise at least about 5 columns to about 50 columns. The array of obstacles may compromise at least about 5 columns to about 10 columns, about 5 columns to about 28 columns, about 5 columns to about 29 columns, about 5 columns to about 30 columns, about 5 columns to about 50 columns, about 10 columns to about 28 columns, about 10 columns to about 29 columns, about 10 columns to about 30 columns, about 10 columns to about 50 columns, about 28 columns to about 29 columns, about 28 columns to about 30 columns, about 28 columns to about 50 columns, about 29 columns to about 30 columns, about 29 columns to about 50 columns, or about 30 columns to about 50 columns. The array of obstacles may compromise at least about 5 columns, about 10 columns, about 28 columns, about 29 columns, about 30 columns, or about 50 columns. The array of obstacles may compromise at least about 5 columns, about 10 columns, about 28 columns, about 29 columns, or about 30 columns. The array of obstacles may compromise at least at most about 10 columns, about 28 columns, about 29 columns, about 30 columns, or about 50 columns. The array of obstacles may compromise at least about 20 rows to about 500 rows. The array of obstacles may compromise at least about 20 rows to about 30 rows, about 20 rows to about 60 rows, about 20 rows to about 100 rows, about 20 rows to about 200 rows, about 20 rows to about 500 rows, about 30 rows to about 60 rows, about 30 rows to about 100 rows, about 30 rows to about 200 rows, about 30 rows to about 500 rows, about 60 rows to about 100 rows, about 60 rows to about 200 rows, about 60 rows to about 500 rows, about 100 rows to about 200 rows, about 100 rows to about 500 rows, or about 200 rows to about 500 rows. The array of obstacles may compromise at least about 20 rows, about 30 rows, about 60 rows, about 100 rows, about 200 rows, or about 500 rows. The array of obstacles may compromise at least about 20 rows, about 30 rows, about 60 rows, about 100 rows, or about 200 rows. The array of obstacles may compromise at least at most about 30 rows, about 60 rows, about 100 rows, about 200 rows, or about 500 rows. Multiple arrays of obstacles can be arranged in discrete lanes. The array of obstacles of the first or second planar support forms about 10 lanes to about 50 lanes. The array of obstacles of the first or second planar support forms about 10 lanes to about 20 lanes, about 10 lanes to about 28 lanes, about 10 lanes to about 30 lanes, about 10 lanes to about 50 lanes, about 20 lanes to about 28 lanes, about 20 lanes to about 30 lanes, about 20 lanes to about 50 lanes, about 28 lanes to about 30 lanes, about 28 lanes to about 50 lanes, or about 30 lanes to about 50 lanes. The array of obstacles of the first or second planar support forms about 10 lanes, about 20 lanes, about 28 lanes, about 30 lanes, or about 50 lanes. The array of obstacles of the first or second planar support forms at least about 10 lanes, about 20 lanes, about 28 lanes, or about 30 lanes. The array of obstacles of the first or second planar support forms at most about 20 lanes, about 28 lanes, about 30 lanes, or about 50 lanes.

Each cartridge may comprise at least one, at least two, at least three, or at least four sets of arrays of obstacles. Each planar top surface may comprise at least one or at least two arrays. The cartridge may comprise a total of about 20 lanes to about 100 lanes. The cartridge may comprise a total of about 20 lanes to about 40 lanes, about 20 lanes to about 56 lanes, about 20 lanes to about 60 lanes, about 20 lanes to about 100 lanes, about 40 lanes to about 56 lanes, about 40 lanes to about 60 lanes, about 40 lanes to about 100 lanes, about 56 lanes to about 60 lanes, about 56 lanes to about 100 lanes, or about 60 lanes to about 100 lanes. The cartridge may comprise a total of about 20 lanes, about 40 lanes, about 56 lanes, about 60 lanes, or about 100 lanes. The cartridge may comprise a total of at least about 20 lanes, about 40 lanes, about 56 lanes, or about 60 lanes. The cartridge may comprise a total of at most about 40 lanes, about 56 lanes, about 60 lanes, or about 100 lanes.

The inlets, outlets, or both, of the microfluidic cartridge may be in fluid connection with pumps or motors to drive the flow of fluids within and outside of the cartridge. The inlets, outlets, or both, may be fluidically connected to at least about 1, 2, 3, 4, 5, 6, 7, 8, 9, or 10 pumps. The pumps may be peristaltic pumps. The pumps may be fluidically connected to each other or isolated. The inlets and outlets of the cartridge may be in fluidic connection with two peristaltic pumps connected in parallel to each other. The inlets and outlets of the cartridge may be in fluidic connection with two peristaltic pumps connected in serial to each other.

The microfluidic cartridge may be fabricated from a metal, polymer, or thermoplastic. The polymer or thermoplastic may be comprised of high-density polyethylene (HDPE), polypropylene (PP), polyethylene terephthalate (PT), polycarbonate (PC), or cyclic olefin copolymer (COC). In an example, the microfluidic cartridge is comprised of cyclic olefin copolymer.

The present disclosure also provides for a microfluid assembly comprising a plurality of microfluidic cartridges in fluidic connection. The cartridges in the assembly may be stacked or layered. The plurality of microfluidic cartridges may comprise at least about 2, 3, 4, 5, 6, 7, 8, 9, 10, 20, or 30 cartridges. The plurality of cartridges may be fluidically connected in serial or in parallel.

During DLD, a fluid sample containing cells is introduced into a device at an inlet and is carried along with fluid flowing through the device to outlets. As cells in the sample traverse the device, they encounter posts or other obstacles that have been positioned to form gaps or pores through which the cells must pass. Each successive row of obstacles is displaced relative to the preceding row so as to form an array direction that differs from the direction of fluid flow in the flow channel. The “tilt angle” defined by these two directions, together with the width of gaps between obstacles, the shape of obstacles, and the orientation of obstacles forming gaps are primary factors in determining a “critical size” for an array. Cells having a size greater than the critical size travel in the array direction, rather than in the direction of bulk fluid flow and particles having a size less than the critical size travel in the direction of bulk fluid flow. In devices used for leukapheresis-derived compositions, array characteristics may be chosen that result in white blood cells being diverted in the array direction whereas red blood cells and platelets continue in the direction of bulk fluid flow. In order to separate a chosen type of leukocyte from others having a similar size, a carrier may then be used that binds to that cell in a way that promotes DLD separation and which thereby results in a complex that is larger than uncomplexed leukocytes. It may then be possible to carry out a separation on a device having a critical size smaller than the complexes but bigger than the uncomplexed cells.

A device can be made using any of the materials from which micro- and nano-scale fluid handling devices are typically fabricated, including silicon, glasses, plastics, and hybrid materials. A diverse range of thermoplastic materials suitable for microfluidic fabrication is available, offering a wide selection of mechanical and chemical properties that can be leveraged and further tailored for specific applications. In an aspect, the microfluidic cartridge may be fabricated by soft embossing and UV-light curing.

The microfluidic cartridge (or device, cassette, chip, etc.) may be made by techniques including Replica molding, Soft lithography with PDMS, Thermoset polyester, Embossing, soft embossing, hot embossing, Roll to Roll embossing, Injection Molding, Laser Ablation, UV-light curing, and combinations thereof. Further details can be found in “Disposable microfluidic devices: fabrication, function and application” by Fiorini, et al. (BioTechniques 38:429-446 (March 2005)), which is hereby incorporated by reference herein in its entirety. The book “Lab on a Chip Technology” edited by Keith E. Herold and Avraham Rasooly, Caister Academic Press Norfolk UK (2009) is another resource for methods of fabrication and is hereby incorporated by reference herein in its entirety.

High-throughput embossing methods such as reel-to-reel processing of thermoplastics is an attractive method for industrial microfluidic chip production. The use of single chip hot embossing can be a cost-effective technique for realizing high-quality microfluidic devices during the prototyping stage. Methods for the replication of microscale features in two thermoplastics, polymethylmethacrylate (PMMA) and/or polycarbonate (PC), are described in “Microfluidic device fabrication by thermoplastic hot-embossing” by Yang, et al. (Methods Mol. Biol. 949:115-23 (2013)), which is hereby incorporated by reference herein in its entirety

The flow channel can be constructed using two or more pieces which, when assembled, form a closed cavity (preferably one having orifices for adding or withdrawing fluids) having the obstacles disposed within it. The obstacles can be fabricated on one or more pieces that are assembled to form the flow channel, or they can be fabricated in the form of an insert that is sandwiched between two or more pieces that define the boundaries of the flow channel.

The obstacles may be solid bodies that extend in an array laterally across the flow channel and longitudinally along the channel from the inlets to the outlets. Where an obstacle is integral with (or an extension of) one of the faces of the flow channel at one end of the obstacle, the other end of the obstacle can be sealed to or pressed against the opposite face of the flow channel. A small space (preferably too small to accommodate any particles of interest for an intended use) is tolerable between one end of an obstacle and a face of the flow channel, provided the space does not adversely affect the structural stability of the obstacle or the relevant flow properties of the device.

Surfaces can be coated to modify their properties and polymeric materials employed to fabricate devices, can be modified in many ways. In some cases, functional groups such as amines or carboxylic acids that are either in the native polymer or added by means of wet chemistry or plasma treatment are used to crosslink proteins or other molecules. DNA can be attached to COC and PMMA substrates using surface amine groups. Surfactants such as Pluronic® can be used to make surfaces hydrophilic and protein repellant by adding Pluronic® to PDMS formulations. In some cases, a layer of PMMA is spin coated on a device, e.g., microfluidic chip and PMMA is “doped” with hydroxypropyl cellulose to vary its contact angle.

To reduce non-specific adsorption of cells or compounds, e.g., released by lysed cells or found in biological samples, onto the channel walls, one or more walls may be chemically modified to be non-adherent or repulsive. The walls may be coated with a thin film coating (e.g., a monolayer) of commercial non-stick reagents, such as those used to form hydrogels. Additional examples of chemical species that may be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers may also be employed to repel oppositely charged species. The type of chemical species used for repulsion and the method of attachment to the channel walls can depend on the nature of the species being repelled and the nature of the walls and the species being attached. Such surface modification techniques are well known in the art. The walls may be functionalized before or after the device is assembled.

IV. Separation Processes that Use DLD

The DLD devices described herein can be used to purify cells, cellular fragments, cell adducts, or nucleic acids. Separation and purification of blood components using devices can be found, for example, in US Publication No. US 2016/0139012, the teaching of which is incorporated by reference herein in its entirety.

The purity, yields and viability of cells produced by DLD methods will vary based on a number of factors including the nature of the starting material, the exact procedure employed and the characteristics of the DLD device. Preferably, purifications, yields and viabilities of at least 60% should be obtained with, higher percentages, at least 70, 80 or 90% being more preferred.

In an aspect, the present disclosure provides methods for enriching target particles or target cells of a predetermined size from contaminants in a sample. Methods for enriching target particles or target cells use any cartridge, microfluidic cartridge, cassette, chip, device, fluidic device, or microfluidic device as described elsewhere herein. A method may comprise obtaining a sample comprising target particles or target cells and the contaminants. The method may further comprise separating the target particles or target cells from the contaminants by applying the sample to one or more sample inlets on any of the cartridges, cassettes, or devices described herein. The method may further comprise flowing the sample to the outlets on any of the cartridges, cassettes, or devices described herein. The method may further comprise obtaining a product enriched in target particles or target cells from one or more outlets while removing the contaminants. The method may result in a superior ability to purify or separate cells or particles from contaminants, creating greater cells yields, improved ability to expand the product in vitro, and an enriched cell product more amenable to transduction or other genetic engineering.

The method may entail the used of deterministic lateral displacement whereby the device has a critical size as described herein and the contaminants and the target particles or target cells are separated on the basis of having different critical size. The method may comprise flowing a sample containing the target particles or target cells and contaminants to any of the of the cartridges, cassettes, or devices described herein, wherein the target particles or target cells have a size larger than a critical size of the array of obstacles and at least some contaminants have sizes smaller than the critical size of the array of obstacles and wherein target cells or target particles flow to the one or more product outlets where a product enriched in target cells or target particles is obtained and contaminants with a size smaller than the critical size of the array of obstacles flow to one more waste outlets. The method may comprise flowing a sample containing the target particles or target cells and contaminants to any of the of the cartridges, cassettes, or devices described herein, wherein the target particles or target cells have a size smaller than a critical size of the array of obstacles and at least some contaminants have sizes larger than the critical size of the array of obstacles and wherein target cells or target particles flow to the one or more product outlets where a product enriched in target cells or target particles is obtained and contaminants with a size larger than the critical size of the array of obstacles flow to one more waste outlets.

The method may comprise flowing a sample containing the target particles or target cells and contaminants to any of the of the cartridges, cassettes, or devices described herein, at a constant flow rate or a variable flow rate. The cartridge flow rate of the method may be about 400 mL per hour. The cartridge flow rate of the method may be about 100 mL per hour to about 1,000 mL per hour. The cartridge flow rate of the method may be about 100 mL per hour to about 200 mL per hour, about 100 mL per hour to about 400 mL per hour, about 100 mL per hour to about 800 mL per hour, about 100 mL per hour to about 1,000 mL per hour, about 200 mL per hour to about 400 mL per hour, about 200 mL per hour to about 800 mL per hour, about 200 mL per hour to about 1,000 mL per hour, about 400 mL per hour to about 800 mL per hour, about 400 mL per hour to about 1,000 mL per hour, or about 800 mL per hour to about 1,000 mL per hour. The cartridge flow rate of the method may be about 100 mL per hour, about 200 mL per hour, about 400 mL per hour, about 800 mL per hour, or about 1,000 mL per hour. The cartridge flow rate of the method may be at least about 100 mL per hour, about 200 mL per hour, about 400 mL per hour, or about 800 mL per hour. The cartridge flow rate of the method may be at most about 200 mL per hour, about 400 mL per hour, about 800 mL per hour, or about 1,000 mL per hour.

The method may comprise an internal pressure within the cartridge. The internal pressure of the cartridge may be at least about 15 pounds per square inch. The internal pressure of the cartridge may be at least about 1.5 pounds per square inch to about 50 pounds per square inch. The internal pressure of the cartridge may be at least about 1.5 pounds per square inch to about 5 pounds per square inch, about 1.5 pounds per square inch to about 10 pounds per square inch, about 1.5 pounds per square inch to about 15 pounds per square inch, about 1.5 pounds per square inch to about 20 pounds per square inch, about 1.5 pounds per square inch to about 50 pounds per square inch, about 5 pounds per square inch to about 10 pounds per square inch, about 5 pounds per square inch to about 15 pounds per square inch, about 5 pounds per square inch to about 20 pounds per square inch, about 5 pounds per square inch to about 50 pounds per square inch, about 10 pounds per square inch to about 15 pounds per square inch, about 10 pounds per square inch to about 20 pounds per square inch, about 10 pounds per square inch to about 50 pounds per square inch, about 15 pounds per square inch to about 20 pounds per square inch, about 15 pounds per square inch to about 50 pounds per square inch, or about 20 pounds per square inch to about 50 pounds per square inch. The internal pressure of the cartridge may be at least about 1.5 pounds per square inch, about 5 pounds per square inch, about 10 pounds per square inch, about 15 pounds per square inch, about 20 pounds per square inch, or about 50 pounds per square inch. The internal pressure of the cartridge may be at least about 1.5 pounds per square inch, about 5 pounds per square inch, about 10 pounds per square inch, about 15 pounds per square inch, or about 20 pounds per square inch. The internal pressure of the cartridge may be at least at most about 5 pounds per square inch, about 10 pounds per square inch, about 15 pounds per square inch, about 20 pounds per square inch, or about 50 pounds per square inch. Separation of cells in a sample can be performed by positive or negative selection of cell types using DLD and be collected in an output tube. Accordingly, DLD can be used for generating a reduced platelet blood related sample. In certain embodiments, the reduced platelet blood related sample comprises a ratio of platelets to target cells of less than about 500:1. In certain embodiments, the reduced platelet blood related sample comprises a ratio of platelets to target cells of less than about 100:1. In certain embodiments, the reduced platelet blood related sample comprises a ratio of platelets to target cells of less than about 10:1. In certain embodiments, the reduced platelet blood related sample comprises a ratio of platelets to target cells of less than about 5:1. In certain embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1.

Accordingly, DLD can be used for generating a population of enriched target cells from a sample. In certain embodiments, the population of enriched target cells comprises a ratio of platelets to target cells of less than about 500:1. In certain embodiments, the population of enriched target cells comprises a ratio of platelets to target cells of less than about 100:1. In certain embodiments, the population of enriched target cells comprises a ratio of platelets to target cells of less than about 10:1. In certain embodiments, the population of enriched target cells comprises a ratio of platelets to target cells of less than about 5:1. In certain embodiments, the population of enriched target cells comprises a ratio of red blood cells to target cells of greater than about 100:1. In certain embodiments, the population of enriched target cells comprises a ratio of red blood cells to target cells of greater than about 250:1. In certain embodiments, the population of enriched target cells comprises a ratio of red blood cells to target cells of greater than about 500:1. In certain embodiments, the population of enriched target cells comprises a ratio of red blood cells to target cells of no greater than about 1,000:1. In some embodiments, density gradient separation is used for the isolation of lymphocytes. In some embodiments, density gradient separation is used for the isolation of hematopoietic stem cells. In some embodiments, density gradient separation is used for the isolation of mesenchymal stem cells. In certain embodiments, the isolation of peripheral blood mononuclear cells (PBMCs) is used for the isolation of T cells for the generation of chimeric antigen receptor T cells (CAR-T cells).

In certain embodiments, the enriched target cells comprise PBMCS and exhibit depletion of greater than about 75%, 80%, 85%, 90%, 95%, 97%, 98%, 99% of red blood cells from a starting sample. In certain embodiments, the enriched target cells comprise PBMCS and exhibit depletion of greater than about 75%, 80%, 85%, 90%, 95%, 97%, 98%, 99% of platelet cells from a starting sample. In certain embodiments, the enriched target cells comprise PBMCS and exhibit depletion of greater than about 75%, 80%, 85%, 90%, 95%, 97%, 98%, 99% of red blood cells and platelet cells from a starting sample.

### Dielectrophoresis

Methods comprising dielectrophoresis (DEP) for separating different cell types can be useful for obtaining one or more target cells from a blood related sample. Dielectrophoresis (DEP) is a phenomenon in which particles, or cells, exposed to the gradient of an electric field are polarized depending on the characteristics of the cells and the medium that surrounds them. See U.S. Pat. No. 10,078,066; See also Douglas T A et al. “Separation of Macrophages and Fibroblasts Using Contactless Dielectrophoresis and a Novel ImageJ Macro.” Bioelectricity. 2019; 1 (1): 49-55. doi: 10.1089/bioe.2018.0004. Such polarization induces movement of the cells along the gradient of the electric field. Accordingly, dielectrophoresis (DEP) can be used to trap cells or divert them from normal streamlines. For example, dielectrophoresis (DEP) can be used to positively or negatively select target cell from a population of cells. Contactless dielectrophoresis (DEP), which employs a polydimethylsiloxane (PDMS) microfluidic device containing a cell flow chamber can be used to facilitate dielectrophoresis (DEP) isolation of cell types. A polydimethylsiloxane (PDMS) microfluidic device generally comprises a chamber containing an array of 20 mircometer (μm) posts where cells trap based on the gradient of an applied electric field. The device also generally comprises contactless fluidic electrodes that are filled with conductive fluid and separated from the main channel by a thin polydimethylsiloxane (PDMS) membrane. Applying voltage using contactless electrodes filled with a concentrated buffer (e.g. 10× concentrated phosphate-buffered saline (PBS)) eliminates problems with cell mortality as is seen in traditional dielectrophoresis by preventing electrolysis and bubble formation in the microfluidic device, as well as avoiding contact between regions of high electric field and cells.

In addition to improving cellular viability, utilizing small post structures allows better control of cell selectivity by preventing pearl chaining and cell-cell interactions. Cells with different bioelectrical phenotypes are trapped in the main channel at different applied electric field frequencies. By modulating the applied frequency, the device can selectively trap some cells while allowing others to pass through the device. This selectivity allows separation of highly similar cell types in a label-free manner while maintaining high cellular viability such that they can be cultured or further characterized downstream. This method provides more selective and higher viability separation of cells, which allows more closely related and physically similar cells to be separated, while allowing less similar cells to be separated at a much higher efficiency.

Batch separation can be performed by trapping some of the cells while allowing other cells to flow through and be collected in an output tube. After turning off the voltage, trapped cells can be released from their posts and can be collected in another output tube. Accordingly, dielectrophoretic methods can be used for generating a population of enriched target cells from a sample. In certain embodiments, the population of enriched target cells comprises a ratio of platelets to target cells of less than about 500:1. In certain embodiments, the population of enriched target cells comprises a ratio of platelets to target cells of less than about 100:1. In certain embodiments, the population of enriched target cells comprises a ratio of platelets to target cells of less than about 10:1. In certain embodiments, the population of enriched target cells comprises a ratio of platelets to target cells of less than about 5:1. In certain embodiments, the population of enriched target cells comprises a ratio of red blood cells to target cells of greater than about 100:1. In certain embodiments, the population of enriched target cells comprises a ratio of red blood cells to target cells of greater than about 250:1. In certain embodiments, the population of enriched target cells comprises a ratio of red blood cells to target cells of greater than about 500:1. In certain embodiments, the population of enriched target cells comprises a ratio of red blood cells to target cells of no greater than about 1,000:1. In some embodiments, density gradient separation is used for the isolation of lymphocytes. In some embodiments, density gradient separation is used for the isolation of hematopoietic stem cells. In some embodiments, density gradient separation is used for the isolation of mesenchymal stem cells. In certain embodiments, the isolation of peripheral blood mononuclear cells (PBMCs) is used for the isolation of T cells for the generation of chimeric antigen receptor T cells (CAR-T cells).

### Acoustophoretic Isolation

Methods comprising acoustophoresis for separating different cell types can be useful for obtaining one or more target cells from a blood related sample. Acoustophoresis is a phenomenon in which cells, exposed to an acoustic pressure field, are separated based on the characteristics of the cells. See U.S. Pat. No. 10,640,760; See also Dutra, Brian et al. “A Novel Macroscale Acoustic Device for Blood Filtration.” Journal of medical devices vol. 12, 1 (2018): 0110081-110087. doi: 10.1115/1.4038498. The underlying principle of the acoustic separation is based on the nonuniform acoustic pressure field in the fluid established by an acoustic standing wave. The introduction of a particle in this acoustic pressure field leads to a scattering of the acoustic pressure. The acoustic pressure acting on the surface of the particle then consists of the sum of the incident acoustic standing wave and the scattered wave. The net time averaged force on the particle is determined by integrating the acoustic pressure on the surface of the particle (i.e. acoustic radiation force). In addition to the axial acoustic radiation force component, a three-dimensional acoustic wave also exerts lateral forces on the suspended particle, orthogonal to the axis. An axial component of the acoustic radiation force component directs particles to collect in planes at the pressure nodes or antinodes every half wavelength, determined by a positive or negative acoustic contrast factor, respectively. A lateral component of the acoustic radiation force component collects the cells within the planes to local clusters, where the cells grow in collective size until they reach critical mass and the gravity/buoyancy force causes the cells to sink or rise out of suspension, thus separating the cells. Separation of cells in a sample can be performed by positive or negative selection of cell types using acoustophoresis and be collected in an output tube. Accordingly, acoustophoretic isolation can be used for generating a population of enriched target cells from a sample. In certain embodiments, the population of enriched target cells comprises a ratio of platelets to target cells of less than about 500:1. In certain embodiments, the population of enriched target cells comprises a ratio of platelets to target cells of less than about 100:1. In certain embodiments, the population of enriched target cells comprises a ratio of platelets to target cells of less than about 10:1. In certain embodiments, the population of enriched target cells comprises a ratio of platelets to target cells of less than about 5:1. In certain embodiments, the population of enriched target cells comprises a ratio of red blood cells to target cells of greater than about 100:1. In certain embodiments, the population of enriched target cells comprises a ratio of red blood cells to target cells of greater than about 250:1. In certain embodiments, the population of enriched target cells comprises a ratio of red blood cells to target cells of greater than about 500:1. In certain embodiments, the population of enriched target cells comprises a ratio of red blood cells to target cells of no greater than about 1,000:1. In some embodiments, density gradient separation is used for the isolation of lymphocytes. In some embodiments, density gradient separation is used for the isolation of hematopoietic stem cells. In some embodiments, density gradient separation is used for the isolation of mesenchymal stem cells. In certain embodiments, the isolation of peripheral blood mononuclear cells (PBMCs) is used for the isolation of T cells for the generation of chimeric antigen receptor T cells (CAR-T cells).

### Affinity Separation

Various techniques are known for separating components of a sample or biological material that make use of affinity-based separation techniques. Immunoaffinity methods may include selective labeling of certain components of a sample (e.g., antibody labeling) and separation of labeled and unlabeled components. To isolate cells from a biological sample, either pre-enriched or not, immunoaffinity capture utilizing an affinity molecule (e.g. an antibody, binding protein, aptamer, etc.) is used. Accordingly, immunoaffinity capture is used herein to refer to the use of affinity molecules (e.g. an antibody, binding protein, aptamer, etc.) to capture or isolate cells from a sample. Affinity molecules (e.g. an antibody, binding protein, aptamer, etc.) that bind specific cell marker proteins function as ligands to target cells, thereby providing a means to capture cells (either directly or indirectly) and permit their isolation from the sample. Examples of immunoaffinity capture techniques include, but are not limited to, immunoprecipitation, column affinity chromatography, magnetic-activated cell sorting, fluorescence-activated cell sorting, adhesion-based sorting and microfluidic-based sorting, either directly or using carriers. Affinity molecules (e.g. an antibody, binding protein, aptamer, etc.) in a homogeneous or a heterogenous cocktail may be utilized together, in a single solution, or may be utilized in two or more solutions that are used simultaneously or consecutively.

Magnetic separation methods typically include passing the sample through a separation column or incubation with a bead-based solution. Magnetic separation is a procedure for selectively retaining magnetic materials in a chamber or column disposed in a magnetic field. A target substance, including biological materials, may be magnetically labeled by attachment to a magnetic particle by means of a specific binding partner, which is conjugated to the particle. A suspension of the labeled target substance is then applied to the chamber. The target substance is retained in the chamber in the presence of a magnetic field. The retained target substance can then be eluted by changing the strength of, or by eliminating, the magnetic field. A matrix of material of suitable magnetic susceptibility may be placed in the chamber, such that when a magnetic field is applied to the chamber a high magnetic field gradient is locally induced close to the surface of the matrix. This permits the retention of weakly magnetized particles and the approach is referred to as high gradient magnetic separation (HGMS).

Accordingly, magnetic separation can be used for generating a population of enriched target cells from a sample. In certain embodiments, the population of enriched target cells comprises a ratio of platelets to target cells of less than about 500:1. In certain embodiments, the population of enriched target cells comprises a ratio of platelets to target cells of less than about 100:1. In certain embodiments, the population of enriched target cells comprises a ratio of platelets to target cells of less than about 10:1. In certain embodiments, the population of enriched target cells comprises a ratio of platelets to target cells of less than about 5:1. In certain embodiments, the population of enriched target cells comprises a ratio of red blood cells to target cells of greater than about 100:1. In certain embodiments, the population of enriched target cells comprises a ratio of red blood cells to target cells of greater than about 250:1. In certain embodiments, the population of enriched target cells comprises a ratio of red blood cells to target cells of greater than about 500:1. In certain embodiments, the population of enriched target cells comprises a ratio of red blood cells to target cells of no greater than about 1,000:1. In some embodiments, density gradient separation is used for the isolation of lymphocytes. In some embodiments, density gradient separation is used for the isolation of hematopoietic stem cells. In some embodiments, density gradient separation is used for the isolation of mesenchymal stem cells. In certain embodiments, the isolation of peripheral blood mononuclear cells (PBMCs) is used for the isolation of T cells for the generation of chimeric antigen receptor T cells (CAR-T cells).

Affinity molecules (e.g. an antibody, binding protein, aptamer, etc.) that bind biomarkers on the surface of platelets are thus useful. Known platelet surface biomarkers include, but are not limited to, CD36, CD41 (GP IIb/Illa), CD42a (GPIX), CD42b (GPIb), and CD61 (avb3, vitronectin receptor). Known platelet activation biomarkers appear on the platelet surface during activation and can be targeted. Platelet activation biomarkers include, but are not limited to, PAC-1 (activated IIb/IIIa), CD62P (P-selectin), CD31 (PECAM) and CD63. Red blood cell surface biomarkers can be useful for the targeting of affinity molecules (e.g. an antibody, binding protein, aptamer, etc.). Known red blood cell biomarkers include, but are not limited to, surface antigen A, surface antigen B, Rh factor, and CD235a.

In certain embodiments, the enriched target cell populations are not enriched by affinity-based separation. In certain embodiments, the enriched target cell populations are not enriched by magnetic-based separation.

The methods described herein result in enriched target cell populations that have reduced amounts of platelets and/or increased amounts or certain amounts of red blood cells. In some embodiments, the enriched target cells comprises less than 10%, 5%, 2%, or 1% platelets. In some embodiments, red blood cells are maintained at a level of 1×10⁴, 5×10⁴, 1×10⁵, 5×10⁵, 1×10⁶, 5×10⁶ red blood cells per microliter (μL). Additionally, red blood cells can be added to a collected cell target product from acoustophoresis. In some embodiments, the sample is a blood sample. In some embodiments, acoustophoresis is used for the isolation of peripheral blood mononuclear cells (PBMCs). In certain embodiments, the isolation of peripheral blood mononuclear cells (PBMCs) is used for the isolation of T cells for the generation of chimeric antigen receptor T cells (CAR-T cells). In some embodiments, acoustophoresis is used for the isolation of lymphocytes. In some embodiments, acoustophoresis is used for the isolation of hematopoietic stem cells. In some embodiments, acoustophoresis is used for the isolation of mesenchymal stem cells.

### Target Cells

The methods described herein allow for the enrichment, isolation, or purification of certain target cell and subsets, so that the target cells may be subsequently contacted by an activating agent and transduced with a viral vector comprising a polynucleotide. The target cells may be therapeutically relevant target cells. The target cells isolated may then be subjected to one or more steps comprising contacting the target cells with a nucleic acid or a virus comprising a nucleic acid.

Target cells comprise a type of cell, cell population, or composition of cells which are the desired cells to be enriched collected, isolated, or separated by the present invention. Generally, as disclosed herein, target cells can be any cell intended for immediate or downstream therapeutic use. The target cells disclosed herein are eukaryotic cells and generally consist of immune cells. Immune cells comprise cells originating from myeloid or lymphocyte lineages. In some embodiments, the therapeutic cell is a leukocyte. In some embodiments, the therapeutic cell is a lymphocyte. Lymphocytes cells can be identified by positivity for the cell surface marker CD45 (lymphocyte common antigen). In certain embodiments, the lymphocyte comprises natural killer cells, T cells, and/or B cells. In certain embodiments, the target cell is a T cell (e.g., CD3+). In certain embodiments, the target cell is a natural killer cell (e.g., CD56+ or CD16+). In some embodiments, the target cell is a T cell. In some embodiments, the target cell is a CD4+ T cell. In some embodiments, the target cell is a CD8+ T cell. In some embodiments, the a central T target cell is memory cell (e.g., CCR7+CD45RA−CD45RO+CD62L+CD27+). In some embodiments, the T cell is CCR7+. In some embodiments, the T cell is CD62L+. In some embodiments, the T cell is CD45RO+. Such positivity can be determined for example by flow cytometry compared to an isotype control or a cell population known to be negative for the specific marker. In some embodiments, the target cell is a myeloid cell. The myeloid cell lineage comprises neutrophils, eosinophil, basophils, monocytes, dendritic cells, and macrophages. In some embodiments, the therapeutic cell is an eosinophil, a basophil, a dendritic cell, a monocyte, a macrophage, a microglial cell, a Kupffer cell, or an alveolar macrophage.

The therapeutic cells described herein can be endogenous cells that have been isolated and enriched. In some embodiments, the therapeutic cells are derived from a subject. In some embodiments, the therapeutic cells are allogenic. Additionally, therapeutic cells can be derived from endogenous cells comprising pluripotent stem cells, hematopoietic stem cells, placental or fetal cells, from an adult human. The therapeutic cells can also be obtained from an established cell line or culture. In some embodiments, the therapeutic cells comprise cells derived from a cell line or established culture, wherein the cell line or established culture is derived from endogenous cells comprising pluripotent stem cells, hematopoietic stem cells, placental or fetal cells, from an adult human.

In certain embodiments, the target cells comprise adipose derived stem cells. In certain embodiments, the target cells comprise bone marrow derived stem cells. In certain embodiments, the stem cells. In certain embodiments, target cell population comprise mesenchymal stem cells.

One limitation of existing methods that use therapeutically active cells is low yields of suitable cells from primary sources (e.g., apheresis, individual donors) that can be subsequently genetically engineered. The methods described herein increase the absolute number and percentage as of certain T cell populations useful for making and producing therapeutic cell populations. The cell populations produced herein and suitable for genetic engineering and can comprise high levels of CD3 T cells. The population can comprise a CD45+ lymphocyte population that is greater than about 50% CD3+ T cells, greater than about 55% CD3+ T cells greater than about 60% CD3+ T cells, greater than about 65% CD3+ T cells, greater than about 70% CD3+ T cells, greater than about 75% CD3+ T cells, greater than about 80% CD3+ T cells, or greater than about 85% CD3+ T cells.

The methods described herein can produce populations of enriched target cells for genetic engineering that exceed about 1×10⁶, about 2×10⁶, about 5×10⁶, about 1×10⁶, about 1×10⁷, about 2×10⁷, about 5×10⁷, about 1×10⁸, about 2×10⁸, about 5×10⁸, about 1×10⁹, about 2×10⁹, about 1×10¹⁰, about 2×10¹⁰, or about 5×10¹⁰ or more. The methods described herein can produce populations of CD45+ lymphocytes cells that exceed about 1×10⁶, about 2×10⁶, about 5×10⁶, about 1×10⁶, about 1×10⁷, about 2×10⁷, about 5×10⁷, about 1×10⁸, about 2×10⁸, about 5×10⁸, about 1×10⁹, about 2×10⁹, about 1×10¹⁰, about 2×10¹⁰, or about 5×10¹⁰ or more. The methods described herein can produce populations of CD3+ T lymphocytes cells that exceed about 1×10⁶, about 2×10⁶, about 5×10⁶, about 1×10⁶, about 1×10⁷, about 2×10⁷, about 5×10⁷, about 1×10⁸, about 2×10⁸, about 5×10⁸, about 1×10⁹, about 2×10⁹, about 1×10¹⁰ about 2×10¹⁰, or about 5×10¹⁰ or more.

In some cases, the methods described herein can produce a population of enriched cells that comprises an increased number of white blood cells in the cell population when compared to a buffy cell coat population isolated from a sample by density gradient centrifugation. In some cases, the population of enriched cells can contain a number of white blood cells in the cell population that is at least 2 times more, 2.5 times more, 3 times more, 4 times more, or 5 times more than a number of white blood cells in the buffy coat cell population. In some cases, the population of enriched cells can contain a number of white bloods cell in the cell population that is at least 2 times less, 2.5 times less, 3 times less, 4 times less, or 5 times less than the number of white blood cells in the buffy coat cell population.

In some cases, the methods described herein produce a population of enriched cells that comprise an increased number of T cells when compared to a buffy coat cell population isolated from a sample by density gradient centrifugation. In some cases, the population of enriched cells can contain a number of T cells that is at least 2 times more, 2.5 times more, 3 times more, 4 times more, or 5 times more than the number of T cells in the buffy coat cell population. In some cases, the population of enriched cells can contain a number of T cells that is 2 times less, 2.5 times less, 3 times less, 4 times less, or 5 times less than the number of T cells in the buffy coat cell population.

In some cases, the methods described herein produce a population of enriched cells that comprises a lessor ratio of red blood cells to T cells when compared to the buffy coat cell population produced by density gradient centrifugation. In some cases, the population of cells comprises a ratio of red blood cells to T cells in the buffy coat cell population that is at least 5 times less, 4 times less, 3 times less, 2.5 times less, or 2 times less than a ratio of red blood cells to T cells in the buffy coat cell population. In some cases, the enriched cell population comprises a ratio of red blood cells to T cells in the buffy coat cell population that is at least 2 times more, 2.5 times more, 3 times more, 4 times more, or 5 times more than the buffy coat cell population.

In some cases, the methods described herein produce a population of enriched cells that comprises a lessor ratio of platelets to T cells when compared to the buffy coat cell population produced by density gradient centrifugation. In some cases, the enriched cell population comprises a ratio of platelets to T cells in the cell population that is at least 5 times less, 4 times less, 3 times less, 2.5 times less, or 2 times less than a ratio of platelets to T cells in the buffy coat cell population. In some cases, the enriched cell population comprises a ratio of platelets to T cells in the cell population that is at least 5 times more, 4 times more, 3 times more, 2.5 times more, or 2 times more than a ratio of platelets to T cells in the buffy coat cell population.

In some cases, the methods described herein produce a population of enriched cells that comprises a lessor percentage of senescent cells when compared to the buffy coat cell population produced by density gradient centrifugation. In some cases, the enriched cell population comprises a percentage of senescent cells that is at least 10% less, 9% less, 8% less, 7% less, 6% less, 5% less, 4% less, 3% less, 2.5% less, or 2% less than the percentage of senescent cells in the buffy coat cell population. In some cases, the enriched cell population comprises a percentage of senescent cells that is at least 10% more, 9% more, 8% more, 7% more, 6% more 5% more, 4% more, 3% more, 2.5% more, or 2% more than a percentage of senescent cells in the cell population.

In some cases, the methods described herein produce a population of enriched cells that comprises a lessor percentage of exhausted cells when compared to the buffy cell coat population produced by gradient density centrifugation. In some cases, the enriched cell population comprises a percentage of exhausted cells in the cell population that is at least 10% less, 9% less, 8% less, 7% less, 6% less, 5% less, 4% less, 3% less, 2.5% less, or 2% less than a percentage of exhausted cells in the buffy coat cell population. In some cases, the enriched cell population comprises a percentage of exhausted cells in the cell population that is at least 10% more, 9% more, 8% more, 7% more, 6% more, 5% more, 4% more, 3% more, 2.5% more, or 2% more than a percentage of exhausted cells in the buffy coat cell population.

In some cases, the methods describe herein produce an enriched population of cells that comprises a lessor percentage of T effector memory cells that express CD45Ra in the cell population when compared to the buffy coat cell population produced by gradient density centrifugation. In some cases, the enriched cell population comprises a percentage of T effector memory cells that express CD45Ra in the cell population that is at least 10% less, 9% less, 8% less, 7% less, 6% less, 5% less, 4% less, 3% less, 2.5% less, or 2% less than a percentage of T effector memory cells that express CD45Ra in the buffy coat cell population. In some cases, the methods described herein produce an enriched population of cells that comprises a lessor percentage of T effector memory cells that express CD45Ra in the cell population that is at least 10% more, 9% more, 8% more, 7% more, 6% more, 5% more, 4% more, 3% more, 2.5% more, or 2% more than a percentage of T effector memory cells that express CD45Ra in the buffy coat cell population.

In some cases, the methods herein produce an enriched population of cells that comprises a greater percentage of T central memory cells when compared to a buffy coat cell population produced by gradient density centrifugation. In some cases, the enriched cell population comprises a percentage of T central memory cells that is at least 6% more, 7% more, 8% more, 9% more, 10% more, 15% more, 20% more, 30% more, or 40% more than in the buffy coat cell population. In some cases, the enriched cell population comprises a percentage of T central memory cells that is at least 6% less, 7% less, 8% less, 9% less, or 10% less than in the buffy coat cell population.

In some cases, the methods described herein can produce a greater percentage of cells in the cell population that are T central memory cells or T effector memory cells than a percentage of cells in the buffy coat cell population that are T central memory cells or T effector memory cells. In some cases, the cell population contains a percentage of cells in the cell population that are T central memory cells or T effector memory cells that is at least 10% higher, 15% higher, 20% higher, 25% higher, 30% higher, or 40% higher than the percentage of cells in the buffy coat cell population that are T central memory cells or T effector memory cells.

The cells enriched by the methods herein can comprise a high level of viability before genetic engineering, in excess of 60%, 65%, 70%, 75%, 80%, 85%, 90%, 95% or more.

Target cells can also be subjected to buffer exchange during enrichment and the resulting populations can be resuspended in a variety of buffers and or media useful for cell culture or downstream processing. Such buffers and or media may be isotonic and/or pH buffered to reflect a physiological osmolality or pH. Such buffers and or media may also comprise one or more energy sources such as glucose or dextrose, and/or vitamin and/or mineral supplements Specific buffers or media include without limitation, phosphate buffered saline, Hank's buffered salt solution, ringer buffer (with or without glucose), RPMI, DMEM, buffers or media comprising animal serum 5%, 10%, 15% or 20% (human or other animal), buffers or media comprising an appropriate serum substitute or formulated without serum (e.g., X-VIVO 10TM, X-VIVO 15TM, X-VIVO 20TM).

After enrichment target cells can be cultured in an appropriate medium or buffer for at least 1 hour, 2 hours, 4 hours, 8 hours, 16 hours, 24 hours, 2 days, 3 days, 4 days, 5 days, 6 days, 7 days, 8 days, 9 days, 10 days, or more. In certain embodiments, the culturing of enriched target cells is for no more than 15 days, 10 days, 9 days, 8 days, 7 days, 6 days, 5 days, 4 days, or 3 days. Such culturing can be carried out at 37 degrees Celsius under an enriched CO₂ environment (e.g., 1%, 2%, 5%, 10% or more CO₂). In certain embodiments, cells that are enriched by the methods herein are enriched in a sterile and/or GMP facility.

The methods described herein can comprise an additional step of activating the cells before genetically engineering them. For example, primary cells may be induced to enter the cell cycle in order for a gene to integrate into the genome of the target cell. The methods can comprise an additional activation step after enrichment. In certain embodiments, the activation step comprises contacting enriched target cells with IL-15 and/or IL-7. In certain embodiments, the activation step comprises contacting enriched target cells with and activating agent. In certain embodiments, the activating agent comprises anti-CD3 antibody and/or CD28 antibody. In certain embodiments, the activating agent comprises anti-CD3 antibody and/or anti-CD28 antibody that is conjugated to a solid support. In certain embodiments, the solid support is a magnetic bead. In certain embodiments, the contacting the population of large cells with the anti-CD3 antibody or the anti-CD28 antibody conjugated to a solid support further comprises affinity enrichment of leukocytes expressing CD3 or CD28. In certain embodiments, the activation step comprises contacting enriched target cells with IL-2, IL-15, IL-7, anti-CD3 antibody and/or CD28 antibody. IL-15 and IL-17 are cytokines that support activation and expansion of T cells. IL-15 can be applied at about or at least about 5 ng/ml, 10 ng/mL, 15 ng/ml, 20 ng/mL, 25 ng/ml, 30 ng/mL, 35 ng/mL, 40 ng/mL, 45 ng/mL, 50 ng/mL, 75 ng/mL, or 100 ng/mL or more. IL-15 can be applied from about 1 ng/mL to about 100 ng/mL, from about 5 ng/ml to about 100 ng/ml, from about 10 ng/mL to about 100 ng/mL, from about 25 ng/mL to about 100 ng/mL, from about 50 ng/ml to about 100 ng/mL, from about 1 ng/mL to about 75 ng/mL, from about 5 ng/ml to about 75 ng/mL, from about 10 ng/ml to about 75 ng/mL, from about 25 ng/ml to about 75 ng/ml, or from about 40 ng/ml to about 60 ng/mL. IL-7 can be applied at about or at at least about 5 ng/mL, 10 ng/mL, 15 ng/ml, 20 ng/ml, 25 ng/ml, 30 ng/mL, 35 ng/mL, 40 ng/mL, 45 ng/mL, 50 ng/mL, 75 ng/ml, or 100 ng/ml or more. IL-15 can be applied from about 1 ng/mL to about 100 ng/mL, from about 5 ng/ml to about 100 ng/ml, from about 10 ng/mL to about 100 ng/mL, from about 25 ng/ml to about 100 ng/mL, from about 50 ng/ml to about 100 ng/mL, from about 1 ng/ml to about 75 ng/mL, from about 5 ng/mL to about 75 ng/ml, from about 10 ng/mL to about 75 ng/mL, from about 25 ng/ml to about 75 ng/ml, or from about 40 ng/ml to about 60 ng/mL. In certain embodiments, the activating agent comprises one or more of an anti-CD3 antibody, an anti-CD28 antibody, an anti-CD137 antibody, an anti-CD2 antibody, an anti-CD35 antibody, interleukin-2, interleukin-7, or interleukin-15, interleukin-21, interleukin-6, TGFbeta, CD40 ligand, PMA/Ionomycin, Concanavalin A, Pokeweed mitogen, phytohemagglutinin, such activating agents can be appropriately used to activate NK cell, B-cells, or T-cells, according to the methods described herein.

Enriched target cells can be contacted to activating agents for at least 1, 2, 3, 4, 5, 6, 7, or more days before genetic engineering.

### Genetic Engineering

The cell populations produced by the methods described herein are suitable for genetic engineering. Such genetic engineering results in enriched target cells comprising an exogenous nucleic acid. In certain embodiments, the nucleic acid comprises a promoter operatively coupled to a coding region for a gene of interest allowing transcription and translation of the gene of interest under suitable circumstances. The promoter may be an inducible promoter, a tissue specific, or a universal promoter. The gene of interest may also be coupled to additional regulatory elements such as a polyadenylation signal or one or more enhancers. The gene of interest may encode any one or more of an immunoglobulin, a chimeric antigen receptor, a T cell receptor, a cytokine, or a chemokine. In certain embodiments, the gene of interest encodes an immunoglobulin. In certain embodiments, the gene of interest encodes a chimeric antigen receptor. In certain embodiments, the gene of interest encodes a T cell receptor. In certain embodiments, the gene of interest encodes a cytokine, or a chemokine. In certain embodiments, the gene of interest is a CRISPR construct comprising a target strand and a guide strand.

The compositions, methods, and systems disclosed provided collected target cell products (e.g. cell populations) that facilitate the generation of chimeric antigen receptor (CAR) T cells. Chimeric antigen receptor (CAR) T cell immunotherapy is a highly effective form of adoptive cell therapy, as demonstrated by the remission rates in patients with B cell acute lymphoblastic leukemia or large B cell lymphoma, which have supported FDA approvals.

Methods for making and using CAR T cells are known in the art. Procedures have been described in, for example, U.S. Pat. Nos. 9,629,877; 9,328,156; 8,906,682; US 2017/0224789; US 2017/0166866; US 2017/0137515; US 2016/0361360; US 2016/0081314; US 2015/0299317; and US 2015/0024482; each of which is incorporated by reference herein in its entirety.

CAR T cells made using the methods discussed herein may be used in treating patients for leukemia, e.g., acute lymphoblastic leukemia using procedures well established in the art of clinical medicine and, in these cases, the CAR may recognize CD19 or CD20 as a tumor antigen. The method may also be used for solid tumors, in which case antigens recognized may include CD22; RORI; mesothelin; CD33/IL3Ra; c-Met; PSMA; Her2/Neu; CD38, Glycolipid F77; EGFRvIII; GD-2; NY-ESO-1; MAGE A3; and combinations thereof. With respect to autoimmune diseases, CAR T cells may be used to treat rheumatoid arthritis, lupus, multiple sclerosis, ankylosing spondylitis, type 1 diabetes or vasculitis.

In certain embodiments the methods described herein are useful for generating a lymphocyte expressing a heterologous gene for use in treating a hematological cancer or a solid tumor. In some embodiments, the cancer is a bladder cancer, breast cancer, colon and rectal cancer, endometrial cancer, kidney cancer, leukemia, liver cancer, lung cancer, melanoma, non-Hodgkin lymphoma, pancreatic cancer, prostate cancer or thyroid cancer.

In certain embodiments, the method described herein can be used to produce T cells comprising a chimeric antigen receptor. In certain embodiments, the method describe herein can be used to produce axicabtagene ciloleucel. In certain embodiments, the method described herein can be used to produce brexucabtagene autoleucel. In certain embodiments, the method described herein can be used to produce tisagenlecleucel. In certain embodiments, the method described herein can be used to produce lisocabtagene maraleucel or idecabtagene vicleucel.

The methods described herein are suitable for genetically engineering an enriched target cell population using an activating agent, such as a virus that comprises a gene of interest. The enriched target cell populations may be contacted with a virus that the comprises a gene of interest. In certain embodiments, the virus is a lentivirus, adenovirus, or adeno-associated virus. In certain embodiments, the virus is a lentivirus. In certain embodiments, the virus is an adenovirus. In certain embodiments, the virus is an adeno-associated virus.

The target cells produced herein creates cells populations with ability to be transduced as a high level of efficiency by a viral vector. The transduction efficacy of the target cells produced by the method described herein can be at least about 50%, 55%, 60%, 65%, 70%, 75%, 80%, or 85% three-days after transduction. The transduction efficacy of the target cells produced by the method described herein can be at least about 50%, 55%, 60%, 65%, 70%, 75%, 80%, or 85% six-days after transduction.

For viral genetic engineering the cell populations can be contacted with a virus at a predetermined multiplicity of infection (MOI). In some embodiments the MOI is about 5 to about 200. In some embodiments the MOI is about 5 to about 10, about 5 to about 20, about 5 to about 25, about 5 to about 30, about 5 to about 40, about 5 to about 50, about 5 to about 75, about 5 to about 100, about 5 to about 200, about 10 to about 20, about 10 to about 25, about 10 to about 30, about 10 to about 40, about 10 to about 50, about 10 to about 75, about 10 to about 100, about 10 to about 200, about 20 to about 25, about 20 to about 30, about 20 to about 40, about 20 to about 50, about 20 to about 75, about 20 to about 100, about 20 to about 200, about 25 to about 30, about 25 to about 40, about 25 to about 50, about 25 to about 75, about 25 to about 100, about 25 to about 200, about 30 to about 40, about 30 to about 50, about 30 to about 75, about 30 to about 100, about 30 to about 200, about 40 to about 50, about 40 to about 75, about 40 to about 100, about 40 to about 200, about 50 to about 75, about 50 to about 100, about 50 to about 200, about 75 to about 100, about 75 to about 200, or about 100 to about 200. In some embodiments the MOI is about 5, about 10, about 20, about 25, about 30, about 40, about 50, about 75, about 100, or about 200. In some embodiments the MOI is at least about 5, about 10, about 20, about 25, about 30, about 40, about 50, about 75, or about 100. In some embodiments the MOI is at most about 10, about 20, about 25, about 30, about 40, about 50, about 75, about 100, or about 200.

In some embodiments the MOI for a lentivirus comprising an exogenous non-viral nucleic acid is about 5 to about 200. In some embodiments the MOI for a lentivirus comprising an exogenous non-viral nucleic acid is about 5 to about 10, about 5 to about 20, about 5 to about 25, about 5 to about 30, about 5 to about 40, about 5 to about 50, about 5 to about 75, about 5 to about 100, about 5 to about 200, about 10 to about 20, about 10 to about 25, about 10 to about 30, about 10 to about 40, about 10 to about 50, about 10 to about 75, about 10 to about 100, about 10 to about 200, about 20 to about 25, about 20 to about 30, about 20 to about 40, about 20 to about 50, about 20 to about 75, about 20 to about 100, about 20 to about 200, about 25 to about 30, about 25 to about 40, about 25 to about 50, about 25 to about 75, about 25 to about 100, about 25 to about 200, about 30 to about 40, about 30 to about 50, about 30 to about 75, about 30 to about 100, about 30 to about 200, about 40 to about 50, about 40 to about 75, about 40 to about 100, about 40 to about 200, about 50 to about 75, about 50 to about 100, about 50 to about 200, about 75 to about 100, about 75 to about 200, or about 100 to about 200. In some embodiments the MOI for a lentivirus comprising an exogenous non-viral nucleic acid is about 5, about 10, about 20, about 25, about 30, about 40, about 50, about 75, about 100, or about 200. In some embodiments the MOI for a lentivirus comprising an exogenous non-viral nucleic acid is at least about 5, about 10, about 20, about 25, about 30, about 40, about 50, about 75, or about 100. In some embodiments the MOI for a lentivirus comprising an exogenous non-viral nucleic acid is at most about 10, about 20, about 25, about 30, about 40, about 50, about 75, about 100, or about 200.

In certain embodiments, the viral vector (e.g. adenovirus, lentivirus, AAV) comprises a heterologous nucleic acid. The heterologous nucleic acid may comprise a sequence encoding a polypeptide of interest. The polypeptide of interest may be a chimeric antigen receptor, a T cell receptor, a polypeptide comprising an immunoglobulin domain, a cytokine, a chemokine, or any other polypeptide such as a receptor. In other embodiments, the heterologous nucleic acid comprises a sequence of an siRNA or miRNA.

The cells described herein are also suitable for genetic engineering by other methods such as by electroporation. The cells may also be suitable for genetic engineering by a compression such as by methods and devices described in WO2020/117856 A1.

One advantage of the methods described herein is the provision of transgenic cells at earlier time-points allowing for more efficient production of genetically engineered cells for research or therapeutic use. In certain embodiments, enriched target cells can be harvested for therapeutic or research purposes 1, 2, 3, 4, 5, or more days post engineering. In certain embodiments, enriched target cells can be harvested for therapeutic or research use about 5 days post engineering to about 17 days post engineering. In certain embodiments, enriched target cells can be harvested about 5 days post engineering to about 7 days post engineering, about 5 days post engineering to about 8 days post engineering, about 5 days post engineering to about 9 days post engineering, about 5 days post engineering to about 10 days post engineering, about 5 days post engineering to about 11 days post engineering, about 5 days post engineering to about 12 days post engineering, about 5 days post engineering to about 13 days post engineering, about 5 days post engineering to about 14 days post engineering, about 5 days post engineering to about 15 days post engineering, about 5 days post engineering to about 16 days post engineering, about 5 days post engineering to about 17 days post engineering, about 7 days post engineering to about 8 days post engineering, about 7 days post engineering to about 9 days post engineering, about 7 days post engineering to about 10 days post engineering, about 7 days post engineering to about 11 days post engineering, about 7 days post engineering to about 12 days post engineering, about 7 days post engineering to about 13 days post engineering, about 7 days post engineering to about 14 days post engineering, about 7 days post engineering to about 15 days post engineering, about 7 days post engineering to about 16 days post engineering, about 7 days post engineering to about 17 days post engineering, about 8 days post engineering to about 9 days post engineering, about 8 days post engineering to about 10 days post engineering, about 8 days post engineering to about 11 days post engineering, about 8 days post engineering to about 12 days post engineering, about 8 days post engineering to about 13 days post engineering, about 8 days post engineering to about 14 days post engineering, about 8 days post engineering to about 15 days post engineering, about 8 days post engineering to about 16 days post engineering, about 8 days post engineering to about 17 days post engineering, about 9 days post engineering to about 10 days post engineering, about 9 days post engineering to about 11 days post engineering, about 9 days post engineering to about 12 days post engineering, about 9 days post engineering to about 13 days post engineering, about 9 days post engineering to about 14 days post engineering, about 9 days post engineering to about 15 days post engineering, about 9 days post engineering to about 16 days post engineering, about 9 days post engineering to about 17 days post engineering, about 10 days post engineering to about 11 days post engineering, about 10 days post engineering to about 12 days post engineering, about 10 days post engineering to about 13 days post engineering, about 10 days post engineering to about 14 days post engineering, about 10 days post engineering to about 15 days post engineering, about 10 days post engineering to about 16 days post engineering, about 10 days post engineering to about 17 days post engineering, about 11 days post engineering to about 12 days post engineering, about 11 days post engineering to about 13 days post engineering, about 11 days post engineering to about 14 days post engineering, about 11 days post engineering to about 15 days post engineering, about 11 days post engineering to about 16 days post engineering, about 11 days post engineering to about 17 days post engineering, about 12 days post engineering to about 13 days post engineering, about 12 days post engineering to about 14 days post engineering, about 12 days post engineering to about 15 days post engineering, about 12 days post engineering to about 16 days post engineering, about 12 days post engineering to about 17 days post engineering, about 13 days post engineering to about 14 days post engineering, about 13 days post engineering to about 15 days post engineering, about 13 days post engineering to about 16 days post engineering, about 13 days post engineering to about 17 days post engineering, about 14 days post engineering to about 15 days post engineering, about 14 days post engineering to about 16 days post engineering, about 14 days post engineering to about 17 days post engineering, about 15 days post engineering to about 16 days post engineering, about 15 days post engineering to about 17 days post engineering, or about 16 days post engineering to about 17 days post engineering. In certain embodiments, enriched target cells can be harvested about 5 days post engineering, about 7 days post engineering, about 8 days post engineering, about 9 days post engineering, about 10 days post engineering, about 11 days post engineering, about 12 days post engineering, about 13 days post engineering, about 14 days post engineering, about 15 days post engineering, about 16 days post engineering, or about 17 days post engineering. In certain embodiments, enriched target cells can be harvested at least about 5 days post engineering, about 7 days post engineering, about 8 days post engineering, about 9 days post engineering, about 10 days post engineering, about 11 days post engineering, about 12 days post engineering, about 13 days post engineering, about 14 days post engineering, about 15 days post engineering, or about 16 days post engineering. In certain embodiments, enriched target cells can be harvested at most about 7 days post engineering, about 8 days post engineering, about 9 days post engineering, about 10 days post engineering, about 11 days post engineering, about 12 days post engineering, about 13 days post engineering, about 14 days post engineering, about 15 days post engineering, about 16 days post engineering, or about 17 days post engineering. Post harvesting cells can be subject to additional enrichment steps including washing, concentrating, buffer exchange, or transport to proper facilitates for administration.

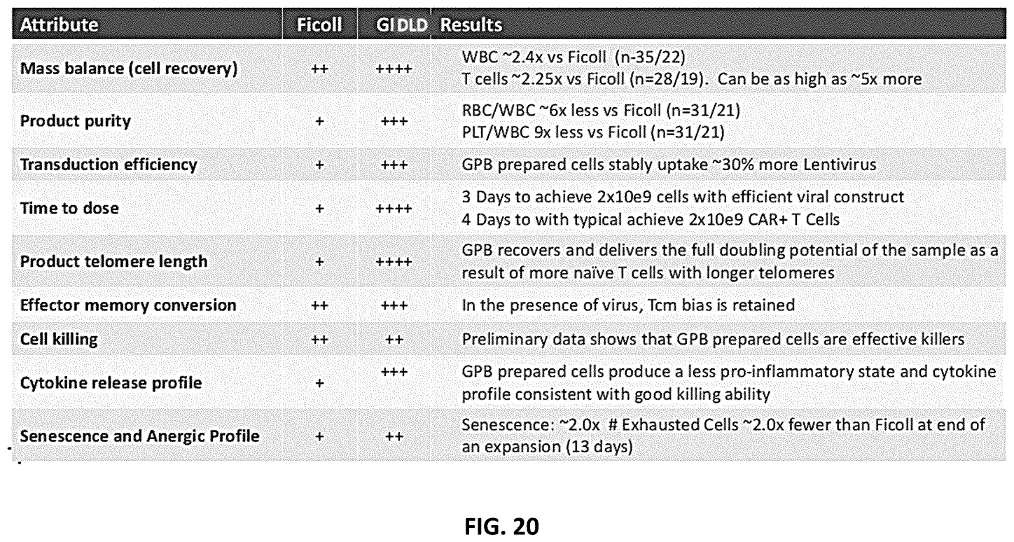

The cells produced herein comprise many beneficial properties that are desirable for transfection with therapeutic vectors, production of therapeutic doses, and downstream avoidance of certain undesirable cell phenotypes after transfection or expansion. These beneficial properties are shown in FIG. 20.

In some cases, the methods described herein produce a population of cells that exhibits an increased ability to expand in culture, wherein the cells are expanded before or after being genetically modified when compared to a population of cells produced by a density gradient centrifugation method. In some cases, the population of cells exhibits at least about 1.1-fold, 1.5-fold, 2-fold, 3-fold, 4-fold, 5-fold, or 10-fold increase in ability to expand in culture, wherein the cells are expanded before or after being genetically modified when compared to a population of cells produced by a density gradient centrifugation method. In some cases, the cell population is capable of expanding to comprise at least 2×10e9 T cells comprising the heterologous DNA in at least 5% less, 10% less, 15% less, 20% less 25% less or 30% less time than the buffy coat cell population produced by gradient density centrifugation.

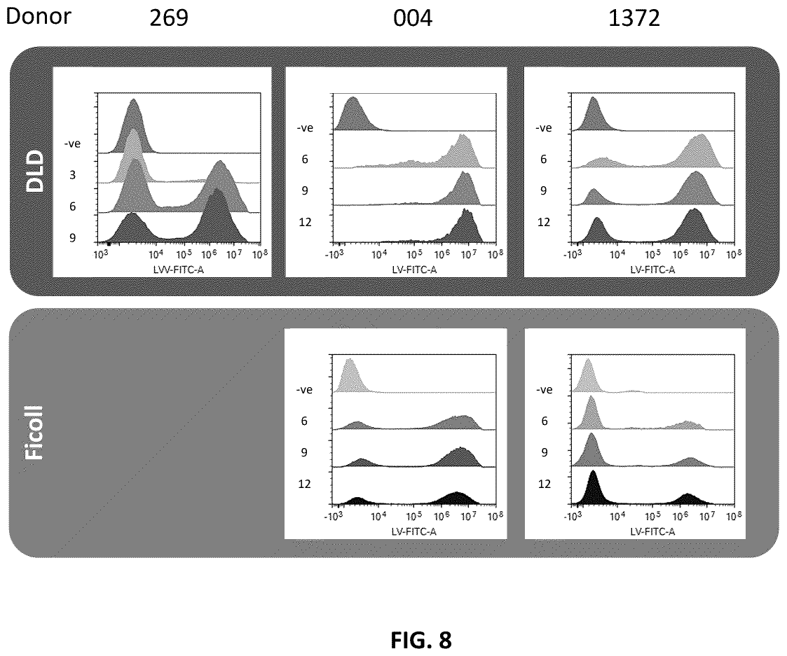

In some cases, the methods described herein produce a population of cells that exhibit an increase in ability to readily integrate a lentiviral vector (i.e. at least some portion of the nucleic acid of the lentivirus is inserted into the cells' genomes or exosomes) when compared to a population of cells produced by a density gradient centrifugation method. In some cases, the population of cells exhibit at least about 5%, 10%, 15%, 25%, 30%, 50%, 100%, 200%, 300%, 400%, or 500% increased ability to readily integrate a lentiviral vector when compared to a population of cells produced by a density gradient centrifugation method. In some examples, it has been shown that the methods described herein produce a population of cells that have a 30% increased ability to readily integrate lentivirus. See FIG. 8.

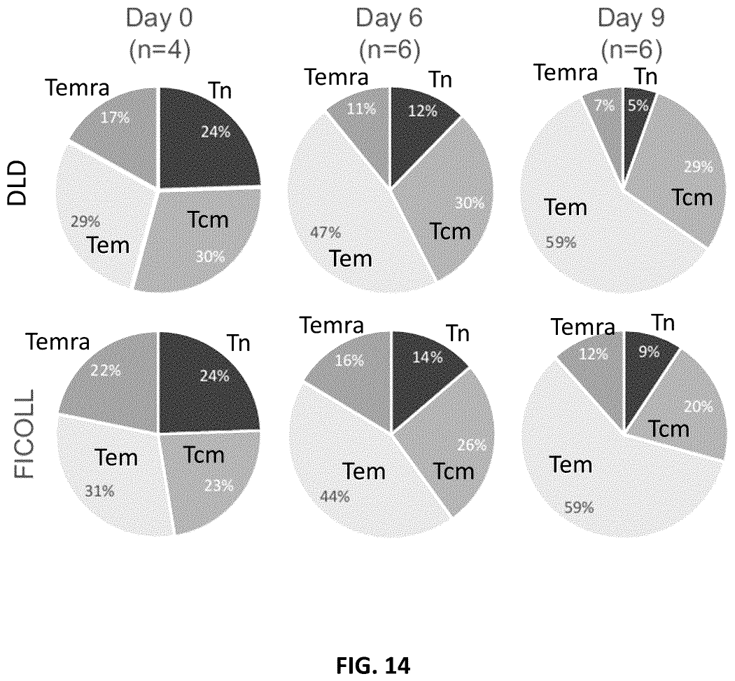

In some cases, the methods described herein produce a population of cells that exhibit an increase in ability to retain T cell memory composition while in cell culture when compared to a population of cells produced by a density gradient centrifugation method. In some cases, the population of cells retains its relative T cell memory composition for at least about 1, 2, 3, 4, 5, 6, 7, 8, 9, 10, 11, 12, 13, 14, 15, 16, 17, 18, 19, or 20 days longer than a population of cells produced by a density gradient centrifugation method. For example, it has been shown that DLD methods produce cell populations that retain their relative memory T cell populations longer than other methods including Ficoll. See FIG. 14.

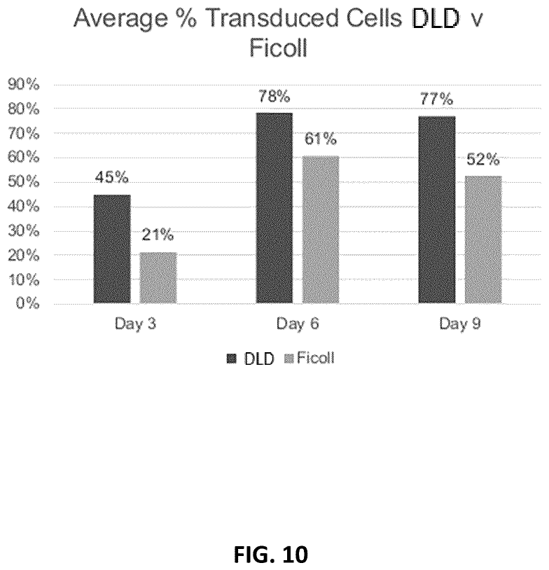

In some cases, the methods described herein produce a population of cells that exhibit an increase in receptivity to viral transduction when compared to a population of cells produced by a density gradient centrifugation method. In some cases, the population of cells exhibit at least about 5%, 10%, 15%, 25%, 30%, 50%, 100%, 200%, 300%, 400%, or 500% increased receptivity to viral transduction when compared to a population of cells produced by a density gradient centrifugation method. For example, it has been shown that DLD methods produce a population of cells that are about between 20-40% more receptive to viral transduction than cells produced using Ficoll or other methods. See FIG. 10.

In some cases, the methods described herein produce a population of cells that exhibit an increase in mean absolute telomere length when compared to a population of cells produced by a density gradient centrifugation method. In some cases, the population of cells exhibit at least about 5%, 10%, 15%, 25%, 30%, 50%, 100%, 200%, 300%, 400%, or 500% increased mean absolute telomere length when compared to a population of cells produced by a density gradient centrifugation method.

In some cases, the methods described herein produce a population of cells that exhibit an increase in ability to retain a relative population of less differentiated naïve and central memory cells while in cell culture when compared to a population of cells produced by a density gradient centrifugation method. In some cases, the population of cells retains its relative population of less differentiated naïve and central memory cells while in cell culture for at least about 1, 2, 3, 4, 5, 6, 7, 8, 9, 10, 11, 12, 13, 14, 15, 16, 17, 18, 19, or 20 days longer than a population of cells produced by a density gradient centrifugation method.

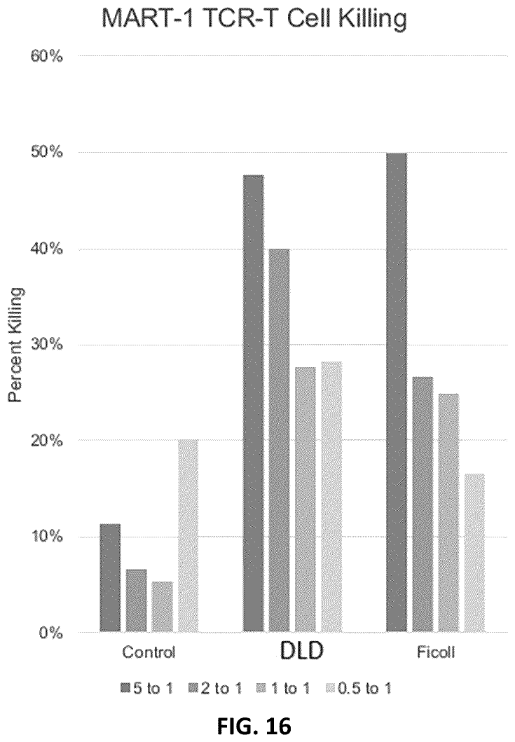

In some cases, the methods described herein produce a population of cells that exhibit an increase in functional killing capacity when compared to a population of cells produced by a density gradient centrifugation method. In some cases, the population of cells exhibit at least about 5%, 10%, 15%, 25%, 30%, 50%, 100%, 200%, 300%, 400%, or 500% increased functional killing capacity when compared to a population of cells produced by a density gradient centrifugation method. For example, it has been shown that DLD methods produce cells that have at least 30% greater killing capacity as compared to cells produced using Ficoll methods, when seeded at 2 cells per targeted cell for killing. See FIG. 16.

Flow cytometry may be used to determine the percentage of target cells within a population that are expressing a polypeptide. In some cases, the methods described herein produce a population of T-cells wherein at least 70%, 80%, 90%, 95%, 97%, 98%, or 99% of the T-cells express a polynucleotide/polypeptide as determined by flow cytometry.

In some cases, the methods described herein produce a population of cells that exhibit an increase in IFN gamma expression when compared to a population of cells produced by a density gradient centrifugation method. In some cases, the population of cells exhibits at least about 1.1-fold, 1.5-fold, 2-fold, 3-fold, 4-fold, 5-fold, or 10-fold increase in IFN gamma expression when compared to a population of cells produced by a density gradient centrifugation method. In some cases, the population of cells exhibits at least about a 1% increase, a 5% increase, a 10% increase, a 15% increase, a 20% increase, or a 50% increase in IFN gamma expression when compared to a population of cells produced by a density gradient centrifugation method. In some cases, the increase in IFN gamma expression is apparent 0, 3, 6, 9, 13, or 16 days after being produced by the methods and systems described herein.

In some cases, the methods described herein produce a population of cells that exhibit an increase in GM-CSF expression when compared to a population of cells produced by a density gradient centrifugation method. In some cases, the population of cells exhibits at least about 1.1-fold, 1.5-fold, 2-fold, 3-fold, 4-fold, 5-fold, or 10-fold increase in GM-CSF expression when compared to a population of cells produced by a density gradient centrifugation method. In some cases, the increase in GM-CSF expression is apparent 0, 3, 6, 9, 13, or 16 days after being produced by the methods and systems described herein.

In some cases, the methods described herein produce a population of cells that exhibit an increase in TNFa expression when compared to a population of cells produced by a density gradient centrifugation method. In some cases, the population of cells exhibits at least about 5%, 10%, 15%, 25%, 30%, 50%, 100%, 200%, 300%, 400%, or 500% increase in TNFa expression when compared to a population of cells produced by a density gradient centrifugation method. In some cases, the increase in TNFa expression is apparent 0, 3, 6, 9, 13, or 16 days after being produced by the methods and systems described herein.

In some cases, the methods described herein produce a population of cells, wherein most of the cells of the cell population are viable. In some cases, the population of cells comprises of at least 70%, 75%, 80%, 85%, 90%, 95%, 96%, 97%, 98%, or 99% of viable cells. In some cases, the methods described herein produce a population of cells that exhibit an increase in viability when compared to a population of cells produced by a density gradient centrifugation method. In some cases, the population of cells exhibits at least about 5%, 10%, 15%, 25%, 30%, 50%, 100%, 200%, 300%, 400%, or 500% increase in viability when compared to a population of cells produced by a density gradient centrifugation method. In some cases, the methods herein produce a population of genetically modified leukocytes that consists of at least 70%, 75%, 80%, 85%, 90%, 95%, 96%, 97%, 98%, or 99% viable genetically modified leukocytes.

In some cases the methods described herein produce a composition of genetically engineered leukocyte that comprises of at least 1×10e9 T cells, 2×10e9 T cells, 3×10e9 T cells, 5×10e9 T cells, 7×10e9 T cells, or 9×10e9 T cells. In some cases, the methods described herein produce a genetically engineered leukocyte composition in which at least 50%, 60%, 70%, 75%, 80%, 85%, 90%, 95%, 97%, 98%, or 99% of the T cells within the composition are either T central memory cells or T effector T cells after 9 days of culturing.

In some cases, the methods described herein produce a population of cells that exhibit an increase in ability to readily integrate a lentiviral vector when compared to a population of cells produced by a density gradient centrifugation method.

In some cases, the methods described herein produce a population of cells that exhibit a decrease in time required to expand in culture to produce a single therapeutic dose equivalent of cells when compared to a population of cells produced by a density gradient centrifugation method. In some cases, the population of cells require about 1, 2, 3, 4, 5, 6, 7, or 8 fewer days to expand in culture to produce a single therapeutic dose equivalent of cells when compared to a population of cells produced by a density gradient centrifugation method. In some cases, the cell population require about 5% less, 10% less, 15% less, 20% less, 25% less, or 30% less time to expand in culture to comprise at least 2×10e9 T cells comprising the heterologous DNA than the buffy coat cell population produced by a density gradient centrifugation method when both the cell population and the buffy coat cell population are transduced with a viral vector comprising the heterologous DNA.

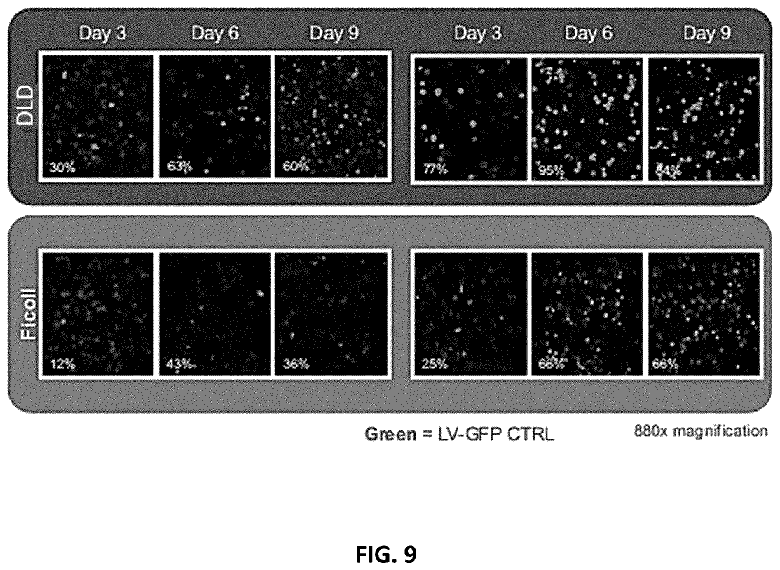

In some cases, the methods described herein produce a population of cells that exhibit a decrease in time required to express a gene delivered by a vector when compared to a population of cells produced by a density gradient centrifugation method. In some cases, the population of cells require about 1, 2, 3, 4, 5, 6, 7, or 8 fewer days to express a gene delivered by a vector when compared to a population of cells produced by a density gradient centrifugation method. For example, it has been shown that DLD methods produce cell populations that express a gene delivered by a vector faster than Ficoll or other methods. See FIG. 9.

Therapeutic dose equivalents may vary depending on the exact type of therapeutic but in some cases may be a at least about 1×10⁷, 2×10⁷, 3×10⁷, 4×10⁷, 5×10⁷, 1×10⁸, 2×10⁸, 3×10⁸, 4×10⁸, 5×10⁸, 1×10⁹, 2×10⁹, 3×10⁹, 4×10⁹, or 5×10⁹ total cells. Therapeutic doses may be about 1×10⁷, 2×10⁷, 3×10⁷, 4×10⁷, 5×10⁷, 1×10⁸, 2×10⁸, 3×10⁸, 4×10⁸, 5×10⁸, 1×10⁹, 2×10⁹, 3×10⁹, 4×10⁹, or 5×10⁹ transfected cells.

Therapeutic dose equivalents may vary depending on the exact type of therapeutic but in some cases may be a at least about 1×10⁷, 2×10⁷, 3×10⁷, 4×10⁷, 5×10⁷, 1×10⁸, 2×10⁸, 3×10⁸, 4×10⁸, 5×10⁸, 1×10⁹, 2×10⁹, 3×10⁹, 4×10⁹, or 5×10⁹ total cells. Therapeutic doses may be about 1×10⁷, 2×10⁷, 3×10⁷, 4×10⁷, 5×10⁷, 1×10⁸, 2×10⁸, 3×10⁸, 4×10⁸, 5×10⁸, 1×10⁹, 2×10⁹, 3×10⁹, 4×10⁹, or 5×10⁹ transfected cells.

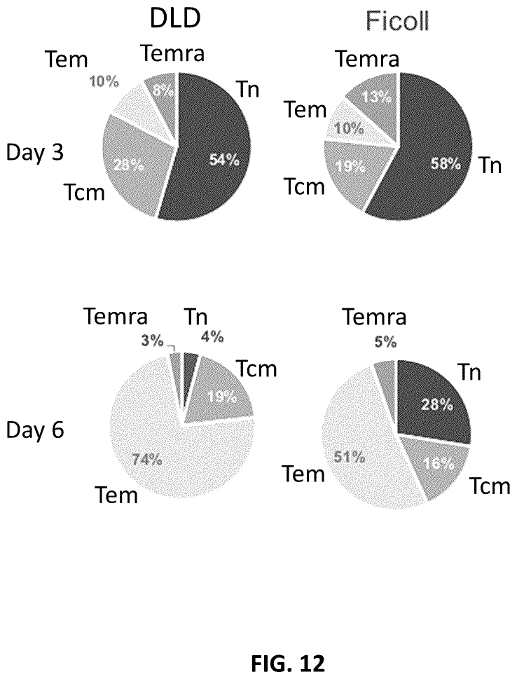

In some cases, the methods described herein produce a population of cells that exhibit a decrease in relative population of effector or Temra cells when compared to a population of cells produced by a density gradient centrifugation method. In some cases, the population of cells exhibits at least about 5%, 10%, 15%, 25%, 30%, 40%, 50%, 75%, or 90% decrease in relative population of effector or Temra cells when compared to a population of cells produced by a density gradient centrifugation method. For example, it has been shown that DLD methods produce cell populations with roughly 40% fewer Temra cells than cells produced using Ficoll or other methods, after treatment with a high integration lentivirus. See FIG. 12.

In some cases, the methods described herein produce a population of cells that exhibit a decrease in IL-1Ra expression when compared to a population of cells produced by a density gradient centrifugation method. In some cases, the population of cells exhibits at least about 5%, 10%, 15%, 25%, 30%, 40%, 50%, 60%, 75%, or 90% decrease in IL-1Ra expression when compared to a population of cells produced by a density gradient centrifugation method. In some cases, the decrease in IL-1Ra expression is apparent 0, 3, 6, 9, 13, or 16 days after being produced by the methods and systems described herein.

In some cases, the methods described herein produce a population of cells that exhibit a decrease in IL-6 expression when compared to a population of cells produced by a density gradient centrifugation method. In some cases, the population of cells exhibits at least about 5%, 10%, 15%, 25%, 30%, 40%, 50%, 60%, 75%, or 90% decrease in IL-6 expression when compared to a population of cells produced by a density gradient centrifugation method. In some cases, the decrease in IL-6 expression is apparent 0, 3, 6, 9, 13, or 16 days after being produced by the methods and systems described herein.

In some cases, the methods described herein produce a population of cells that exhibit a decrease in IL-13 expression when compared to a population of cells produced by a density gradient centrifugation method. In some cases, the population of cells exhibits at least about 5%, 10%, 15%, 25%, 30%, 40%, 50%, 60%, 75%, or 90% decrease in IL-13 expression when compared to a population of cells produced by a density gradient centrifugation method. In some cases, the decrease in IL-13 expression is apparent 0, 3, 6, 9, 13, or 16 days after being produced by the methods and systems described herein.

In some cases, the methods described herein produce a population of cells that exhibit a decrease in MCP-1 expression when compared to a population of cells produced by a density gradient centrifugation method. In some cases, the population of cells exhibits at least about a 5%, 10%, 15%, 20%, 25%, 30%, 40%, 50%, 60%, 75%, or 90% decrease in MCP-1 expression when compared to a population of cells produced by a density gradient centrifugation method. In some cases, the decrease in MCP-1 expression is apparent 0, 3, 6, 9, 13, or 16 days after being produced by the methods and systems described herein.

In some cases, the methods described herein produce a population of cells that exhibit a decrease in PD1 and Tim3 co-expression when compared to a population of cells produced by a density gradient centrifugation method. In some cases, the population of cells exhibits at least about a 5%, 10%, 15%, 20%, 25%, 30%, 40%, 50%, 60%, 75%, or 90% decrease in PD1 and Tim3 co-expression when compared to a population of cells produced by a density gradient centrifugation method. In some cases, the decrease in PD1 and Tim3 co-expression is apparent 0, 3, 6, 9, 13, or 16 days after being produced by the methods and systems described herein.

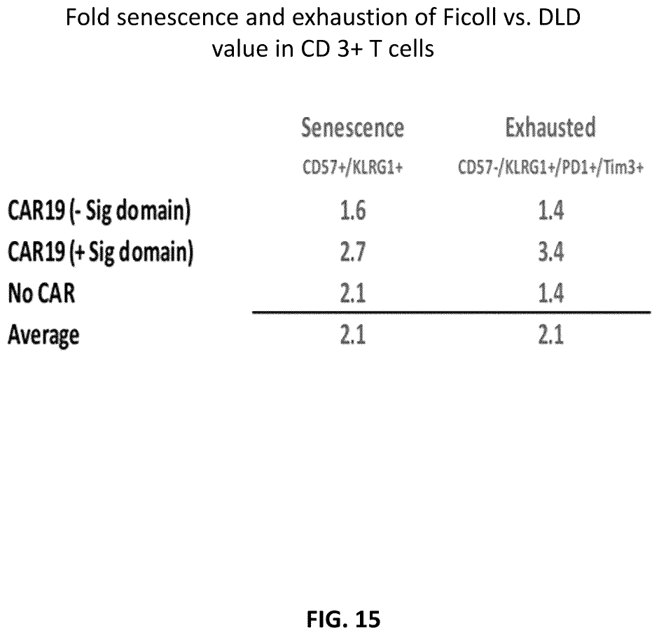

In some cases, the methods described herein produce a population of cells that exhibit a decrease in cell senescence or exhaustion when compared to a population of cells produced by a density gradient centrifugation method. In some cases, the population of cells exhibits at least about a 5%, 10%, 15%, 20%, 25%, 30%, 40%, 50%, 60%, 75%, or 90% decrease in cell senescence or exhaustion when compared to a population of cells produced by a density gradient centrifugation method. For example, it has been shown that DLD methods produce cells that show about 50% less PD1 and Tim3 co-expression compared to cells produced from Ficoll, suggesting they have lower senescence and exhaustion than cells produced using Ficoll. See FIG. 15.

In some cases, the methods described herein produce a population of cells that exhibit a decrease in propensity to trigger cytokine release syndrome when compared to a population of cells produced by a density gradient centrifugation method. In some cases, the population of cells exhibits at least about 5%, 10%, 15%, 20%, 25%, 30%, 40%, 50%, 60%, 75%, or 90% decrease in cell propensity to trigger cytokine release syndrome when compared to a population of cells produced by a density gradient centrifugation method.

In some cases, the methods described herein produce a population of cells that exhibit a decrease in time in culture required before being delivered to a patient when compared to a population of cells produced by a density gradient centrifugation method. In some cases, the population of cells require about 1, 2, 3, 4, 5, 6, 7, or 8 fewer days in culture required before being delivered to a patient when compared to a population of cells produced by a density gradient centrifugation method.

In some cases, the exhibited increase or decrease of one or more biological properties when

compared to a population of cells produced by a density gradient centrifugation method is apparent 0, 3, 6, 9, 13, or 16 days after being produced by the methods and systems described herein.

In some cases, the methods described herein produce a population of cells that exhibit a decrease in time required to expand in culture to produce a single therapeutic dose equivalent of cells when compared to a population of cells produced by a density gradient centrifugation method.

In some cases, the methods described herein produce a population of cells that exhibit a decrease in time required to expand in culture to produce a single therapeutic dose equivalent of cells when compared to a population of cells produced by a density gradient centrifugation method.

In some cases, the methods described herein further comprises freezing and thawing the genetically engineered leukocyte population. In some cases, the methods described herein further comprise administering the genetically engineered leukocyte population to an individual afflicted with a tumor or cancer.

### EXAMPLES

The following illustrative examples are representative of embodiments of compositions and methods described herein and are not meant to be limiting in any way.

### Example 1—Deterministic Lateral Displacement Produces Cell Populations with Higher Potential for Viral Uptake and Expression

### Methods

Leukopacks (leukopheresis product) were collected the day before and stored at RT or 4° C. while rocking. When stored at 4° C., the sample was brought up to room temperature before processing.

At Day 0 the apheresis was incubated with Benzonase (Millipore-Sigma Cat #E1014) at 50 U/ml or 100 U/ml and kept rocking for 1 h until use. Aliquots were removed to measure viability with 7-AAD and total cell counts. A fraction of the Apheresis was subjected to density-based centrifugation with Ficoll (GE Cat #17-440-02) whereas the remaining of the Leukopack was processed by DLD and collected into a buffer containing 4% BSA in Plasmalyte-A. Ficoll is a neutral, highly branched, high-mass, hydrophilic polysaccharide having a density of about 1.078 g/ml.

DLD separation was performed using arrays of obstacle cartridges with hexagonal obstacles configured such that P1 for each obstacle was about 40 μm and P2 was about 20 μm. G1 was 22 μm and G2 was 17 μm. The obstacles were arranged such that the face of each hexagon was perpendicular to the axis of bulk fluid flow. Samples were flowed through the device using a peristaltic pump.

At the end of the DLD and Ficoll processing, samples were taken to determine the % of CD3+ (T cells) within the CD45+ (PBMC) fractions using the following antibody cocktail CD3-BV421, CD4-PERCP, CD45-FITC and CD8-PE.

T cells at 10×10\hat{ }6/ml from the DLD product and Ficoll product were activated by incubation with CD3/CD28 antibodies conjugated to magnetic beads (Dynabeads, Thermo-Fisher Cat #11131D) at a ratio of 3:1 beads per cell, for 1 h at 37° C. After that, the beads: cells were separated from the non-T cells using a magnet and plated at 1×10\hat{ }6/ml in TexMACS (Miltenyi Cat #130-097-196) media supplemented with 10% FBS and Pen/Strep and 50 ng/ml of both IL-7 and IL-15 (Biolegend Cat #581908, 570308) in a 6-well G-Rex plate (Wilson-Wolf Cat #P/N 80240M)

On Day 1 the plated cells were transduced with a GFP-Lentivirus at an MOI of 5 and in the presence of the TransPlus Virus Transduction Enhancer (ALSTEM Cat #V050) at 1: 500× and the cells were cultured further for another six or seven days.

At Day 3, aliquots of the cultured cells were removed and de-beaded by pipetting before counting using a Cell DYN coulter counter (Abbott). Cells were examined by flow using the following cocktail CD11b-BV570, CD69-BV510, CD3-BV421, CD4-BV650, CD45RA-BV605, CD62L-PECY7, CCR7-PE, DRAQ7, CD8-APCCY7 or by immunofluorescence microscopy by counter-staining with a CD45-A647 antibody, all from Biolegend. The remainder of the cells were fed with full media as needed. This process was repeated at Day 6 or day 7 to determine the % of T cells positive for the GFP-Lentivirus.

### Results

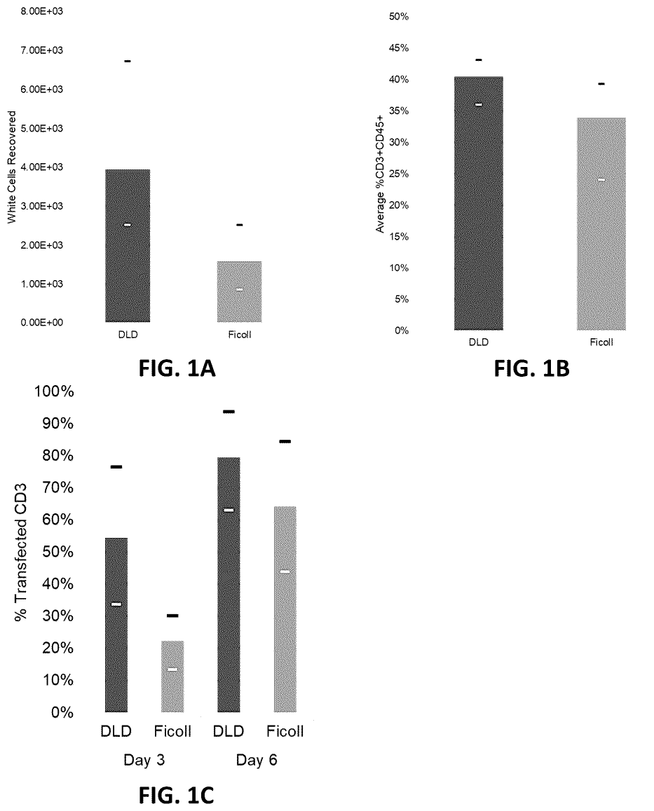

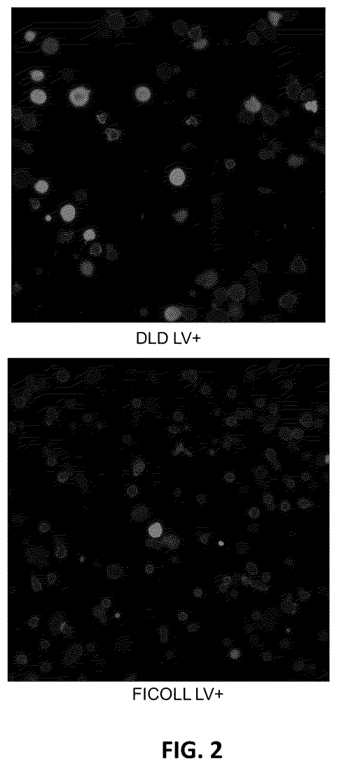

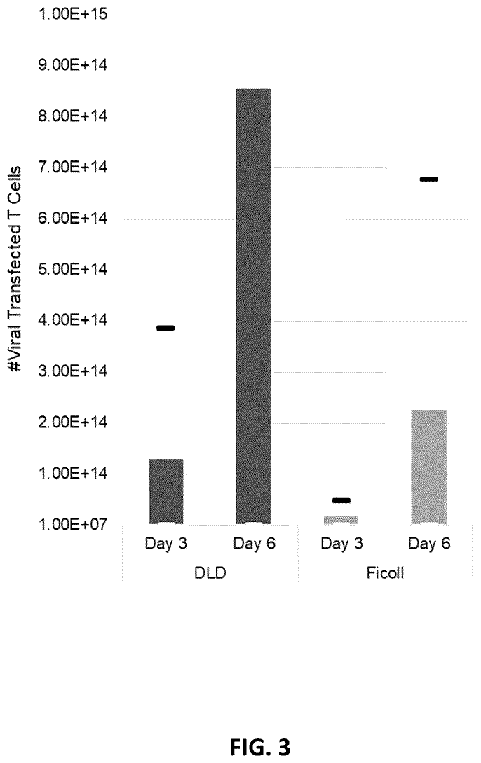

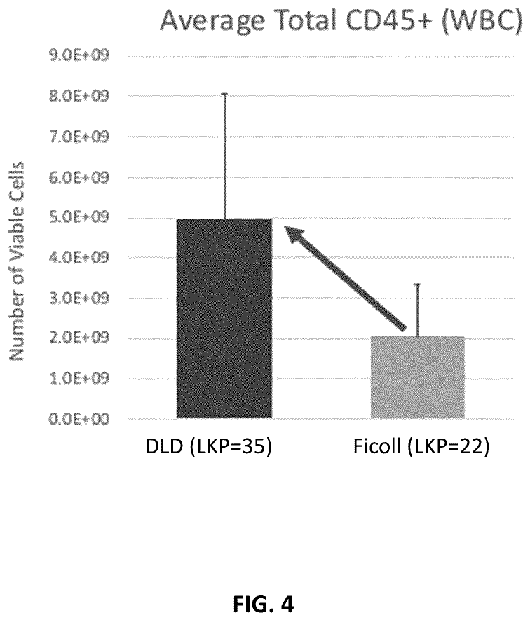

Enrichment of leukopacks by DLD resulted in greater recovery of total peripheral blood mononuclear cells as shown in FIG. 1A, and an increase (approx. 20%) in the CD3+% of total CD45+ cells recovered, as shown in FIG. 1B. After viral transduction DLD enriched target cells showed higher levels of transfection on day 3 and day 6 compared to compared to Ficoll as shown in FIG. 1C. FIG. 2 shows fluorescence microscopy of GFP positive cells at day 3 after transduction by virus for cells enriched by DLD or density gradient centrifugation. Because DLD increased the yield of total PBMCs, the percentage of CD3+ cells from the PBMCs, and the transduction efficiency compared to Ficoll, DLD resulted in a greater number of T cells transfected at both 3 days and 6 days post transfection, as shown in FIG. 3. This difference was a 10-fold increase in transfected cells at day 6.

### Example 2—Greater Numbers of CD45+ Cells CD3+ Cells are Recovered on Day 0 after DLD Isolation Compared to Density Gradient Separation

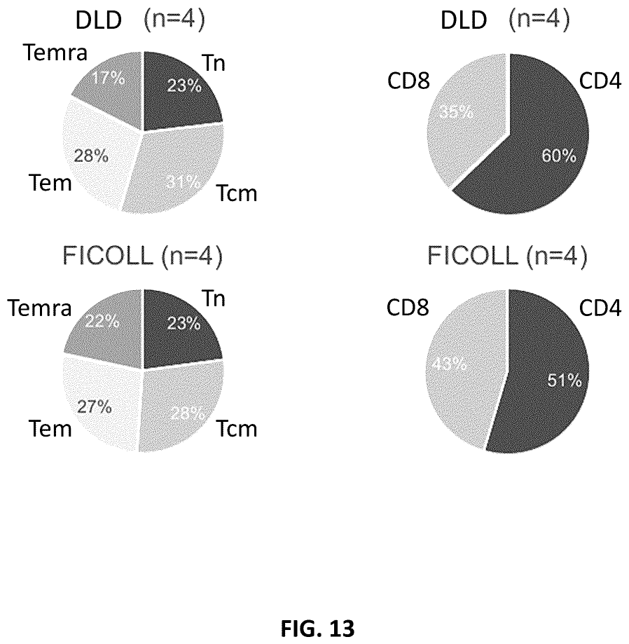

The performance of the DLD system compared to density gradient centrifugation separation (e.g., Ficoll®, GE Healthcare) with respect to total numbers of leukocytes and T cells was investigated. Compared to Ficoll, leukopaks enriched using the DLD system showed an increased amount of total viable (DARQ7−) CD 45+ cells (a pan leukocyte marker), as determined by flow cytometery. As illustrated in FIG. 13 when normalized to 1 200 mL input, an average of 5×10⁹ CD45+ cells would be isolated from leukocytes compared to 2×10⁹ for cells isolated using Ficoll, a 2.5-fold increase. As illustrated in FIG. 15, there is also an increase in viable total CD45+ and CD3+ cells (a pan-T cell marker) obtained using the DLD system compared to Ficoll when processing lower WBC count patient samples. This advantage is critical for obtaining lymphocytes from NHL, lymphoma, AML, breast cancer, colorectal cancer, or other cancer patients. Likewise, the increased lymphocyte and T cell recovery achievements of DLD can be expressed in terms of WBC ratios to cells that are desired to be depleted from input samples as a measure of debulking efficiency. As illustrated in FIG. 16, DLD products result in lower ratios of both RBC/WBC and PLT/WBC. The determination was done by flow cytometry using CD41 as a marker for platelets, CD235a for red blood cells and CD45 for white blood cells. The DLD protocol results in a white blood cell population with significantly less RBC's (red blood cells) and PLT's (platelets).

### Example—3 Greater Numbers of Beneficial T Cell Subtypes and Lowered Numbers of Undesired or Deleterious T Cell Subtypes are Recovered on Day 0 after Isolation Compared to Density Gradient Separation

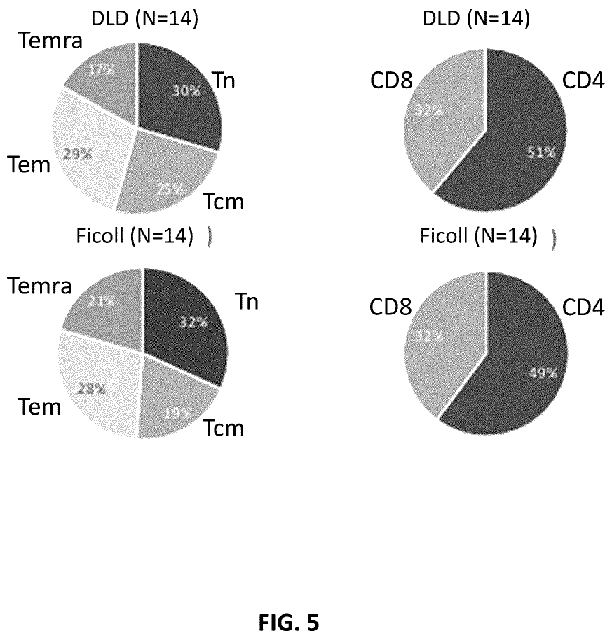

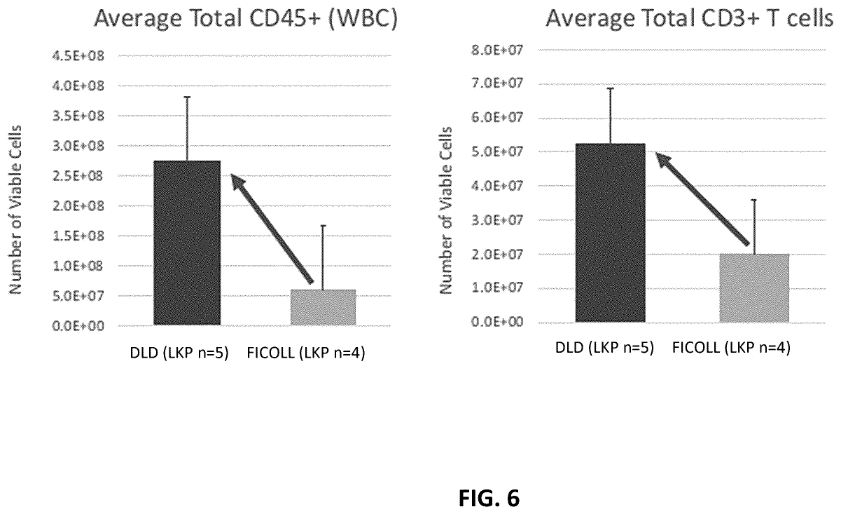

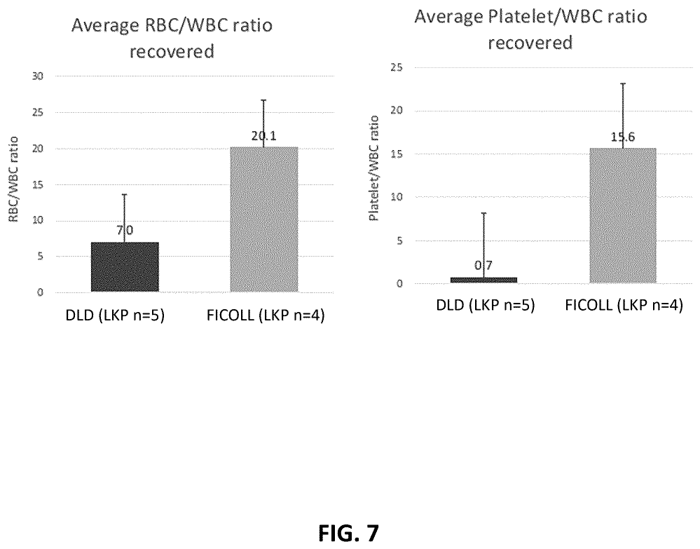

The efficacy and safety of T cell therapies depend on the T cell subtypes used in the manufacture of the therapy. Therefore, the T cell subtypes isolated using the DLD system were compared to Ficoll purification, which is a common method of isolating T cells from blood and leukapheresis samples. Compared to Ficoll, leukopaks enriched using the DLD system showed a higher percent composition of T central memory cells and less fully differentiated T effector cells. As illustrated in FIG. 5, CD4+ and CD8+ T cell populations were isolated using DLD and Ficoll methods. On average, the DLD method isolated populations were comprised of 30% T naïve cells (CD3+/CD45RA+/CCR7+), 25% T central memory cells (CD3+/CD45RA−/CCR7+), 29% T effector memory cells (CD3+/CD45RA+/CCR7−), and 17% T emra cells (effector memory differentiated) (CD3+/CD45+/CCR7−). On average, Ficoll separated populations were comprised of 32% T naïve cells (CD3+/CD45RA+/CCR7+), 19% T central memory cells (CD3+/CD45RA−/CCR7+), 28% T effector memory cells (CD3+/CD45RA+/CCR7−), and 21% T emra cells (effector memory differentiated) (CD3+/CD45+/CCR7−).

### Example 4− T Cell Populations Isolated by DLD are More Receptive to Lentiviral Transduction, are More Efficiently Transduced by Lentivirus, Express Lentiviral Delivered Genes Faster, and Retain

### More Beneficial T Cell Subtypes as Compared to Density Gradient Separation

The timely administration, efficacy, and safety of T cell therapies depend on how amenable isolated T cells are to genetic engineering and subsequent expansion, along with how quickly and efficiently they can express heterologous genetic material. Therefore, the T cell responses to lentiviral transduction, using T cells isolated using the DLD system, were compared to those obtained from Ficoll purification. Leukopacks from three different donors were processed, had T cells isolated/activated with CD3/CD28 beads, and were transduced with GFP-Lentivirus. Cell were then expanded in cell culture with IL-7/IL-15 over the course of 9 days. As illustrated in FIG. 8, Cells were analyzed by flow cytometry at the corresponding days, to monitor the uptake and integration of the GFP-Lentivirus and compared to non-transduced cells at 0, 3, 6, 9, and 12 days, showing that DLD-prepared cell populations more readily integrate lentivirus, including at Day 6 which showed a 30% increase in number of integrated cells as compared to Ficoll-prepared cells. FIG. 10 shows the average % of transduced cells in the DLD and Ficoll prepared cell populations, showing that DLD populations are more amenable to lentiviral transduction, in some cases, by 20-100%.

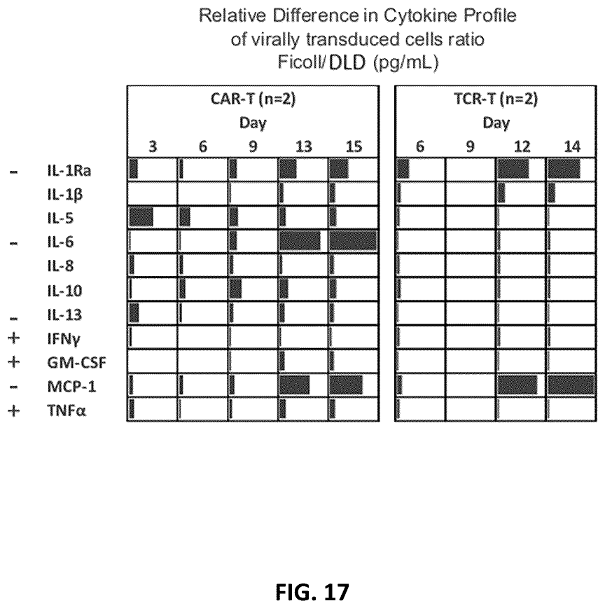

These findings were confirmed using a 9-day time course of immunofluorescence microscopy. T cells from DLD and Ficoll procedures, from the same donor, were isolated/activated and transduced with GFP-Lentivirus and expanded in cell culture. At the indicated times, the cells were examined by microscopy to monitor the GFP-Lentivirus uptake and expression of GFP, showing that DLD cells are more easily transduced than Ficoll cells, as indicated by greater GFP signal in DLD-derived cell populations illustrated in FIG. 9. Thus, cells prepared by the DLD system are able to be more easily transduced as compared to other systems methods. (see FIGS. 17-19). DLD produced cells are consistently more easily transduced, about 87.5% showing significant improvement versus Ficoll. The average improvement at day 3 was “2 fold, and at day 6, 9 a 30% advantage was retained. At all times the DLD system produced cells averaged a higher transduction level.

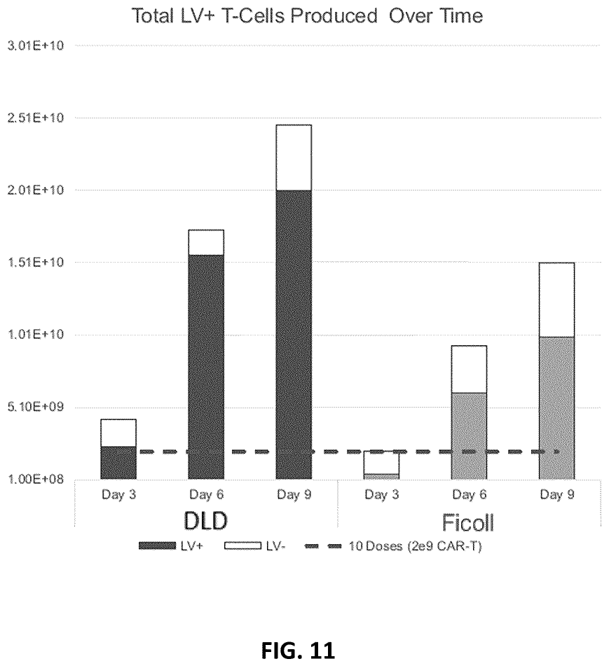

These findings are especially salient in translating these separation processes to clinical applications. Greater lentiviral transduction efficiency leads to reduced times to dosing i.e. obtaining sufficient numbers of cells for a dose or multiple doses of therapeutic cells. As illustrated in FIG. 11, DLD methods can produce enough lentiviral transduced cells equivalent to 10 doses of therapeutic cells after 3 days in culture and more total doses than Ficoll over a period of 9 days, normalized to an initial input of 200 mL of leukopack material. In some cases, the DLD-prepared cell populations yielded twice as many transduced cells over a period of 9 days.

In addition to the number of total cells produced after separation and lentiviral transduction, it is crucial that therapeutic cell doses comprise effectively activated T cell types, such as T central memory and T effector memory cells. The T cell subsets composition of GFP-Lv+ cells at day 3 and day 6 after activation were compared between DLD and Ficoll-prepared cell populations. Determination of T cell subtypes was done by flow cytometry within the GFP-Lv+ T cells, as illustrated in FIG. 12. At Day 6, DLD methods result in a population of T cells comprising 4% T naïve (CD3+/CD45RA+/CCR7+), 19% T central memory (CD3+/CD45RA−/CCR7+), 74% T effector memory (CD3+/CD45RA+/CCR7−), and 3% T emra (CD3+/CD45+/CCR7−). At Day 6, Ficoll methods result in a population of T cells comprising 28% T naïve (CD3+/CD45RA+/CCR7+), 16% T central memory (CD3+/CD45RA−/CCR7+), 51% T effector memory (CD3+/CD45RA+/CCR7−), and 5% T emra (CD3+/CD45+/CCR7−). Thus, DLD cells have a larger pool of T cm resulting in a more robust conversion to T em cells as compared to Ficoll GFP=Lv+ T cells.

These findings were confirmed with additional experiments comparing cell population T Cell compositions from DLD and Ficoll-prepared compositions (from healthy donors) prior to lentiviral transduction (FIG. 13) and at 3, 6, and 9 days post transduction in culture (FIG. 14). Day 0 (pre-transduction) DLD cells showed a higher number of CD4+ cells and less differentiated T cm cells than Ficoll cells as determined by flow cytometry. Progression of the T cell subtypes with GFP-Lv is illustrated in FIG. 14. Viable CD3+ cells from DLD protocol showed a bias towards Tcm, over time as compared to the cells from the Ficoll protocol. GFP-Lv+ and T cell subsets were determined by flow cytometry. For example, on Day 9, DLD methods result in a population of T cells comprising 5% T naïve (CD3+/CD45RA+/CCR7+), 29% T central memory (CD3+/CD45RA−/CCR7+), 59% T effector memory (CD3+/CD45RA+/CCR7−), and 7% T emra (CD3+/CD45+/CCR7−). At Day 9, Ficoll methods result in a population of T cells comprising 9% T naïve (CD3+/CD45RA+/CCR7+), 20% T central memory (CD3+/CD45RA−/CCR7+), 59% T effector memory (CD3+/CD45RA+/CCR7−), and 12% Temra (CD3+/CD45+/CCR7−).

### Example 5− T Cell Populations Isolated by DLD have Lower Expression of Cell Senescence and Exhaustion Markers after Activation, Lentiviral Transduction, and Expansion as Compared to T Cell Populations Prepared with Ficoll Methods

Efficacy and manufacture of therapeutic cells relies partially on having populations of viable and expanding cells i.e. not senescent or exhausted T cells. Activated, transduced and expanded T cells were examined for senescence (CD57+/KLRG1+) and exhaustion (CD57/KLRG1+/PD1+/Tim3+) at day 13 and expressed as a ratio of Ficoll/DLD. Ficoll cells transduced with the full CAR19 signaling domain had a more pronounced expression senescence and exhaustion markers (CD57+/KLRG1+ and CD57−/KLRG1+ with PD1 and Tim3 co-expression) than DLD cells, as illustrated in FIG. 15, whereas there was very little difference in cells transduced with the controls of NO CAR or inactive CAR (CAR19-Sig domain).

### Example 5− T Cell Populations Isolated by DLD have Comparable or Increased Killing Capacity as Compared to Those Prepared Using Ficoll Methods

Efficacy of therapeutic T cell preparations relies on those cells being effective at killing cells comprising a targeted peptide sequence. T cells from DLD or Ficoll were isolated, activated and transduced with a TCRT lentivirus specific for the MART-1 antigen. At day 6 cells were collected and co-cultured with T2 target cells (Luc+) bearing the MART-1 peptide at different ratios. Upon incubation the T2 cell mortality was examined by the loss of chemiluminescence in the co-culture. Both DLD and Ficoll cells were capable of killing their target cells in a dose-dependent manner. As illustrated in FIG. 16, cells prepared using DLD methods exhibited higher killing capacities at 2:1, 1:1, and 0.5:1 T cells to target cell ratios than those prepared using Ficoll, a 30% increase in killing in some cases.

### Example 6− T Cell Populations Isolated by DLD have Higher Desirable Cytokine Expression and Lower Undesirable Cytokine Expression as Compared to T Cell Populations Isolated by Ficoll

Therapeutic T cell preparation safety relies partly on isolating cells that will result in more cell killing activity as opposed to inflammatory responses in order to avoid adverse effects to patients. As such, it is desirable that T cell populations prepared using various isolation methods express more cell-killing cytokines and have low inflammatory response cytokine expression. As such, cytokine expression was compared between T cell populations isolated using DLD and Ficoll methods.

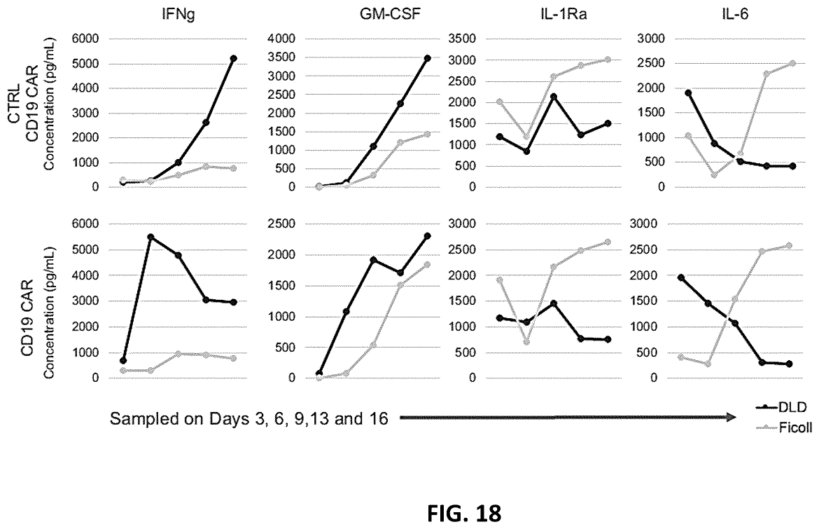

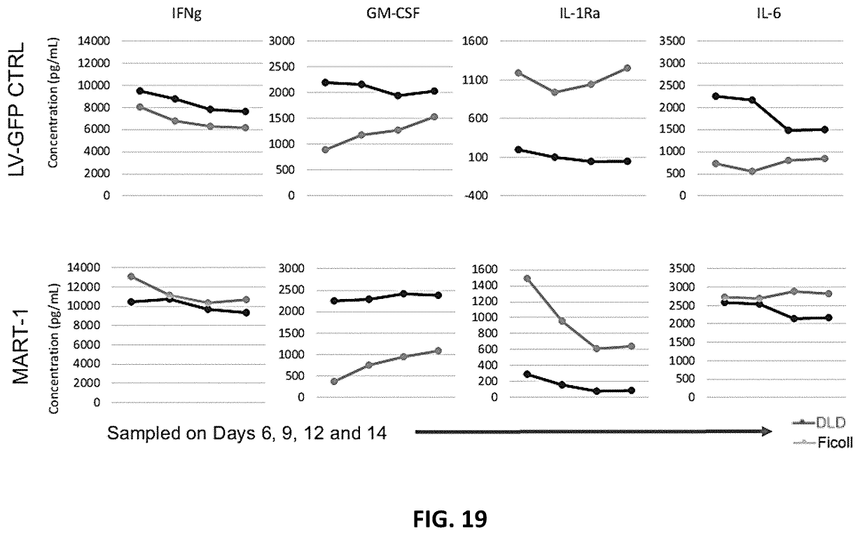

Supernatants from DLD and Ficoll cells were collected at days 0, 6 and 13 after T cell isolation/activation, expansion (IL7/IL-15), and lentiviral transduction (CAR-T-CD19 or TCRT-MART-1). 15 different cytokines were analyzed by a Luminex multiplex assay for all of the supernatants. The results are expressed as the ratio of Ficoll/DLD (pg/ml), as illustrated in FIG. 17. Time course cytokine expression for IFNg, GM-CSF, IL-1Ra, and IL-6 in CAR-T-CD19 transduced cells is illustrated in FIG. 18. Time course cytokine expression for IFNg, GM-CSF, IL-1Ra, and IL-6 in TCRT-MART-1 transduced cells is illustrated in FIG. 19. Ficoll cells secreted more IL-6, MCP-1 and IL-1Ra involved in the inflammatory response, whereas DLD cells expressed more IFNg and GM-CSF, typical markers of cell killing activity. Thus, DLD-prepared T cell populations exhibit a more favorable cytokine expression profile.

### Example 7—T Cell Populations Isolated by the Methods Described Herein have Longer Telomeres

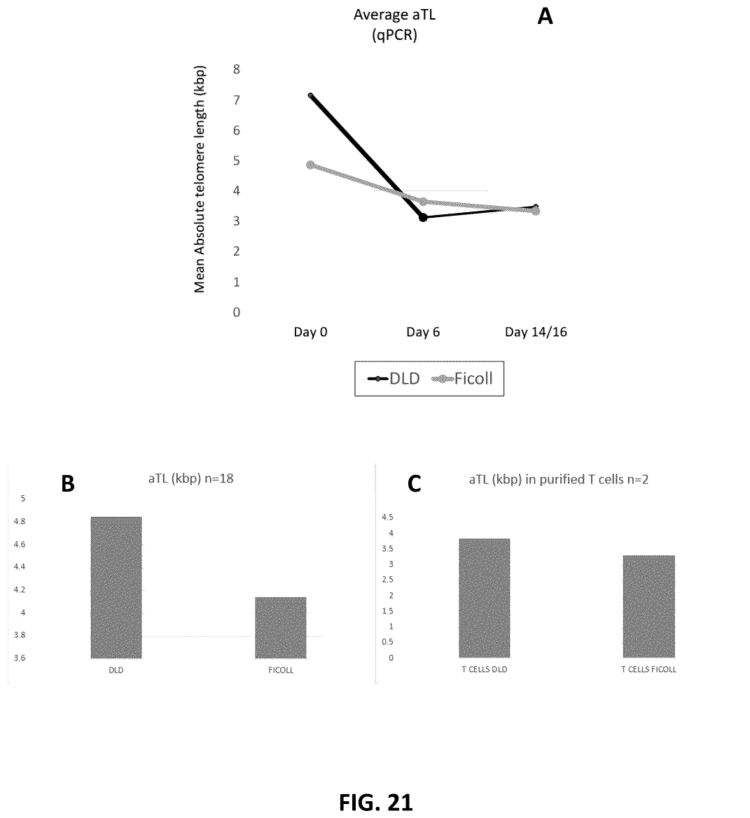

As shown in FIG. 21A cells isolated by the methods described herein possess longer telomere length compared to Ficoll, indicating greater expansion capability. Assay was conducted using qPCR analysis for absolute Telomere Length (aTL).

A subsequence analysis confirmed that DLD cells have a longer aTL than Ficoll cells. FIG. 21B T cells also have a longer aTL when purified from the DLD enriched population rather than the Ficol enriched population. FIG. 21C.

### Example 8—T Storage and Recovery of Cell Populations Produced by DLD Separation

Cells separated by DLD were counted and centrifuged at 400×g for 5 min. Cells were resuspended in cold CS-10 and diluted to a final concentration of 150×10⁶ cells/mL. Cryovials were filled with 1 mL of suspension and gradually chilled to −80° C. Cryovials were then stored in the vapor phase of liquid nitrogen. Alternatively, cells at 50×10⁶/ml can be frozen in 90% FBS+10% DMSO.

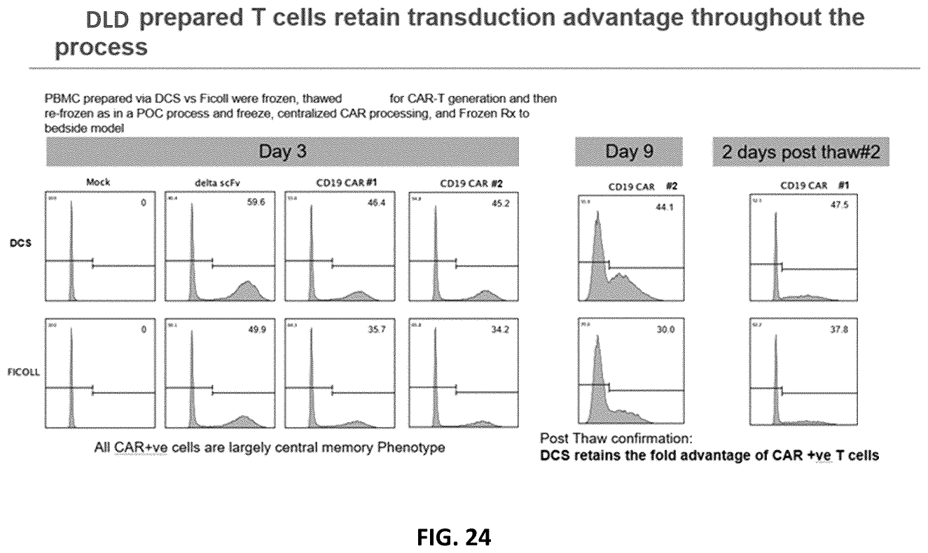

Frozen cells were shipped to a remote laboratory and thawed in media. T-cells were selected and activated using CD3/CD28 dual purpose beads. Alternatively, T cells can be selected with non-T cell receptor targets ((i.e. CD4, CD8) and subsequently activated with CD3/CD28. The activated T cells were transduced using a polybrene transduction enhancer with a recombinant virus for CAR expression. The CAR T cells were then frozen, shipped back to the main lab, and analyzed by flow cytometry, which confirmed that the twice frozen cells were functional killers, as shown in FIG. 24.

### Example 9—CAR-T Therapeutics Produced Using DLD Separations

A fresh sample is separated by DLD. The resulting cells are frozen and shipped to the site where the therapeutic will be produced. At the site, the cells are thawed and recovered in media. The cells are activated and selected to add virus along with an optional transduction enhancer. The virus is integrated and the cells are again frozen. The cells are then transported to the therapeutic site, where they are thawed, and expression of CAR is confirmed prior to use.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A method for obtaining a genetically engineered leukocyte composition comprising:
(a) enriching a population of large cells from a biological sample obtained from a subject, wherein the biological sample comprises leukocytes without performing density gradient centrifugation;
(b) contacting the population of large cells with an activating agent; and
(c) transducing the population of large cells with a viral vector comprising a polynucleotide, wherein
the enriching comprises an array-based separation comprising a microfluidic device configured for deterministic lateral displacement comprising an array of obstacles, and
the obstacles are elongated so that their length perpendicular to bulk fluid flow (P1) is longer than their width parallel to bulk fluid flow (P2) by at least 10%.

2. The method of claim 1, wherein the enriching comprises removing components below a predetermined size from the biological sample.

3. The method of claim 1, wherein the biological sample is:
(a) a leukopak;
(b) residual leukocytes from a platelet donation;
(c) a blood sample; or
(d) an apheresis sample.

4. The method of claim 1, wherein the polynucleotide:
(a) is a heterologous DNA or a heterologous RNA;
(b) encodes a CRISPR guide RNA;
(c) encodes a polypeptide; or
(d) encodes a chimeric antigen receptor.

5. The method of claim 1, wherein at least 90% of the cells of the genetically engineered leukocyte composition are viable.

6. The method of claim 1, wherein the microfluidic device comprises a plurality of arrays
comprising a plurality of obstacles arranged into rows running approximately perpendicular to a direction of fluid flow and columns running approximately parallel to the direction of fluid flow, wherein the columns are offset from the direction of fluid flow by a tilt angle.

7. The method of claim 1, further comprising culturing the population of large cells.

8. The method of claim 1, wherein the large cells are T-cells, the polynucleotide encodes a polypeptide, and at least 70% of the T-cells express the polypeptide.

9. The method of claim 1, wherein the large cells are T-cells and at least 70% of the T-cells express the polynucleotide.

10. The method of claim 1, wherein the viral vector is a lentiviral vector, an adenovirus vector, or an adeno-associated viral (AAV) vector.

11. The method of claim 1, wherein the subject is a human.

12. A cell population obtainable by the method of claim 1, wherein the cell population comprises the polynucleotide, wherein compared to a buffy coat cell population from the biological sample by density gradient centrifugation:
a. a percentage of T effector memory cells that express CD45Ra in the cell population is at least 10% less than a percentage of T effector memory cells that express CD45Ra in the buffy coat cell population;
b. a percentage of cells comprising the polynucleotide in the cell population is at least 20% higher than a percentage of cells comprising the polynucleotide in the buffy coat cell population; and the cell population and the buffy coat cell population are transduced with the viral vector comprising the polynucleotide; or
c. the cell population comprises T cells comprising a higher mean absolute telomer length than T cells purified from the buffy coat cell population.

13. The cell population obtainable by the method of claim 10, wherein:
a. a percentage of cells comprising the polynucleotide in the cell population is at least 20% higher than a percentage of cells comprising the polynucleotide in the buffy coat cell population; and
b. the cell population and the buffy coat cell population are transduced with a viral vector comprising the polynucleotide.

14. The cell population obtainable by the method of claim 10, wherein the density gradient centrifugation comprises layering the biological sample over an aqueous solution comprising sodium diatrizoate, disodium calcium EDTA, and a neutral, highly branched, high-mass, hydrophilic polysaccharide having a density of about 1.078 grams per milliliter (g/mL).
